(19) United States
(12) Patent Application Publication
(10) Pub. No.: US 2024/0183860 A1
(43) Pub. Date: Jun. 6, 2024

(54) Chimeric ABC Transporters and Screening Methods

(71) Applicant: Genentech, Inc., South San Francisco, CA (US)

(72) Inventors: Jian Mehr-Dean Payandeh, Belmont, CA (US); Patrick C. Reid, Tokyo (JP); Christian Nathaniel Cunningham, Oakland, CA (US); Evan Green, South San Francisco, CA (US); Christopher Koth, Pacifica, CA (US); Vishal Verma, San Carlos, CA (US)

(73) Assignee: Genentech, Inc., South San Francisco, CA (US)

(21) Appl. No.: 18/493,957

(22) Filed: Oct. 25, 2023

Related U.S. Application Data
(63) Continuation of application No. PCT/US2022/026319, filed on Apr. 26, 2022.
(60) Provisional application No. 63/315,255, filed on Mar. 1, 2022.
(60) Provisional application No. 63/213,652, filed on Jun. 22, 2021.
(60) Provisional application No. 63/181,118, filed on Apr. 28, 2021.

(51) Int. Cl. G01N 33/68; A61K 38/00; C07K 7/56

(52) U.S. Cl. CPC G01N 33/6845; C07K 7/56; G01N 33/6872; A61K 38/00

(57) ABSTRACT

The present disclosure relates to chimeric ABC transporter proteins and methods of screening for molecules that bind to the periplasmic, extracellular, and/or luminal face of an ABC transporter protein using the chimeric ABC transporters. For example, in some embodiments, screening methods involve providing a chimeric ABC transporter in which one or more regions of the periplasmic, extracellular, and/or luminal face of the ABC transporter are substituted with one or more equivalent regions of the periplasmic, extracellular, and/or luminal face of a different ABC transporter and selecting for molecules that bind to the ABC transporter but do not bind to the chimeric ABC transporter. The disclosure also relates to molecules that bind to the periplasmic, extracellular, and/or luminal face of an ABC transporter protein, for example, identified in such screens.

### CROSS-REFERENCE TO RELATED APPLICATIONS

This application is a continuation of International Patent Application No. PCT/US2022/026319, filed Apr. 26, 2022, which claims the benefit of priority of U.S. Provisional Application No. 63/181,118, filed Apr. 28, 2021, U.S. Provisional Application No. 63/213,652, filed Jun. 22, 2021, and U.S. Provisional Application No. 63/315,255, filed Mar. 1, 2022, the contents of each of which are incorporated by reference herein in their entireties for any purpose.

### SEQUENCE LISTING

The instant application contains a Sequence Listing which has been submitted electronically in XML format and is hereby incorporated by reference in its entirety. Said XML copy, created on Oct. 23, 2023, is named “2023-10-23_01164-0015-00US_SeqList.xml” and is 42,148 bytes in size.

### FIELD

The present disclosure relates to chimeric ABC transporter proteins and methods of screening for molecules that bind to the periplasmic, extracellular, and/or luminal face of an ABC transporter protein using the chimeric ABC transporters. For example, in some embodiments, screening methods involve providing a chimeric ABC transporter in which one or more regions of the periplasmic, extracellular, and/or luminal face of the ABC transporter are substituted with one or more equivalent regions of the periplasmic, extracellular, and/or luminal face of a different ABC transporter and selecting for molecules that bind to the ABC transporter but do not bind to the chimeric ABC transporter. The disclosure also relates to molecules that bind to the periplasmic, extracellular, and/or luminal face of an ABC transporter protein, for example, identified in such screens.

### BACKGROUND

ATP binding cassette (“ABC”) transporters constitute a superfamily of integral membrane proteins found in prokaryotes and eukaryotes and in eukaryotic organelles that are responsible for the ATP-powered translocation of many types of substrates across membranes. ABC transporters have a transmembrane pore that is either accessible from the inner face of a membrane (inward-facing conformation) or from the outer face of a membrane (outward-facing conformation). ATP hydrolysis drives conformational changes in the protein, resulting in “flipping” the transmembrane pore from the inner side to the outer side of the membrane or vice versa for transport of substrates across the membrane. See, e.g., FIG. 6.

Gram-positive bacterial cells have a single cytoplasmic membrane. Gram-negative bacterial cells have an outer membrane and an inner/cytoplasmic membrane. The space between the outer and inner membranes is the periplasm. In Gram-negative bacterial cells, for example, ABC transporters may be used to flip particular substrates from the outer membrane to the inner membrane and vice versa, such as the transport of lipopolysaccharides (LPS) between the outer and inner (cytoplasmic) membranes. Eukaryotic cells, in contrast, have a single membrane separating the exterior of the cell from the cytosol. Eukaryotic organelles also generally have a single membrane that separates the cytosol from the organellar lumen.

It may be desirable to inhibit the activity of ABC transporters for various reasons. For example, a molecule that could inhibit certain ABC transporters in bacteria could be an effective antibiotic. One possible antibiotic target is MsbA, a highly conserved ABC transporter in Enterobacteriaceae (Gram-negative) that transports lipopolysaccharides (LPS) from the cytoplasm across the inner membrane/cytoplasmic membrane to the periplasm for incorporation into the outer membrane. MsbA is a major component of the outer membrane and essential for Gram-negative bacterial growth; depletion of MsbA disrupts LPS trafficking and causes cell lysis, and blocking the MsbA ATPase activity inhibits bacterial growth. MsbA could be a target for antibiotics because it has low identity with human ABC transporters; for example, human P-gp is only about 30% identical to MsbA.

Previous high-throughput screening efforts in Gram-negative bacteria to identify molecules that bind to and inhibit the activity of ABC transporters such as MsbA utilized mutant cell types that had more permeable outer membranes, for example, the imp Escherichia coli strain. FIG. 3E. These screens tended to be biased toward identifying molecules that bind the inward-facing conformation of a bacterial ABC transporter. This presented a problem because in order to bind to the inward-facing conformation in a Gram-negative cell, the molecule would first need to cross both the outer membrane and the inner/cytoplasmic membrane, and most molecules identified were unable to do so and were thus ineffective in inhibiting the activity of ABC transporters in wild-type cells without uncharacteristically permeable outer membranes. FIG. 6. Similarly, a molecule that binds the inward-facing conformation in a Gram-positive bacterial cell or a eukaryotic cell must cross the cell membrane before binding its target. There is a need for a drug discovery method that can accommodate high-throughput screening techniques that select molecules that bind a solvent-accessible region on ABC transporters that is exposed when in the outward-facing conformation: the periplasmic, extracellular and/or luminal face, including the periplasmic, extracellular and/or luminal cleft found within the periplasmic, extracellular, and/or luminal face of the protein. Molecules that bind to this portion of an ABC transporter need not cross as many membranes to bind their targets, yet may also in some cases act as inhibitors of the ABC transporter, for instance, by competing with the transporter's normal substrate for binding or by blocking access to the normal substrate binding site on the outer face of the protein.

### SUMMARY

To screen for molecules that bind to the periplasmic, extracellular, and/or luminal face of an ABC transporter, the inventors developed a screening strategy using chimeric ABC transporter proteins in which one or more regions of the periplasmic, extracellular, and/or luminal face of the ABC transporter are substituted with one or more equivalent regions of the periplasmic, extracellular, and/or luminal face of a different ABC transporter. Such molecules may be used, for example, in a counter selection screen in which molecules that bind to the ABC transporter but that do not bind to the chimeric ABC transporter under the screening conditions are identified as molecules that bind to the periplasmic, extracellular, and/or luminal face of the ABC transporter. Described herein are chimeric ABC transporters and methods of using them to identify test molecules that bind the periplasmic, extracellular, and/or luminal face regions of an ABC transporter and associated binding molecules, molecular complexes, kits, and methods of using the identified molecules.

The present disclosure includes, for example, any one or a combination of the following embodiments:
Embodiment 1. A method of determining whether a test molecule binds to the periplasmic, extracellular, and/or luminal face of a parental ABC transporter, comprising: a) providing a chimeric ABC transporter, in which one or more regions of the periplasmic, extracellular, and/or luminal face of the parental ABC transporter are substituted with one or more equivalent regions of the periplasmic, extracellular, and/or luminal face of a different ABC transporter; and b) contacting the chimeric ABC transporter with a test molecule that binds to the parental ABC transporter in an outward-facing conformation, wherein the test molecule is determined to bind to the periplasmic, extracellular, and/or luminal face of the parental ABC transporter if the test molecule does not bind to the chimeric ABC transporter.
Embodiment 2. A method of determining whether a test molecule binds to the periplasmic, extracellular, and/or luminal face of a parental ABC transporter, comprising: a) trapping the parental ABC transporter in an outward-facing conformation; b) selecting a test molecule that binds to the parental ABC transporter in the outward-facing conformation; c) providing a chimeric ABC transporter, in which one or more regions of the periplasmic, extracellular, and/or luminal face of the parental ABC transporter are substituted with one or more equivalent regions of the periplasmic, extracellular, and/or luminal face of a different ABC transporter; and d) contacting the chimeric ABC transporter with the test molecule of (b) that binds to the parental ABC transporter in an outward-facing conformation, wherein the test molecule is determined to bind to the periplasmic, extracellular, and/or luminal face of the parental ABC transporter if the test molecule does not bind to the chimeric ABC transporter.
Embodiment 3. The method of embodiments 1 or 2, further comprising trapping the chimeric ABC transporter in an outward-facing conformation prior to contacting the chimeric ABC transporter with the test molecule.
Embodiment 4. The method of any one of embodiments 2-3, wherein the parental ABC transporter and/or the chimeric ABC transporter is trapped in an outward-facing conformation by treating the parental ABC transporter and/or the chimeric ABC transporter with Mg²⁺, ATP, and vanadate.
Embodiment 5. The method of any one of embodiments 1-4, wherein the parental ABC transporter is a Type IV or Type V ABC transporter.
Embodiment 6. The method of any one of embodiments 1-5, wherein the parental ABC transporter is a Type IV ABC transporter.
Embodiment 7. The method of any one of embodiments 1-6, wherein the parental ABC transporter is from a Gram-negative bacteria.
Embodiment 8. The method of embodiment 7, wherein the Gram-negative bacteria is selected from Escherichia coli, Enterobacter cloacae, Klebsiella pneumoniae, Pseudomonas aeruginosa, Pseudomonas psychrotolerans, Candidatus accumulibacter, Janthinobacterium agaricidamnosum, Thiomicrospira cyclica, or Magnetospira sp. strain-QH-2.
Embodiment 9. The method of embodiment 8, wherein the parental ABC transporter is from Escherichia coli.
Embodiment 10. The method of any one of embodiments 7-9, wherein the parental ABC transporter is MsbA.
Embodiment 11. The method of any one of embodiments 7-10, wherein the different ABC transporter is from a Gram-negative bacteria.
Embodiment 12. The method of embodiment 11, wherein the Gram-negative bacteria is selected from Escherichia coli, Enterobacter cloacae, Klebsiella pneumoniae, Pseudomonas aeruginosa, Pseudomonas psychrotolerans, Candidatus accumulibacter, Janthinobacterium agaricidamnosum, Thiomicrospira cyclica, or Magnetospira sp. strain-QH-2.
Embodiment 13. The method of any one of embodiments 1-12, wherein the chimeric ABC transporter differs from the parental ABC transporter in that at least one periplasmic, extracellular, or luminal loop and up to 50% of the transmembrane segments on either side of the loop are replaced with equivalent regions of the different ABC transporter.
Embodiment 14. The method of embodiment 13, wherein the chimeric ABC transporter differs from the parental ABC transporter in that at least one periplasmic, extracellular, or luminal facing loop and 50% of the transmembrane segments on either side of the loop are replaced with equivalent regions of the different ABC transporter.
Embodiment 15. The method of embodiment 13, wherein the chimeric ABC transporter differs from the parental ABC transporter in that at least one periplasmic, extracellular, or luminal facing loop and up to 25% of the transmembrane segments on either side of the loop are replaced with equivalent regions of the different ABC transporter.
Embodiment 16. The method of embodiment 13, wherein the chimeric ABC transporter differs from the parental ABC transporter in that at least one periplasmic, extracellular, or luminal facing loop and up to 10% of the transmembrane segments on either side of the loop are replaced with equivalent regions of the different ABC transporter.
Embodiment 17. The method of embodiment 13, wherein the chimeric ABC transporter differs from the parental ABC transporter in that at least two, at least three, or all of the periplasmic, extracellular, or luminal facing loops and up to 50% of the transmembrane segments on either side of each loop are replaced with equivalent regions of the different ABC transporter.
Embodiment 18. The method of any one of embodiments 1-17, wherein the parental ABC transporter and the different ABC transporter are respectively encoded by homologous genes from two different species.
Embodiment 19. The method of any one of embodiments 1-12, wherein the parental ABC transporter is E. coli MsbA (EcMsbA) and the chimeric ABC transporter is EcMsbA in which one or more of EcMsbA residues Leu47-Pro68 in periplasmic loop 1 (L1), EcMsbA residues Met159-Leu171 in periplasmic loop 3 (L3), and EcMsbA residues Ala262-Ile292 in periplasmic loop 5 (L5) are replaced with equivalent regions of Pseudomonas psychrotolerans (PpMsbA).
Embodiment 20. The method of any one of embodiments 1-12, wherein the parental ABC transporter is EcMsbA and the chimeric ABC transporter is EcMsbA in which one or more of EcMsbA residues Leu47-Pro68 in periplasmic loop 1 (L1), EcMsbA residues Met159-Leu171 in periplasmic loop 3 (L3), and EcMsbA residues Ala262-Ile292 in periplasmic loop 5 (L5) are replaced with equivalent regions of Candidatus accumulibacter (CaMsbA).
Embodiment 21. The method of any one of embodiments 1-12, wherein the parental ABC transporter is EcMsbA and the chimeric ABC transporter is EcMsbA in which one or more of EcMsbA residues Leu47-Pro68 in periplasmic loop 1 (L1), EcMsbA residues Met159-Leu171 in periplasmic loop 3 (L3), and EcMsbA residues Ala262-Ile292 in periplasmic loop 5 (L5) are replaced with equivalent regions of Janthinobacterium agaricidamnosum (JaMsbA).
Embodiment 22. The method of any one of embodiments 1-12, wherein the parental ABC transporter is EcMsbA and the chimeric ABC transporter is EcMsbA in which one or more of EcMsbA residues Leu47-Pro68 in periplasmic loop 1 (L1), EcMsbA residues Met159-Leu171 in periplasmic loop 3 (L3), and EcMsbA residues Ala262-Ile292 in periplasmic loop 5 (L5) are replaced with equivalent regions of Thiomicrospira cyclica (TcMsbA).
Embodiment 23. The method of any one of embodiments 1-12, wherein the parental ABC transporter is EcMsbA and the chimeric ABC transporter is EcMsbA in which one or more of EcMsbA residues Leu47-Pro68 in periplasmic loop 1 (L1), EcMsbA residues Met159-Leu171 in periplasmic loop 3 (L3), and EcMsbA residues Ala262-Ile292 in periplasmic loop 5 (L5) are replaced with equivalent regions of Magnetospira strain-QH-2 (MqMsbA).
Embodiment 24. The method of any one of embodiments 1-23, wherein the molecule binds to the periplasmic, extracellular, or luminal cleft of the parental ABC transporter.
Embodiment 25. The method of any one of embodiments 1-24, wherein the method further comprises conducting an ATPase assay of the parental ABC transporter in the presence of the molecule.
Embodiment 26. The method of any one of embodiments 1-25, wherein the method further comprises conducting a cell viability or growth assay and/or an ABC transporter functional assay of the parental ABC transporter in the presence of the molecule.
Embodiment 27. A molecule identified by the method of any one of embodiments 1-26, wherein the molecule binds to the parental ABC transporter with a K_{D} of 20 μM or less.
Embodiment 28. A molecule of embodiment 27, wherein the molecule binds to the parental ABC transporter with a K_{D} of 10 μM or less.
Embodiment 29. A molecule of embodiment 27, wherein the molecule binds to the parental ABC transporter with a K_{D} of 20 nM or less.
Embodiment 30. A molecule of embodiment 27, wherein the molecule binds to the parental ABC transporter with a K_{D} of 500 nM or less.
Embodiment 31. A molecule of embodiment 27, wherein the molecule binds to the parental ABC transporter with a K_{D} of 1 nM or less.
Embodiment 32. A molecule of embodiment 27, wherein the molecule binds to the parental ABC transporter with a K_{D} of 1 to 20 μM.
Embodiment 33. A molecule of embodiment 27, wherein the molecule binds to the parental ABC transporter with a K_{D} of 10 to 20 μM.
Embodiment 34. A molecule of embodiment 27, wherein the molecule binds to the parental ABC transporter with a K_{D} of 1 nM to 20 μM.
Embodiment 35. A molecule of embodiment 27, wherein the molecule binds to the parental ABC transporter with a K_{D} of 1 nM to 500 nM.
Embodiment 36. A molecule identified by the method of any one of embodiments 1-26, wherein the molecule is a peptide.
Embodiment 37. A molecule identified by the method of any one of embodiments 1-26, wherein the molecule is a small molecule.
Embodiment 38. A molecule identified by the method of any one of embodiments 1-26, wherein the molecule is an antibody.
Embodiment 39. A molecule identified by the method of any one of embodiments 1-26, wherein the molecule is a binding fragment of a peptide, small molecule, or antibody.
Embodiment 40. The peptide of embodiment 36, wherein the peptide is a macrocycle.
Embodiment 41. The macrocycle of embodiment 40, wherein the macrocycle is a 6-14-mer, 6-10-mer, 6-8-mer, or 8-10-mer macrocycle.
Embodiment 42. The macrocycle of embodiment 40 or 41, wherein the macrocycle has at least one lipophilic side-chain and at least one positively charged side-chain.
Embodiment 43. The molecule of any one of embodiments 27-42, wherein the molecule binds to the periplasmic, extracellular, or luminal cleft of the parental ABC transporter.
Embodiment 44. A macrocycle peptide G1118, G1119, or G1122.
Embodiment 45. A macrocycle peptide G1365.
Embodiment 46. A molecule that competes with macrocycle G1118, G1119, and/or G1122 for binding to the periplasmic face of a parental ABC transporter, when the parental ABC transporter is trapped in an outward-facing conformation by treatment with Mg²⁺, ATP, and vanadate.
Embodiment 47. The molecule of embodiment 46, wherein the molecule inhibits binding of G1118, G1119, and/or G1122 to the parental ABC transporter by at least 50, 60, 70, 80, 90 or 100% in a competition assay.
Embodiment 48. A molecule that competes with macrocycle G1365 for binding to the periplasmic face of a parental ABC transporter, when the parental ABC transporter is trapped in an outward-facing conformation by treatment with Mg²⁺, ATP, and vanadate.
Embodiment 49. The molecule of embodiment 48, wherein the molecule inhibits binding of G1365 to the parental ABC transporter by at least 50, 60, 70, 80, 90 or 100% in a competition assay.
Embodiment 50. A method of making a chimeric ABC transporter, comprising replacing at least one periplasmic, extracellular, or luminal facing loop and up to 50% of the transmembrane segments on either side of the loop of a parental ABC transporter with equivalent regions of a different ABC transporter.
Embodiment 51. The method of embodiment 50, wherein at least one periplasmic, extracellular, or luminal facing loop and 50% of the transmembrane segments on either side of the loop of the parental ABC transporter are replaced with equivalent regions of the different ABC transporter.
Embodiment 52. The method of embodiment 50, wherein at least one periplasmic, extracellular, or luminal facing loop and up to 25% of the transmembrane segments on either side of the loop of the parental ABC transporter are replaced with equivalent regions of the different ABC transporter.
Embodiment 53. The method of embodiment 50, wherein at least one periplasmic, extracellular, or luminal facing loop and up to 10% of the transmembrane segments on either side of the loop of the parental ABC transporter are replaced with equivalent regions of the different ABC transporter.
Embodiment 54. The method of embodiment 50, wherein at least two, at least three, or all of the periplasmic, extracellular, or luminal facing loops and up to 50% of the transmembrane segments on either side of each loop of the parental ABC transporter are replaced with equivalent regions of the different ABC transporter.
Embodiment 55. The method of any one of embodiments 50-54, wherein the parental and the different ABC transporters are each Type IV or Type V ABC transporters.
Embodiment 56. The method of any one of embodiments 50-55, wherein the parental and the different ABC transporters are each Type IV ABC transporters.
Embodiment 57. The method of any one of embodiments 50-56, wherein the parental ABC transporter is from a Gram-negative bacteria.
Embodiment 58. The method of embodiment 57, wherein the Gram-negative bacteria is selected from Escherichia coli, Enterobacter cloacae, Klebsiella pneumoniae, Pseudomonas aeruginosa, Pseudomonas psychrotolerans, Candidatus accumulibacter, Janthinobacterium agaricidamnosum, Thiomicrospira cyclica, or Magnetospira strain-QH-2.
Embodiment 59. The method of embodiment 58, wherein the Gram-negative bacteria is Escherichia coli.
Embodiment 60. The method of any one of embodiments 57-59, wherein the parental ABC transporter is MsbA.
Embodiment 61. The method of any one of embodiments 57-60, wherein the different ABC transporter is from a Gram-negative bacteria.
Embodiment 62. The method of embodiment 61, wherein the different ABC transporter is from a bacteria selected from Escherichia coli, Enterobacter cloacae, Klebsiella pneumoniae, Pseudomonas aeruginosa, Pseudomonas psychrotolerans, Candidatus accumulibacter, Janthinobacterium agaricidamnosum, Thiomicrospira cyclica, or Magnetospira strain-QH-2.
Embodiment 63. The method of any one of embodiments 50-62, wherein the parental ABC transporter and the different ABC transporter are respectively encoded by homologous genes from two different species.
Embodiment 64. The method of embodiment 63, wherein the parental and different ABC transporters are MsbA transporters from two different Gram-negative bacterial species.
Embodiment 65. A chimeric ABC transporter in which at least one periplasmic, extracellular, or luminal facing loop and up to 50% of the transmembrane segments on either side of the loop of a parental ABC transporter are replaced with equivalent regions of a different ABC transporter.
Embodiment 66. The chimeric ABC transporter of embodiment 65, wherein at least one periplasmic, extracellular, or luminal facing loop and 50% of the transmembrane segments on either side of the loop of the parental ABC transporter are replaced with equivalent regions of a different ABC transporter.
Embodiment 67. The chimeric ABC transporter of embodiment 65, wherein at least one periplasmic, extracellular, or luminal facing loop and up to 25% of the transmembrane segments on either side of the loop of the parental ABC transporter are replaced with equivalent regions of a different ABC transporter.
Embodiment 68. The chimeric ABC transporter of embodiment 65, wherein at least one periplasmic, extracellular, or luminal facing loop and up to 10% of the transmembrane segments on either side of the loop of the parental ABC transporter are replaced with equivalent regions of a different ABC transporter.
Embodiment 69. The chimeric ABC transporter of embodiment 65, wherein at least two, at least three, or all of the periplasmic, extracellular, or luminal facing loops and up to 50% of the transmembrane segments on either side of each loop of the parental ABC transporter are replaced with equivalent regions of a different ABC transporter.
Embodiment 70. The chimeric ABC transporter of any one of embodiments 65-69, wherein the parental ABC transporter is a from a Gram-negative bacteria.
Embodiment 71. The chimeric ABC transporter of embodiment 70, wherein the Gram-negative bacteria is selected from Escherichia coli, Enterobacter cloacae, Klebsiella pneumoniae, Pseudomonas aeruginosa, Pseudomonas psychrotolerans, Candidatus accumulibacter, Janthinobacterium agaricidamnosum, Thiomicrospira cyclica, or Magnetospira strain-QH-2.
Embodiment 72. The chimeric ABC transporter of embodiment 71, wherein the parental ABC transporter is from Escherichia coli.
Embodiment 73. The chimeric ABC transporter of any one of embodiments 70-72, wherein the parental ABC transporter is MsbA.
Embodiment 74. The chimeric ABC transporter of any one of embodiments 70-73, wherein the different ABC transporter is from a Gram-negative bacteria.
Embodiment 75. The chimeric ABC transporter of embodiment 74, wherein the Gram-negative bacteria is selected from Escherichia coli, Enterobacter cloacae, Klebsiella pneumoniae, Pseudomonas aeruginosa, Pseudomonas psychrotolerans, Candidatus accumulibacter, Janthinobacterium agaricidamnosum, Thiomicrospira cyclica, or Magnetospira strain-QH-2.
Embodiment 76. The chimeric ABC transporter of any one of embodiments 70-75, wherein the different ABC transporter is MsbA.
Embodiment 77. The chimeric ABC transporter of any one of embodiments 65-76, wherein the parental ABC transporter and the different ABC transporter are respectively encoded by homologous genes from two different species.
Embodiment 78. The chimeric ABC transporter of embodiment 77, wherein the parental and different ABC transporters are MsbA transporters from two different Gram-negative bacterial species.
Embodiment 79. A chimeric ABC transporter comprising a parental ABC transporter E. coli MsbA (EcMsbA) in which one or more of EcMsbA residues Leu47-Pro68 in periplasmic loop 1 (L1), EcMsbA residues Met159-Leu171 in periplasmic loop 3 (L3), and EcMsbA residues Ala262-Ile292 in periplasmic loop 5 (L5) are replaced with equivalent regions of Pseudomonas psychrotolerans (PpMsbA).
Embodiment 80. A chimeric ABC transporter comprising EcMsbA in which periplasmic loop 1 (L1, EcMsbA residues Leu47-Pro68), periplasmic loop 3 (L3, EcMsbA residues Met159-Leu171), and periplasmic loop 5 (L5, EcMsbA residues Ala262-Ile292) are replaced with equivalent regions of Candidatus accumulibacter (CaMsbA).
Embodiment 81. A chimeric ABC transporter comprising EcMsbA in which one or more of EcMsbA residues Leu47-Pro68 in periplasmic loop 1 (L1), EcMsbA residues Met159-Leu171 in periplasmic loop 3 (L3), and EcMsbA residues Ala262-Ile292 in periplasmic loop 5 (L5) are replaced with equivalent regions of Janthinobacterium agaricidamnosum (JaMsbA).
Embodiment 82. A chimeric ABC transporter comprising EcMsbA in which one or more of EcMsbA residues Leu47-Pro68 in periplasmic loop 1 (L1), EcMsbA residues Met159-Leu171 in periplasmic loop 3 (L3), and EcMsbA residues Ala262-Ile292 in periplasmic loop 5 (L5) are replaced with equivalent regions of Thiomicrospira cyclica (TcMsbA).
Embodiment 83. A chimeric ABC transporter comprising EcMsbA in which one or more of EcMsbA residues Leu47-Pro68 in periplasmic loop 1 (L1), EcMsbA residues Met159-Leu171 in periplasmic loop 3 (L3), and EcMsbA residues Ala262-Ile292 in periplasmic loop 5 (L5) are replaced with equivalent regions of Magnetospira strain-QH-2 (MqMsbA).
Embodiment 84. A molecular complex comprising a chimeric ABC transporter of any one of embodiments 65-83 bound to a peptide, small molecule, antibody, binding fragment of a peptide, binding fragment of a small molecule, or binding fragment of an antibody.
Embodiment 85. The complex of embodiment 84, wherein the peptide is a macrocycle.
Embodiment 86. The complex of embodiment 85, wherein the macrocycle is an 6-14-mer macrocycle.
Embodiment 87. A molecular complex comprising a parental ABC transporter and a molecule of any one of embodiments 27 to 49.
Embodiment 88. The complex of embodiment 87, wherein the peptide is a macrocycle.
Embodiment 89. The complex of embodiment 88, wherein the macrocycle is an 6-14-mer macrocycle.
Embodiment 90. The complex of embodiment 89, wherein the macrocycle is G1118, G1119, G1122, or G1365.
Embodiment 91. A kit comprising the chimeric ABC transporter of any one of embodiments 65-83 and reagents for carrying out the methods of any one of embodiments 1-26, optionally wherein the chimeric ABC transporter is attached to a matrix or beads, and optionally wherein the kit further comprises one or more of the following:
   a. a parental ABC transporter and/or a different ABC transporter from which the chimeric ABC transporter is engineered;
   b. a matrix or beads for attachment of ABC transporters, optionally streptavidin-coated beads, avidin-coated beads, or deglycosylated-avidin-coated beads, or magnetic beads;
   c. one or more detergents for solubilizing an ABC transporter on a matrix or beads;
   d. at least one wash buffer;
   e. at least one elution buffer;
   f. at least one positive or negative control molecule.
Embodiment 92. The kit of embodiment 91, further comprising instructions for use.
Embodiment 93. A method of treating a bacterial infection in an individual comprising administering to the individual an effective amount of a molecule of any one of embodiments 27 to 49.
Embodiment 94. Use of the molecule of any one of embodiments 27 to 49 for treating a bacterial infection in a subject.
Embodiment 95. A peptide comprising the following sequence:

(SEQ ID NO: 17)
ClacF-X1-X2-L-X3-X4-D-X5-X6-X7-X8-MeF-V-C,

wherein:
i. X1 is W, V, or Y;
ii. X2 is W or Y;
iii. X3 is W or Y;
iv. X4 is S, D, V, or H;
v. X5 is N, wherein N is chosen from any natural amino acid other than C, or a non-natural amino acid chosen from Bph ((S)-3-([1,1′-biphenyl]-4-yl)-2-aminopropanoic acid), Dopa (L-3,4-dihydroxyphenylalanine), MeF (N-methyl-L-phenylalanine), and MeG (N-methyl-L-glycine);
vi. X6 is Y, K, A, S, D, R, or V;
vii. X7 is W, Y, or Bph; and
viii. X8 is W or Y;
   optionally wherein the peptide further comprises a G residue following the C residue at the C-terminal end, and wherein ClacF is N-chloroacetyl L-phenylalanine, Bph is (S)-3-([1,1′-biphenyl]-4-yl)-2-aminopropanoic acid, Dopa is L-3,4-dihydroxyphenylalanine, MeF is N-methyl-L-phenylalanine, and MeG is N-methyl-L-glycine.
Embodiment 96. The peptide of embodiment 95, wherein X1 is W or Y.
Embodiment 97. The peptide of embodiment 96, wherein X1 is W.
Embodiment 98. The peptide of any one of embodiments 95-97, wherein X2 is W.
Embodiment 99. The peptide of any one of embodiments 95-98, wherein X3 is W.
Embodiment 100. The peptide of any one of embodiments 95-99, wherein X4 is S, D, V, or H.
Embodiment 101. The peptide of embodiment 100, wherein X4 is D.
Embodiment 102. The peptide of any one of embodiments 95-101, wherein X5 is V, D, H, G, or Y.
Embodiment 103. The peptide of embodiment 102, wherein X5 is V or H.
Embodiment 104. The peptide of embodiment 103, wherein X5 is V.
Embodiment 105. The peptide of any one of embodiments 95-104, wherein X6 is D or S.
Embodiment 106. The peptide of embodiment 105, wherein X6 is S.
Embodiment 107. The peptide of any one of embodiments 95-106, wherein X7 is W.
Embodiment 108. The peptide of any one of embodiments 95-107, wherein X8 is W.
Embodiment 109. A macrocycle formed from the peptide of any one of embodiments 95-108, wherein the macrocycle cyclizes due to a thioether linkage between the N-terminal chloroacetyl group of ClacF and the sulfhydryl group of the C residue.
Embodiment 110. A peptide comprising the following amino acid sequence:

(SEQ ID NO: 19)
ClacF-X1-Y-Bph-MeF-X2-V-C,

wherein:
i. X1 is V, S, Y, W, Dopa, L, V, A, R, K, or D; and
ii. X2 is R, V, Dopa, or Y;
   wherein ClacF is N-chloroacetyl L-phenylalanine, Bph is (S)-3-([1,1′-biphenyl]-4-yl)-2-aminopropanoic acid, Dopa is L-3,4-dihydroxyphenylalanine, and MeF is N-methyl-L-phenylalanine.
Embodiment 111. The peptide of embodiment 110, wherein X1 is S, Y, L, V, A, R, K, or D.
Embodiment 112. The peptide of embodiment 111, wherein X1 is V or Y.
Embodiment 113. The peptide of any one of embodiments 110-112, wherein X1 is V.
Embodiment 114. The peptide of any one of embodiments 110-113, wherein X2 is R or Y.
Embodiment 115. The peptide of embodiment 114, wherein X2 is R.
Embodiment 116. A macrocycle formed from the peptide of any one of embodiments 110-115, wherein the macrocycle cyclizes due to a thioether linkage between the N-terminal chloroacetyl group of ClacF and the sulfhydryl group of the C residue.
Embodiment 117. The peptide or macrocycle of any one of embodiments 95-116, wherein the peptide or macrocycle is conjugated to another molecule, such as an antibiotic or antimicrobial, optionally wherein the conjugation is at the C-terminal amino acid residue of the sequence.
Embodiment 118. The peptide or macrocycle of embodiment 117, wherein the antibiotic or antimicrobial is a polymyxin, such as polymyxin B or polymyxin E.

### BRIEF DESCRIPTION OF THE DRAWINGS

FIGS. 1A-C show diagrams of the protein structure of three different ABC transporters and how the proteins are situated in a membrane relative to the periplasm/extracellular space and the cytoplasm/cytosol. Each individual amino acid is represented by a circle containing the amino acid identified by one-letter code. Some amino acid positions on the chain are also numbered, e.g., position 50 and position 60. FIG. 1A shows the MsbA ABC transporter from Escherichia coli. FIG. 1B shows the human ABC transporter ABCD4. It has ˜25-30% sequence identity with MsbA. FIG. 1C shows the human ABC transporter ABCC1. It has ˜25-30% sequence identity with MsbA. FIGS. 1A-C show and label the periplasmic or extracellular loop regions that may be substituted for an equivalent region of a different ABC transporter to make a chimeric ABC transporter. Also shown are the regions of the proteins that cross the membrane (“the transmembrane segments” or “TM segments”) that may be substituted for an equivalent region of an ABC transporter from a different ABC transporter. The rectangular boxes indicate the regions that may be substituted in each protein.

FIGS. 2A-C show three-dimensional representations of ABC transporters and chimeric ABC transporters and how they are situated in a membrane relative to the periplasm/extracellular space and the cytoplasm/cytosol. FIG. 2A, left panel, shows a three-dimensional representation of the Gram-negative bacterial MsbA situated in a cell membrane (dark band intersecting the structure). FIG. 2A, center panel, shows a three-dimensional representation of a macrocycle-MsbA complex, in which the macrocycle binds to the periplasmic face of the protein, specifically in the periplasmic cleft. FIG. 2A, right panel, shows a chimeric MsbA (MsbA-chimera5), where the darker, filled amino acids at the top of the ribbon diagram are those taken from an equivalent region in a different ABC transporter. Such a chimera may be used, for example, to help select for molecules that bind to the periplasmic face, including the periplasmic cleft, of MsbA, such as the macrocycle shown in the center panel. FIG. 2B, center panel, shows a chimeric human ABCD4 transporter (PDB: 6JBJ), where the filled amino acids of the protein are those taken from an equivalent region in a different ABC transporter. The left panel of FIG. 2B again shows the chimeric MsbA protein, indicating the similarity in the architecture of the two proteins. FIG. 2B, right panel, shows a chimeric human ABCC1 transporter (PDB: 6BHU), where the filled amino acids at the top of the ribbon diagram are those taken from an equivalent region in a different ABC transporter. FIG. 2C shows a representation of each of the three chimeric proteins from a periplasmic or extracellular perspective. Each periplasmic or extracellular loop is labeled. The filled amino acids in the ribbon diagram are those from the equivalent region in a different ABC transporter. Each extracellular loop is also labeled.

FIGS. 3A-N show the strategy for characterizing macrocyclic MsbA inhibitors. FIG. 3A shows a schematic of the INSITE screening strategy for identifying macrocycles targeting the periplasmic face of EcMsbA. FIG. 3B shows exemplary macrocycle inhibitors G1118 and G1365. These inhibitors contain a thioether bond as indicated (non-natural amino acids: Clac-F, N-chloroacetyl L-phenylalanine; MeF, N-Methyl-L-phenylalanine; Bph, (S)-3-([1,1′-biphenyl]-4-yl)-2-aminopropanoic acid. FIGS. 3C-D show dose-response curves of G1118 (FIG. 3C) and G1365 (FIG. 3D) on WT E. coli MsbA and MsbA-chim5. IC₅₀ values were determined by fitting a nonlinear four-parameter inhibition model (see Example 3); data are mean±s.e.m. from three independent experiments. FIG. 3E shows representative electron micrographs comparing msbA⁺, msbA⁻ and inhibitor-exposed E. coli CFT073 UPEC lptD(imp4213) cells (Table 1). G907 is a quinoline inhibitor of MsbA described in FIG. 11D (Alexander et al., 2018; Ho et al., 2018). Inner membrane elaborations are marked with arrows. Images are representative of >10 isolated cells for each condition. Scale bars, 0.1 μm. FIG. 3F shows electron micrographs comparing an E. coli imp strain with G1118 (right panel; arrows mark inner membrane elaborations) and without (left panel). The IC₅₀ of G1118 was determined to be 5 nM. FIGS. 3G-L show dose response curves of G1325 (FIG. 3G; IC₅₀=1.2 μM; EC₅₀=10 μM), G1330 (FIG. 3H; IC₅₀=13 μM; EC₅₀=30 μM), G1365 (FIG. 3I; IC₅₀=300 nM; EC₅₀=20 μM), G1349 (FIG. 3J; IC₅₀=50 μM; EC₅₀=30 μM), G1323 (FIG. 3K; IC₅₀=2.8 μM), and G1320 (FIG. 3L; IC₅₀=185 μM) on E. coli strains imp MsbA_{WT}, imp MsbA^{High}, and UPEC WT. FIGS. 3M-N show potential binding sites for G092 quinoline (FIG. 3M) and G1118 (FIG. 3N).

FIGS. 4A-N show G1118 and G1365 binding in the periplasmic cleft of MsbA. FIG. 4A shows a cryo-EM map of G1118-EcMsbA complex. G1118 and LPS are indicated on the map. FIG. 4B shows a view of G1118 binding in the periplasmic cleft of MsbA. The GKK-tail is omitted for clarity. FIGS. 4C-D show closeup views of select interactions between G1118 and MsbA. FIG. 4E shows a frequency plot of observed macrocycle cDNA in the G1118 family during sequencing after the final round of enrichment (non-natural amino acids are: MeF=F*, N-Methyl-L-phenylalanine; MeG=G*, N-Methyl-L-glycine). FIG. 4F shows a cryo-EM map of G1365-EcMsbA complex. G1365 is indicated. FIGS. 4G-H show closeup views of select interactions between G1365 and MsbA. FIG. 4I shows a frequency plot of observed macrocycle cDNA in the G1365 family during sequencing after the final round of enrichment (non-natural amino acids are: Bph=B*, (S)-3-([1,1′-biphenyl]-4-yl)-2-aminopropanoic acid; MeF=F*, N-Methyl-L-phenylalanine). FIGS. 4J-N show G1365 binds near the membrane exposed region of the outward-facing cleft.

FIGS. 5A-E show LPS bound at an essential peripheral binding site in the outward-facing conformation. FIG. 5A shows a peripheral LPS binding site on MsbA on the inner leaflet of the inner membrane. FIG. 5B shows a closeup view of LPS binding site highlighting interactions between LPS and MsbA. The 2′-hydroxymyristate (2′-C14) and 2″-laurate acyl (2″-C12) chains are labeled. The Kdo residues are omitted for clarity. FIG. 5C shows growth curves of E. coli MG1655 msbA-cKO lptD(imp4213) expressing the indicated WT or mutant alleles of E. coli MsbA from a low-copy plasmid (Table 1). Data are mean±s.e.m. from three independent experiments. FIG. 5D shows a representative thin-section electron micrographs comparing msbA⁺, msbA⁻ and cells expressing the indicated msbA mutant constructs in the E. coli CFT073 UPEC lptD(imp4213) strain. Inner membrane elaborations are shown with arrows. Images are representative of >10 isolated cells for each variant. Scale bars, 0.1 μm. FIG. 5E shows amino acids that cause E. coli growth defects when mutated.

FIG. 6 shows a model for selective recognition and transport of LPS by MsbA. Panel 1—In the outward-facing conformation, LPS can bind the peripheral binding sites along the inner membrane. Mature Kdo2-LipidA is selectively bound by positively charged and aromatic residues on the 4′-phosphate side of LPS. Panel 2—MsbA returns to the inward-facing conformation and LPS is enriched locally upon release from the peripheral binding site (PDB: 6BL6). Facilitated diffusion of LPS towards the central cavity occurs along a ridge of positively charged residues on MsbA. Panel 3—LPS becomes enclosed within the central vestibule, as previously described (PDB: 5TV4). Panel 4—ATP binding causes a large conformational change resulting in the outward-facing state that releases LPS into the outer leaflet of the inner membrane. The LPS diffuses away from MsbA for subsequent processing and transport to the outer membrane.

FIG. 7 shows crystallographic data collection and refinement statistics.

FIG. 8 shows a summary of cryo-EM data acquisition parameters and model refinement statistics.

FIGS. 9A-B show a multi-sequence alignment of select MsbA homologs. In FIG. 9A, E. coli MsbA sequence is shown as the reference, and putative homologs from other species selected from chimeric transporter designs are included. In FIG. 9A, SEQ ID NO: 4 is E. coli MsbA protein sequence (first row), SEQ ID NO: 5 is P. psychrotolerans MsbA protein sequence (second row), SEQ ID NO: 6 is C. accumulibacter MsbA protein sequence (third row), SEQ ID NO: 7 is J. agaricidamnosum MsbA protein sequence (fourth row), SEQ ID NO: 8 is T. cyclica MsbA protein sequence (fifth row), and SEQ ID NO: 9 is Magnetospira. QH-2 MsbA protein sequence (sixth row). FIG. 9B shows overall sequence identities for the MsbA homologs in FIG. 9A.

FIGS. 10A-B show chimeric MsbA transporter constructs. FIG. 10A shows exemplary sequences of E. coli MsbA-based chimeras. Regions marked “loop 1,” “loop 2,” and “loop 3” indicate the periplasmic regions of the transmembrane helices and loops that were substituted in generating the engineered chimeric transporter proteins. In FIG. 10A, SEQ ID NO: 4 is E. coli MsbA protein sequence (first row), SEQ ID NO: 12 is E. coli MsbA-chimera1 protein sequence (second row), SEQ ID NO: 13 is E. coli MsbA-chimera2 protein sequence (third row), SEQ ID NO: 14 is E. coli MsbA-chimera3 protein sequence (fourth row), SEQ ID NO: 15 is E. coli MsbA-chimera4 protein sequence (fifth row), and SEQ ID NO: 16 is E. coli MsbA-chimera5 protein sequence (sixth row). FIG. 10B shows an outward-facing E. coli MsbA and MsbA-chimera. Side views are shown in the top panel and top views are shown in the bottom panel. In the MsbA-chimera, the regions with amino acid substitutions at loops 1-3 (as shown in FIG. 10A) are indicated.

FIGS. 11A-E show purification and evaluation of EcMsbA chimeras. FIG. 11A shows SDS-PAGE analysis of purified EcMsbA chimeras, visualized with Coomassie brilliant blue staining. FIG. 11B shows an overlay of size exclusion chromatography profiles with UV (280 nm) curves shown. FIG. 11C shows a comparison of ATPase activity of decreasing concentrations of the EcMsbA chimeras, compared to WT protein. FIG. 11D shows exemplary chemical structures of quinoline and benzophenone inhibitors (Alexander et al., 2018; Ho et al., 2018). FIG. 11E shows dose-response curves of compounds on purified EcMsbA and EcMsbA-chim5. Data are mean±s.e.m. from three independent experiments (FIGS. 11C-E). IC₅₀ values (FIG. 11E, in parentheses) were determined by fitting the inhibition dose-response curve with a nonlinear four-parameter inhibition model (Example 3).

FIGS. 12A-B show the crystal structure of MsbA-chimera5 which reveals putative benzophenone receptor site. FIG. 12A shows the overall structure of MsbA-chim5 in an inward-facing conformation. LPS is shown in spheres. The assigned benzophenone G758 is shown as sticks. Fo-Fc map (1.5 σ, mesh) is calculated before G758 was included in the model and refinement. FIG. 12B shows a close-up view of the putative benzophenone binding site on MsbA with the Fo-Fc map from FIG. 12A, and the 2Fo-Fc map (1.5 σ, mesh). G758 putatively binds within a shallow, hydrophobic pocket around residues identified through frequency of resistance-mapping studies (data not shown).

FIGS. 13A-D show evaluation of the biochemical and phenotypic activity of MsbA macrocycle inhibitors. FIG. 13A shows dose-response curves of G1118 on purified and amphipol-reconstituted MsbA homologues from E. coli, E. cloacae, K. pneumoniae and P. aeruginosa. FIG. 13B shows dose-response curves of G1118 on E. coli CFT073 (WT) and CFT073 lptD(imp4213) (imp) strains. FIG. 13C shows dose-response curves of G1365 on purified and amphipol reconstituted MsbA homologues from E. coli, E. cloacae, K. pneumoniae and P. aeruginosa. FIG. 13D shows dose-response curves of G1118 on E. coli CFT073 (WT) and CFT073 lptD(imp4213) (imp) strains. Data are mean±s.e.m. from three independent experiments (FIGS. 13A-D). IC₅₀ values (in parentheses) were determined by fitting the inhibition dose-response curve with a nonlinear four-parameter inhibition model (Example 3).

FIGS. 14A-G show biochemical evaluation of complexes used in structure studies. FIG. 14A shows chemical structures of G1118 and G1365 derivatives used in the structure studies and for isolation of the PD-1365 resistant mutant (G1365·I2,Dopa3). Regions of the derivatives that differ from the G1118 or G1365 parent macrocycles, i.e., KK, R2, I2,Dopa3, and L2^{G4S2GEE}, are indicated with circles. FIGS. 14B-F shows dose-response curves of G1118 and G1365 parent and derivative macrocycles on purified EcMsbA. FIG. 14G shows comparison of ATPase activity of increasing concentrations of EcMsbA in the presence or absence of Fab 12G7. Data are mean±s.e.m. from three independent experiments (FIGS. 14B-G). IC₅₀ values (FIG. 14B-F, in parentheses) were determined by fitting the inhibition dose-response curve with a nonlinear four-parameter inhibition model (Example 3).

FIGS. 15A-E show cryo-EM data processing for MsbA in complex with G1118. FIG. 15A shows the processing pipeline. FIG. 15B shows a local resolution plot calculated in Relion. FIG. 15C shows orientation distribution of all particles from the final round of 3D-refinement. FIG. 15D shows an FSC plot from refinement in cisTEM. Maximum resolution used for alignment was 4.5 Å. FIG. 15E shows selected densities from cryo-EM map.

FIGS. 16A-D show macrocycle binding in the periplasmic cleft of MsbA. In FIGS. 16A-B, the electrostatic surface of MsbA bound to G1118 highlights the complex chemical environment of the macrocycle binding site. Approximate membrane boundary and the non-enforced C2 symmetry axis are labeled. FIGS. 16C-D show the electrostatic surface of MsbA bound to G1365.

FIGS. 17A-E show cryo-EM data processing for MsbA in complex with G1365. FIG. 17A shows the processing pipeline. FIG. 17B shows orientation distribution of all particles from the final round of 3D-refinement. FIG. 17C shows an FSC plot from refinement in cisTEM. Maximum resolution used for alignment was 4.5 Å. FIG. 17D shows local resolution plot calculated in RELION. FIG. 17E shows selected densities from cryo-EM map.

FIGS. 18A-D show the conserved peripheral LPS binding site. FIG. 18A shows the MsbA-G1118-LPS complex with symmetrically related LPS binding sites. FIG. 18B shows a sequence conservation analysis of EcMsbA. FIG. 18C shows electrostatic potential on the surface of MsbA in the inward-facing, LPS-bound conformation (PDB: 5TV4). A line of positive charge run is present from the LPS binding site towards the central cavity. Missing side-chain atoms were added through the Protein Preparation Wizard in Maestro. The binding position of LPS in the outward-facing G1118-bound structure is shown. In FIG. 18D, the structure of MsbA-G1118-LPS (blue) is aligned with inward-facing MsbA (white, PDB: 5TV4). Residues within 15 Å of LPS were used for alignment.

FIGS. 19A-J show evaluation of the cell growth phenotype of MsbA peripheral LPS binding site mutants. FIG. 19A shows α-FLAG-MsbA and α-GroEL western blots of solubilized extracts from E. coli MG1655 msbA-cKO lptD)(imp4213) expressing WT or the indicated mutants of E. coli MsbA from a pLMG18 vector, in the presence of 2% arabinose (under these conditions, untagged WT FcMsbA is also expressed). The blots are representative of n=2 independent experiments. FIGS. 19B-I show growth curves of E. coli MG1655 msbA-cKO lptD(imp4213) expressing the indicated WT or mutant alleles of E. coli MsbA, or a no plasmid control, in the presence or absence of arabinose. 2% arabinose was used to induce expression of WT E. coli MsbA from the chromosome, whereas in the absence of arabinose, the only source of MsbA is constitutive expression from the pLMG18 vector expressing WT or the indicated mutants of E. coli MsbA. FIG. 19J shows comparison of ATPase activity of decreasing concentrations of the EcMsbA mutants, compared to WT protein. Data (FIGS. 19B-J) are mean±s.e.m. from three independent experiments.

### DETAILED DESCRIPTION

### I. Definitions

Unless otherwise defined, scientific and technical terms used in connection with the present invention shall have the meanings that are commonly understood by those of ordinary skill in the art. As utilized in accordance with the present disclosure, the following terms, unless otherwise indicated, shall be understood to have the following meanings:

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As used herein, the transition term “consisting essentially of,” when referring to steps of a claimed process signifies that the process comprises no additional steps beyond those specified that would materially affect the basic and novel characteristics of the process. As used herein, the transition term “consisting essentially of,” when referring to a composition or product, such as a kit, signifies that it comprises no additional components beyond those specified that would materially affect its basic and novel characteristics.

As used herein, the singular forms “a,” “and,” and “the” include plural referents unless the context clearly dictates otherwise. Thus, for example, reference to “an ABC transporter protein” includes a plurality of such transporter proteins, and reference to “the cell” includes reference to one or more cells (or to a plurality of cells) and equivalents thereof known to those skilled in the art, and so forth.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

An “ABC transporter” as used herein refers to an ATP binding cassette (“ABC”) transporter. ABC transporters constitute a superfamily of integral membrane proteins found in prokaryotes and eukaryotes that are responsible for the ATP-powered translocation of many substrates across membranes, from small inorganic and organic molecules, such as amino acids, sugars, nucleosides, vitamins, and metal clusters, to larger organic compounds, including peptides, lipid molecules, oligonucleotides, and polysaccharides. ABC transporters can generally be grouped into “exporters” and “importers,” although some ABC transporters may fall into another group, nontransporter ABC proteins.

ABC transporters have a characteristic architecture, generally including at least four domains: two transmembrane domains (TMDs) embedded in the membrane and two nucleotide binding domains (NBDs). ATP hydrolysis on the NBDs drives conformational changes in the TMDs, resulting in alternating access from the inner side and outer side of the membrane for one-way transport of substrates across the membrane. Each TMD is made up of transmembrane alpha-helices; each TMD typically has 6-10 transmembrane alpha-helices, with most exporters having 6 transmembrane alpha-helices per TMD. The NBDs are highly conserved. In contrast, the TMDs that create the translocation pathway are more variable, for example, depending on the substrate the ABC transporter is transporting.

One classification system described in Thomas et al., “Structural and functional diversity calls for a new classification of ABC transporters,” 594 FEBS Letters 3767-3775 (2020) (incorporated by reference in its entirety for its description of an ABC transporter classification system), groups ABC transporters into seven distinct types, I-VII, based on their TMD fold. This nomenclature system can be universally applied and is based on ABC transporter structural information determined by sequence analysis, homology modeling, X-ray crystallography and single-particle cryo-electron microscopy, among other information. Type I transporters have the following transmembrane helix organization: (5-6)+(5-6/8). Type II transporters have the following transmembrane helix organization: 10+10. Type III transporters have the following transmembrane helix organization: 4-8 (T)+6-7 (S). Type IV transporters have the following transmembrane helix organization: 6+6. Type V transporters also have a 6+6 transmembrane helix organization and are further defined as of the ABCG/ABCA/Wzm type on the basis of sequence similarity and known substrate specificity. Type VI transporters also have a 6+6 transmembrane helix organization and are further defined as of the LptB2FG type on the basis of distinct structural features. Type VII transporters have the following transmembrane helix organization: 4+4. Thus, as used herein, a “Type IV” ABC transporter, for example, refers to an ABC transporter that would be classified under Type IV under this classification system, a “Type V” refers to a Type V transporter under this system, etc.

The terms “outward-facing conformation” and “inward-facing conformation” as used herein refer to conformations of the alpha-helices of the two transmembrane domains or regions (TMDs) of ABC transporters, which are packed in such a way that they form a transmembrane pore that is either accessible from the inner area of a membrane (inward-facing) or from the outside of a membrane (outward-facing). In a Gram-negative bacterial cell, for instance, an ABC transporter integrated into the inner membrane has an inward-facing conformation in which the pore opens to the cytoplasm and an outward-facing conformation in which the pore opens to the periplasm (the space between the inner membrane and the outer membrane). In a Gram-positive bacterial cell or in a eukaryotic cell, an ABC transporter integrated into the cell membrane has an inward-facing conformation in which the pore opens to the cytoplasm and an outward-facing conformation in which the pore opens to the outside of the cell. In an ABC transporter integrated into the membranes of subcellular organelles, such as mitochondria, the ABC transporter has an outward-facing conformation in which the pore opens to the lumen of the subcellular organelle and inward-facing conformation in which the pore opens to the cytoplasm/cytosol. Thus, the outward-facing conformation is one in which the pore opens to the lumen of the organelle.

The term “periplasmic, extracellular, and/or luminal face” of an ABC transporter refers to the face or region of the ABC transporter that is accessible when the transporter is in an outward-facing conformation, depending on the membrane in which the transporter is located. The “periplasmic, extracellular, and/or luminal cleft” refers to a pocket or pore formed by the packing of alpha-helices that is accessible in the outward-facing conformation of the protein. In some cases, the outward portion of the ABC transporter faces the periplasm, e.g., in a Gram-negative bacterial ABC transporter, while in other cases it faces the exterior of a cell (e.g., extracellular) or it faces into a lumen, where the ABC transporter is in a subcellular organelle (e.g., luminal). Therefore, a “periplasmic cleft” as used herein refers to a pocket or pore in an ABC transporter, formed by the packing of the alpha-helices of the transmembrane domains, that is accessible to the periplasm. An “extracellular cleft” as used herein refers to a pocket or pore in an ABC transporter, formed by the packing of the alpha-helices of the transmembrane domains, that is accessible to the environment outside the cell. A “luminal cleft” as used herein refers to a pocket or pore in an ABC transporter, formed by the packing of the alpha-helices of the transmembrane domains, that is accessible to the lumen of a subcellular organelle, such as a mitochondria, endoplasmic reticulum, Golgi, etc.

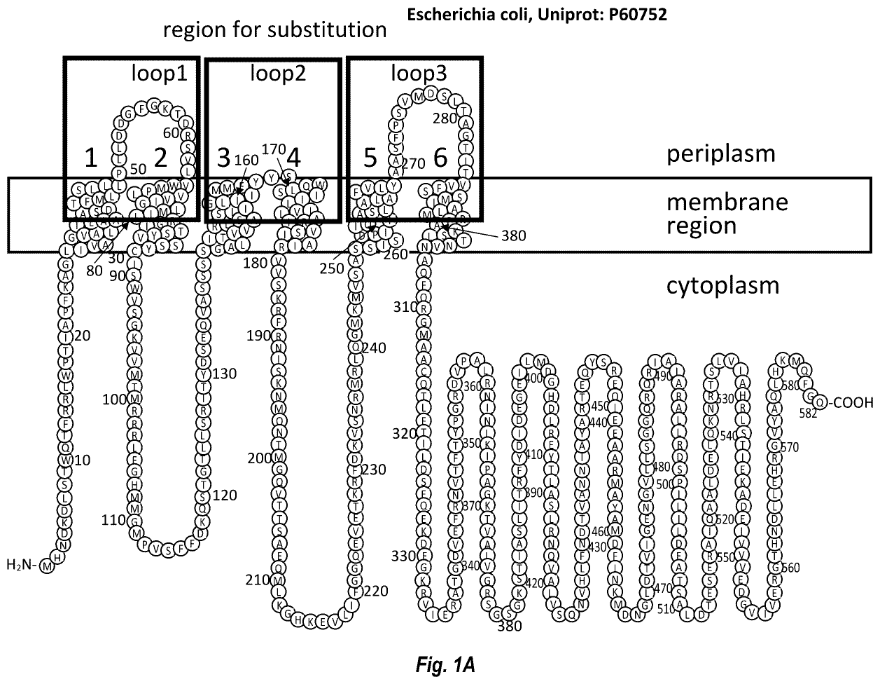

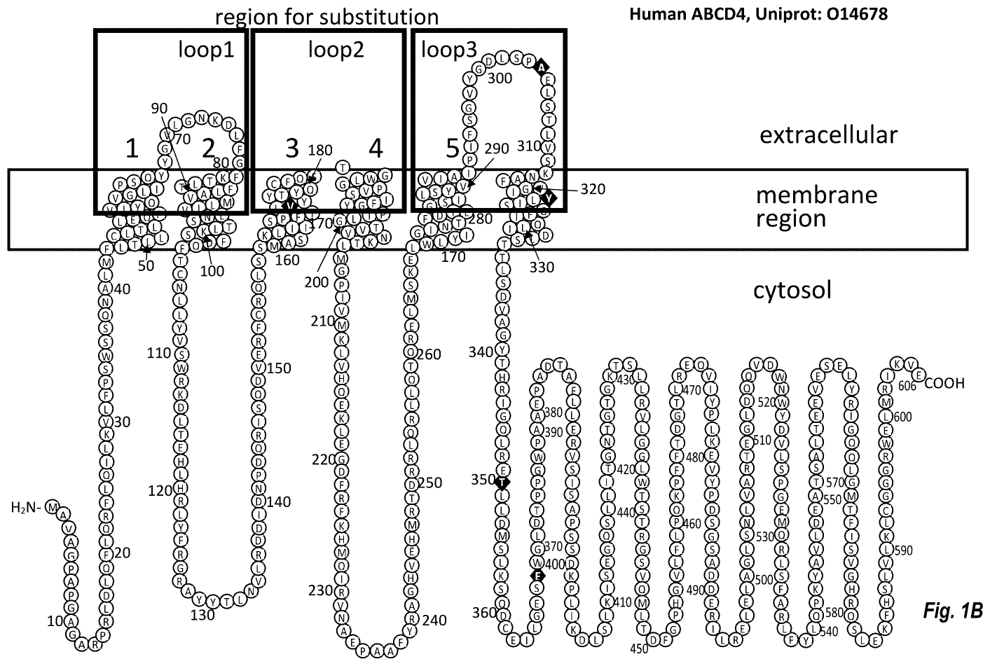

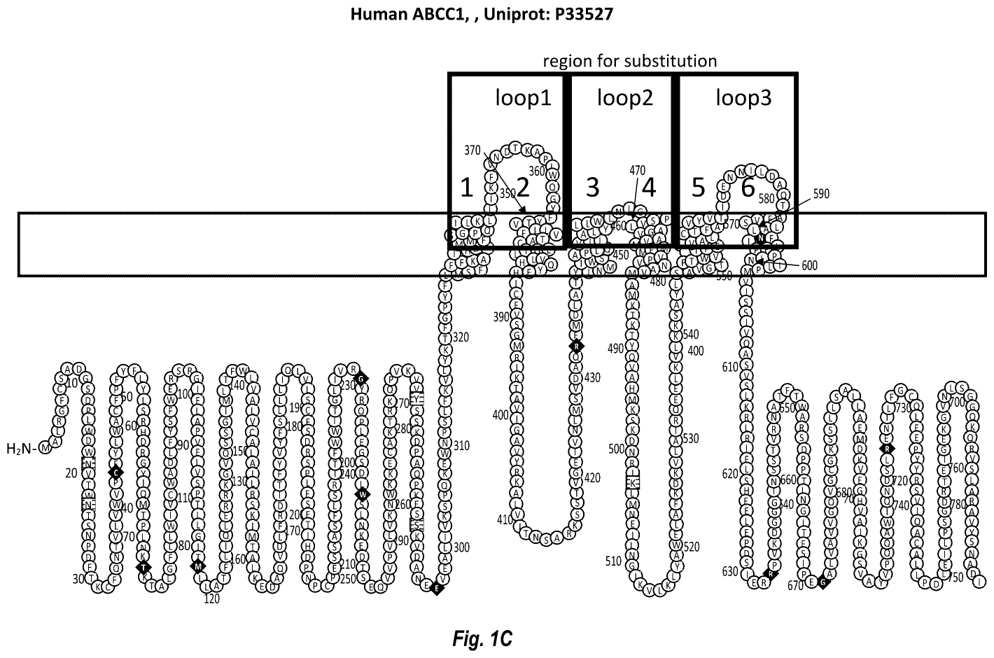

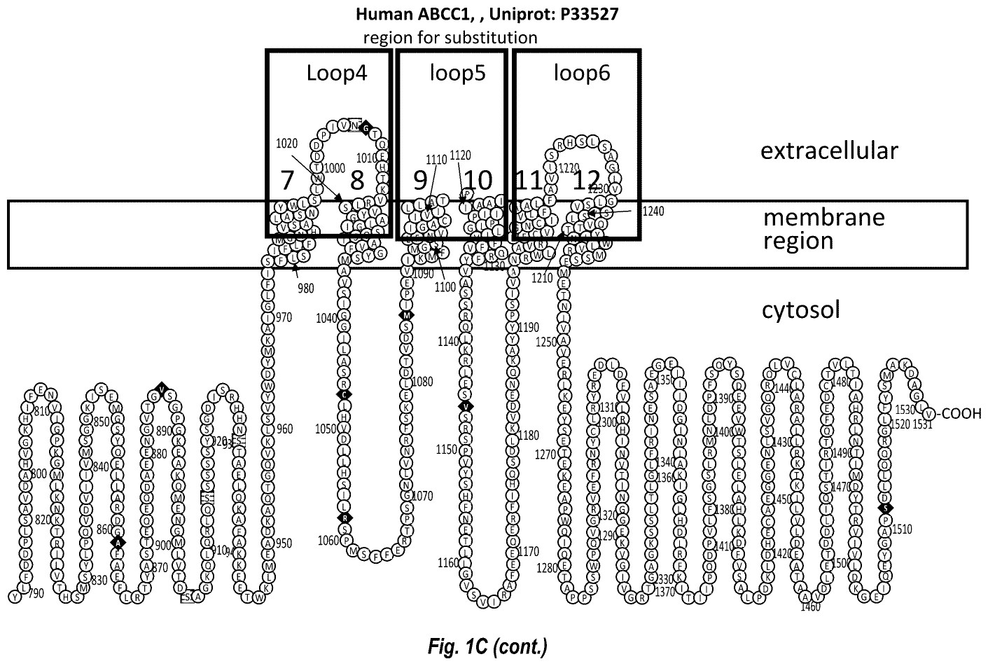

A “chimeric ABC transporter” as used herein refers to an ABC transporter in which one or more of the periplasmic, extracellular, or luminal facing loops and up to 50% of the transmembrane segments on either side of the loop or loops in question are replaced with equivalent regions of a different ABC transporter. As an example, the diagrams in FIGS. 1A-B show three periplasmic (or extracellular or luminal) facing loops, each of which connects two alpha helices that cross the membrane. As used herein, the “equivalent regions” (also termed “corresponding regions”) being inserted from the different ABC transporter are those that in the same location within the protein when folded as the residues being removed from the parental ABC transporter. In some cases, the regions in the ABC transporter to remove and replace with regions from the chimeric ABC transporter may be determined using sequence alignments and structural information for the two proteins.

In some cases, a chimeric ABC transporter is formed from a parental ABC transporter and a different ABC transporter from “homologous genes from different species.” As used herein, this phrase means that the two genes are members of the same gene family, and may also transport the same molecules, such as representing MsbA proteins from two different bacterial species such as E. coli and another Gram-negative bacterial species.

The term “Gram-negative bacteria” as used herein refers to bacteria that do not retain crystal violet dye when the Gram staining method is employed. The Gram stain is a common method for general bacterial identification. In the Gram stain method, bacteria may be heat fixed on a slide, stained with crystal violet dye, flushed with iodine, decolorized with alcohol or another organic solvent, and then counterstained with safranin. The Gram reaction reflects fundamental differences in the biochemical and structural properties of bacteria. Gram-positive bacteria remain purple because they have a single thick cell wall that is not easily penetrated by the solvent. Gram-negative bacteria are decolorized because they have cell walls with much thinner layers that allow removal of the dye by the solvent. In the final step, the safranin stains the Gram-negative cells red.

The term “Gram-positive bacteria” as used herein refers to bacteria that retain crystal violet dye when the Gram staining method is employed. The Gram stain is a common method for general bacterial identification. In the Gram stain method, bacteria may be heat fixed on a slide, stained with crystal violet dye, flushed with iodine, decolorized with alcohol or another organic solvent, and then counterstained with safranin. The Gram reaction reflects fundamental differences in the biochemical and structural properties of bacteria. Gram-positive bacteria remain purple because they have a single thick cell wall that is not easily penetrated by the solvent. Gram-negative bacteria are decolorized because they have cell walls with much thinner layers that allow removal of the dye by the solvent. In the final step, the safranin stains the Gram-negative cells red.

The term “peptide” as used herein refers to a chain of fifty amino acids or less linked by peptide bonds, including amino acid chains of 2 to 50, 2 to 15, 2 to 10, 2 to 8, or 6 to 14 amino acids.

The term “small molecule” as used herein refers to an organic molecule having a molecular weight of 50 Daltons to 2500 Daltons.

The term “macrocycle” or “macrocylic molecule” as used herein refers to a cyclic macromolecule or a macromolecular cyclic portion of a macromolecule. Macrocycles range in size from 500 Daltons to 2000 Daltons. In some cases herein, macrocycles are cyclic peptides or peptide derivatives.

The term “binding fragment” as used herein refers to a portion of a larger molecule, such as a small molecule, peptide, or antibody, that is expected to directly contact the ABC transporter. Binding fragments may be used in high-throughput screens.

In this disclosure, “binds” or “binding” or “specific binding” and similar terms, when referring to a molecule that “binds” to an ABC transporter protein or chimeric ABC transporter protein, for example, means that the binding affinity is sufficiently strong that the interaction between the members of the binding pair cannot be due to random molecular associations (i.e. “nonspecific binding”). Thus, the binding is selective or specific. Such binding typically requires a dissociation constant (K_{D}) of 100 μM or less (i.e., corresponding to an affinity of 100 μM or greater), and may often involve a K_{D} of 20 μM or less, 10 μM or less, 1 μM or less, or 500 nM or less.

The term “competition assay” as used herein refers to an assay in which a molecule being tested prevents or inhibits specific binding of a reference molecule to a common target.

The term “ATPase assay” refers to an assay used to measure the degree of conversion of ATP to ADP by a protein, such as an ABC transporter protein.

The term “treat,” as well as words stemming therefrom, as used herein, does not necessarily imply 100% or complete treatment. Rather, there are varying degrees of treatment, including, for example, reducing at least one symptoms or of a condition, in some cases, for example, about 100%, about 90%, about 80%, about 70%, about 60%, about 50%, about 40%, about 30%, about 20%, or about 10%. Furthermore, treatment also includes prevention, amelioration, or inhibition of one or more conditions or symptoms of a disorder, as well as delaying the onset of the disorder, or a symptom thereof.

The terms “effective amount” or “therapeutically effective amount,” as used herein, refer to a sufficient amount of a molecule disclosed herein being administered which will relieve to some extent one or more of the symptoms of the disease or condition being treated, e.g., an infection.

Other definitions are included in the sections below, as appropriate.

### II. Chimeric ABC Transporters

In some embodiments, the invention comprises chimeric ABC transporters.

In some embodiments, to make a chimeric ABC transporter, the starting material is a specific ABC transporter (a “parental” ABC transporter). In some embodiments, the parental ABC transporter is from a eukaryotic cell. In some embodiments, the parental ABC transporter is a human ABC transporter. In some embodiments, the parental ABC transporter is from a Gram-positive bacteria. In some embodiments, the parental ABC transporter is from a Gram-negative bacteria. In some embodiments, the parental ABC transporter is one that may be embedded in a subcellular organelle membrane. In some embodiments, the parental ABC transporter is a transporter found in a particular bacterial species selected from Escherichia coli, Enterobacter cloacae, Klebsiella pneumoniae, Pseudomonas aeruginosa, Pseudomonas psychrotolerans, Candidatus accumulibacter, Janthinobacterium agaricidamnosum, Thiomicrospira cyclica, or Magnetospira strain-QH-2. In some embodiments, the parental ABC transporter is MsbA, e.g., Escherichia coli MsbA.

In some embodiments, after a parental ABC transporter is selected, an equivalent but different ABC transporter is identified, which in some embodiments is from a different organism or species, from which amino acid sequences or regions will be taken in order to form a chimera with the parental transporter. In some cases, the goal is to identify a different ABC transporter that is sufficiently different in sequence such that a test molecule that binds to the parental ABC transporter will not bind to the different ABC transporter at an equivalent region, but that is otherwise similar enough that the chimera will properly fold into its correct architecture. In some embodiments, one identifies the different ABC transporter by searching for a different ABC transporter that shares 20-99% sequence identity with the parental ABC transporter. In some embodiments, the different ABC transporter is from a homologous gene as the one being tested, but from a different, optionally related, species or organism. For example, the chimera, in some cases, may be constructed from ABC transporters of homologous genes from two different bacterial species or genera, or from human and mouse, or human and primate, etc., species, such as the MsbA proteins from two different bacterial species. In some embodiments, the three-dimensional structure of the parental ABC transporter is compared with the three-dimensional structure of a different ABC transporter, where they are known.

In some embodiments, where the parental ABC transporter is a human protein, the different ABC transporter is from a homologous gene from a different eukaryotic species. In some embodiments, the different ABC transporter is from a homologous gene of a different mammalian species. In some embodiments, where the parental ABC transporter is from a gram-positive bacteria, the different ABC transporter is from another gram-positive bacteria, and is optionally from a homologous gene in that other species. In some embodiments, where the parental ABC transporter is from a Gram-negative bacteria, the different ABC transporter is from a Gram-negative bacteria, and optionally from a homologous gene in that other species. In some embodiments, the parental ABC transporter and the different ABC transporter are selected from two different species selected from: Escherichia coli, Enterobacter cloacae, Klebsiella pneumoniae, Pseudomonas aeruginosa, Pseudomonas psychrotolerans, Candidatus accumulibacter, Janthinobacterium agaricidamnosum, Thiomicrospira cyclica, and Magnetospira strain-QH-2. In some embodiments, the chimeric ABC transporter comprises regions of a parental ABC transporter selected from a eukaryotic cell, a Gram-positive bacteria, a Gram-negative bacteria, Escherichia coli, Enterobacter cloacae, Klebsiella pneumoniae, Pseudomonas aeruginosa, Pseudomonas psychrotolerans, Candidatus accumulibacter, Janthinobacterium agaricidamnosum, Thiomicrospira cyclica, or Magnetospira strain-QH-2, and/or MsbA and regions of a different ABC transporter selected from a eukaryotic cell, a Gram-positive bacteria, a Gram-negative bacteria, Escherichia coli, Enterobacter cloacae, Klebsiella pneumoniae, Pseudomonas aeruginosa, Pseudomonas psychrotolerans, Candidatus accumulibacter, Janthinobacterium agaricidamnosum, Thiomicrospira cyclica, or Magnetospira strain-QH-2, and/or MsbA.

In some embodiments, it may be advantageous to select a different ABC transporter that is the same type of ABC transporter as the parental ABC transporter as defined by the classification system in Thomas et al., “Structural and functional diversity calls for a new classification of ABC transporters,” 594 FEBS Letters 3767-3775 (2020) (incorporated by reference in its entirety for its description of an ABC transporter classification system), which groups ABC transporters into seven distinct types, I-VII, based on their trans-membrane domain (TMD) fold. For example, in some embodiments, the parental ABC transporter is a type I ABC transporter, and the different ABC transporter is a type I ABC transporter. In some embodiments, the parental ABC transporter is a type II ABC transporter, and the different ABC transporter is a type II ABC transporter. In some embodiments, the parental ABC transporter is a type III ABC transporter, and the different ABC transporter is a type III ABC transporter. In some embodiments, the parental ABC transporter is a type IV ABC transporter, and the different ABC transporter is a type IV ABC transporter. In some embodiments, the parental ABC transporter is a type V ABC transporter, and the different ABC transporter is a type V ABC transporter. In some embodiments, the parental ABC transporter is a type VI ABC transporter, and the different ABC transporter is a type VI ABC transporter. In some embodiments, the parental ABC transporter is a type VII ABC transporter, and the different ABC transporter is a type VII ABC transporter.

ABC transporters have a characteristic architecture, generally including at least four domains: two transmembrane domains (TMDs) (transmembrane segments may be portions of TMDs) embedded in the membrane and two nucleotide binding domains (NBDs). Each TMD is made up of transmembrane alpha-helices; each TMD typically has 6-10 transmembrane alpha-helices, with most exporters having 6 transmembrane alpha-helices per TMD. In some embodiments, the ABC transporters have loops that face into the periplasmic, extracellular, or luminal space when the molecule is in the outward-facing conformation, and transmembrane segments that connect to either side of such loops. (See FIGS. 1A-1C.) In some embodiments, the transporter has three such loops. In some embodiments, the transporter has six such loops.

FIG. 1A shows a representation of an ABC transporter from Escherichia coli. The TMDs are the portions of the amino acid chain that cross the membrane region—these are typically alpha-helices. The six TMDs in the representation are labeled 1 through 6. The representation also shows loops on the periplasmic side of the membrane and loops on the cytoplasmic side of the membrane. The loops each have a first end that connects to a TMD, a second end that connects to a TMD, and a portion that reaches out into the periplasm or the cytoplasm. The ABC transporter shown has three loops that face toward the periplasm (loops 1, 2, and 3, which correspond to loops 1, 3, and 5 of MsbA (with loops 2, 4, and 6 of MsbA facing the cytoplasm) for example). The rectangular boxes indicate the regions that may be substituted with an equivalent region from a different ABC transporter, namely at least one of the loops facing toward the periplasm, or all three of the loops, and up to 50% of the TMD segment.

FIG. 1B shows a representation of an ABC transporter from human ABC transporter ABCD4. The six TMDs in the representation are labeled 1 through 6. The representation also shows loops on the extracellular side of the membrane and loops on the cytosol side of the membrane. In the representation, the loops on the extracellular side are labeled 1 through 3. The rectangular boxes indicate the regions that may be substituted with an equivalent region from a different ABC transporter.

FIG. 1C shows a representation of an ABC transporter from human ABC transporter ABCC1. This ABC transporter is structured differently than the ABC transporter from Escherichia coli and human ABCD4, also shown in FIGS. 1A and 1B, but many of the same identifying characteristics are present. For example, there are extracellular loops, but instead of three extracellular loops like in ABCD4, there are six extracellular loops, which are labeled 1 through 6 in the representation. Likewise, there are twelve TMDs, which are labeled 1 through 12 in the representation. The rectangular boxes indicate the regions that may be substituted with an equivalent region from a different ABC transporter.

In each of the three representations in FIGS. 1A-C, the rectangular boxes indicate the regions that may be substituted with an equivalent region from a different ABC transporter. In some embodiments, up to 50%, 0-50%, 10-50%, 25-50%, 0-10%, 0-25%, or 10-25% of the TMD on either side of a loop being replaced in a chimera is also replaced. In some embodiments, the disclosure comprises a chimeric ABC transporter in which at least one periplasmic, extracellular, or luminal facing loop and up to 50% (i.e., 50%, 0-50%, 10-50%, 25-50%, 0-10%, 0-25%, or 10-25%) of the transmembrane segments on either side of the loop are replaced with equivalent regions of a different ABC transporter. In some embodiments, the invention comprises a chimeric ABC transporter in which at least one periplasmic, extracellular, or luminal facing loop and 50% of the transmembrane segments on either side of the loop are replaced with equivalent regions of a different ABC transporter. In some embodiments, the invention comprises a chimeric ABC transporter in which at least one periplasmic, extracellular, or luminal facing loop and up to 25% of the transmembrane segments on either side of the loop are replaced with equivalent regions of a different ABC transporter. In some embodiments, the invention comprises a chimeric ABC transporter in which at least one periplasmic, extracellular, or luminal facing loop and up to 10% of the transmembrane segments on either side of the loop are replaced with equivalent regions of a different ABC transporter. In some embodiments, the invention comprises a chimeric ABC transporter in which wherein two or more, or all periplasmic, extracellular, or luminal facing loops and up to 50% (i.e., 50%, 0-50%, 10-50%, 25-50%, 0-10%, 0-25%, or 10-25%) of the transmembrane segments on either side of each loop are replaced with equivalent regions of a different ABC transporter.

In some embodiments, predictions of the location (i.e., boundaries) of loops and transmembrane segments in the parental ABC transporter and in the different ABC transporter may be made based on available experimental structure templates from the Protein Data Bank (PDB) when available; or, alternatively, by using the closest available PDB structural template and standard homology modeling approaches and software (i.e. Swiss-Modell, Phyre2, MOE). In some embodiments, in the absence of a suitable PDB template, predictions of the location (i.e., boundaries) of the loops and transmembrane segments in the ABC transporter and in the different ABC transporter may also be made using standard databases or algorithms (i.e. Uniprot, TMHMM server, etc.).

In some embodiments, once the ABC transporter and the different ABC transporter and the location of at least one loop and/or transmembrane segment in each have been identified, a region of the ABC transporter may be replaced with a region from the different ABC transporter to create a chimeric ABC transporter. In some embodiments, for example, if a region from the ABC transporter is loop 1, an equivalent region from the different ABC transporter is also loop 1. In some embodiments, for example, if a region from the ABC transporter is 25% of the transmembrane segment/TMD 1, an equivalent region from the different ABC transporter is also 25% of the transmembrane segment/TMD 1.

In some embodiments, where the ABC transporter is MsbA, the chimeric ABC transporter comprises EcMsbA (amino acid sequence available at Uniprot P60752) in which one or more of EcMsbA residues Leu47-Pro68 in periplasmic loop 1 (L1), EcMsbA residues Met159-Leu171 in periplasmic loop 3 (L3), and EcMsbA residues Ala262-Ile292 in periplasmic loop 5 (L5) are replaced with equivalent regions of Pseudomonas psychrotolerans (PpMsbA; Uniprot A0A1G5PEL0). (The nomenclature of the loops here considers both periplasmic and cytoplasmic loops, where L1, L3, and L5 face the periplasm and L2, L4, and L6 face the cytoplasm. Thus, L1, L3, and L5 of EcMsbA are equivalent to loops 1, 2, and 3 shown in FIG. 1A.) Sequences are based on the amino acid sequence of the E. coli protein, which has accession number Uniprot P60752 (see www (dot) uniprot (dot) org entry P60752), incorporated by reference herein. See also the Sequence Table (Table 7) herein.

In some embodiments, the chimeric ABC transporter comprises EcMsbA in which periplasmic loop 1 (L1, EcMsbA residues Leu47-Pro68), periplasmic loop 3 (L3, EcMsbA residues Met159-Leu171), and periplasmic loop 5 (L5, EcMsbA residues Ala262-Ile292) are replaced with equivalent regions of Candidatus accumulibacter sp. SK-12 (CaMsbA; Uniprot A0A011NLL4).

In some embodiments, the chimeric ABC transporter comprises FcMsbA in which one or more of EcMsbA residues Leu47-Pro68 in periplasmic loop 1 (L1), EcMsbA residues Met159-Leu171 in periplasmic loop 3 (L3), and FcMsbA residues Ala262-Ile292 in periplasmic loop 5 (L5) are replaced with equivalent regions of Janthinobacterium agaricidamnosum (JaMsbA; Uniprot A0A3G2E7N4).

In some embodiments, the chimeric ABC transporter comprises EcMsbA in which one or more of EcMsbA residues Leu47-Pro68 in periplasmic loop 1 (L1), EcMsbA residues Met159-Leu171 in periplasmic loop 3 (L3), and EcMsbA residues Ala262-Ile292 in periplasmic loop 5 (L5) are replaced with equivalent regions of Thiomicrospira cyclica from strain DSM 14477 (TcMsbA; Uniprot F6DCY0).

In some embodiments, the chimeric ABC transporter comprises EcMsbA residues Leu47-Pro68 in periplasmic loop 1 (L1), EcMsbA residues Met159-Leu171 in periplasmic loop 3 (L3), and FcMsbA residues Ala262-Ile292 in periplasmic loop 5 (L5) are replaced with equivalent regions of Magnetospira sp. strain-QH-2 (MqMsbA; Uniprot W6KCN7).

### III. Screening Methods Using Chimeric ABC Transporters

The present disclosure encompasses, inter alia, methods of identifying molecules that bind to the periplasmic, extracellular, and/or luminal face of a particular ABC transporter protein using a chimeric version of that protein as described above. In some cases, the methods identify molecules that bind to the periplasmic, extracellular, and/or luminal cleft of the protein.

In some embodiments, the methods comprise determining whether a test molecule binds to the periplasmic, extracellular, and/or luminal face of a parental ABC transporter, comprising (a) providing a chimeric ABC transporter, as described above, in which one or more regions of the periplasmic, extracellular, and/or luminal face of the parental ABC transporter are substituted with one or more equivalent regions of the periplasmic, extracellular, and/or luminal face of a different ABC transporter; and (b) contacting the chimeric ABC transporter with a test molecule that binds to the parental ABC transporter in an outward-facing conformation, wherein the test molecule is determined to bind to the periplasmic, extracellular, and/or luminal face of the parental ABC transporter if the test molecule does not bind to the chimeric ABC transporter.

In some cases, a molecule is first tested to determine whether it binds to the parental ABC transporter in the outward-facing conformation, e.g., by (a) trapping the parental ABC transporter in the outward-facing conformation and then (b) selecting a test molecule that binds to the parental ABC transporter in the outward-facing conformation before completing the above steps. Thus, after this selection, the method comprises (c) providing a chimeric ABC transporter, as described above, in which one or more regions of the periplasmic, extracellular, and/or luminal face of the parental ABC transporter are substituted with one or more equivalent regions of the periplasmic, extracellular, and/or luminal face of a different ABC transporter; and (d) contacting the chimeric ABC transporter with the test molecule of (b) that binds to the parental ABC transporter in an outward-facing conformation, wherein the test molecule is determined to bind to the periplasmic, extracellular, and/or luminal face of the ABC transporter if the test molecule does not bind to the chimeric ABC transporter. In either of these methods, in some embodiments, the methods further comprise trapping the chimeric ABC transporter in an outward-facing conformation prior to contacting the chimeric ABC transporter with the test molecule. In the methods above, the ABC transporter and/or the chimeric ABC transporter may be trapped in an outward-facing conformation by treating the ABC transporter and/or the chimeric ABC transporter with Mg²⁺, ATP, and vanadate (e.g., a vanadate or orthovanadate anion).

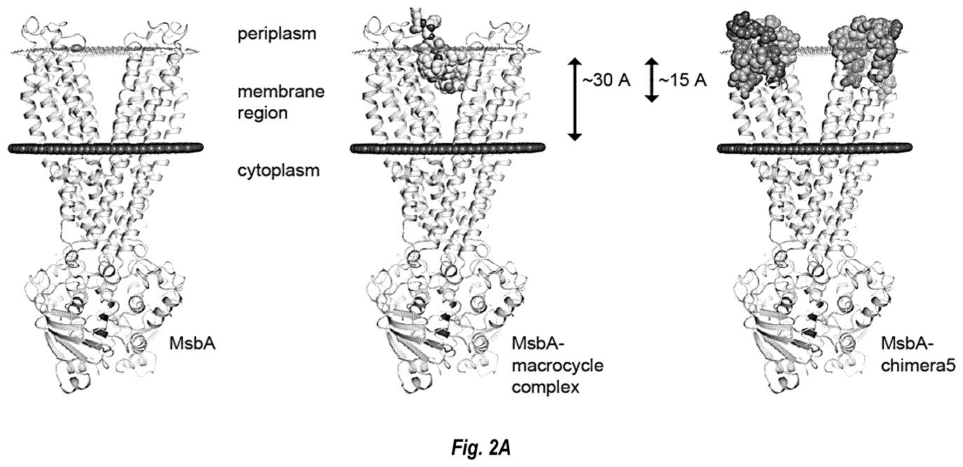

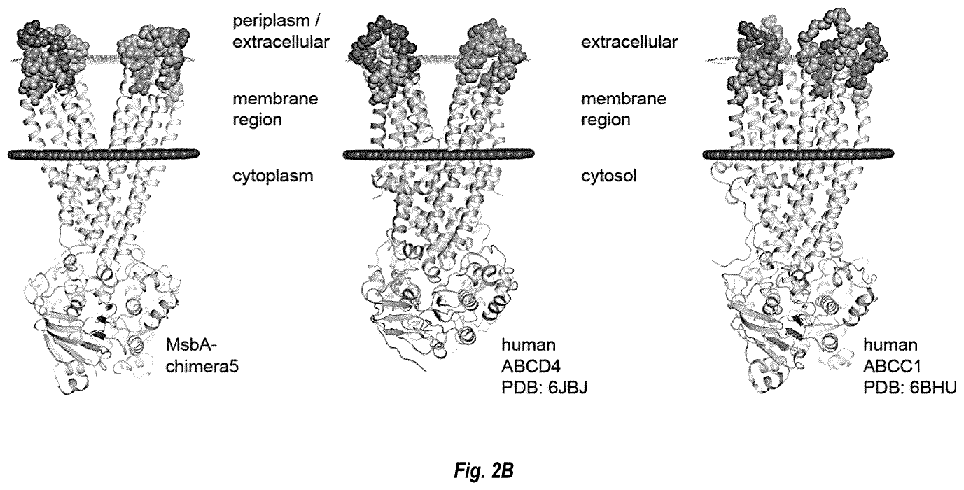

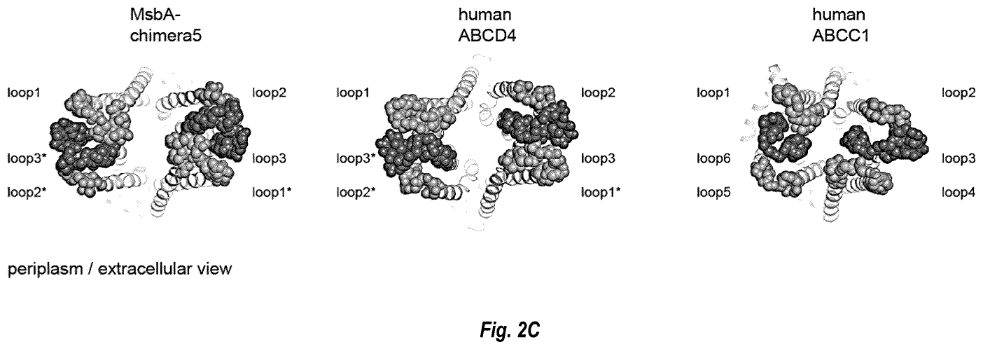

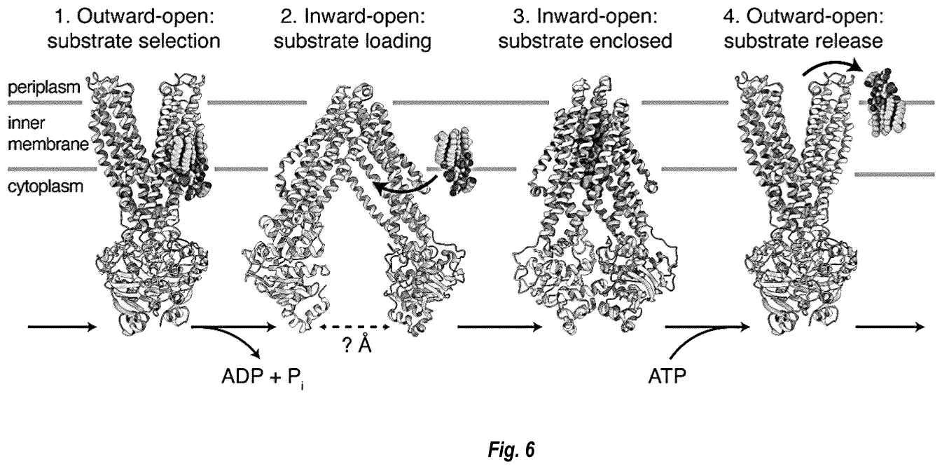

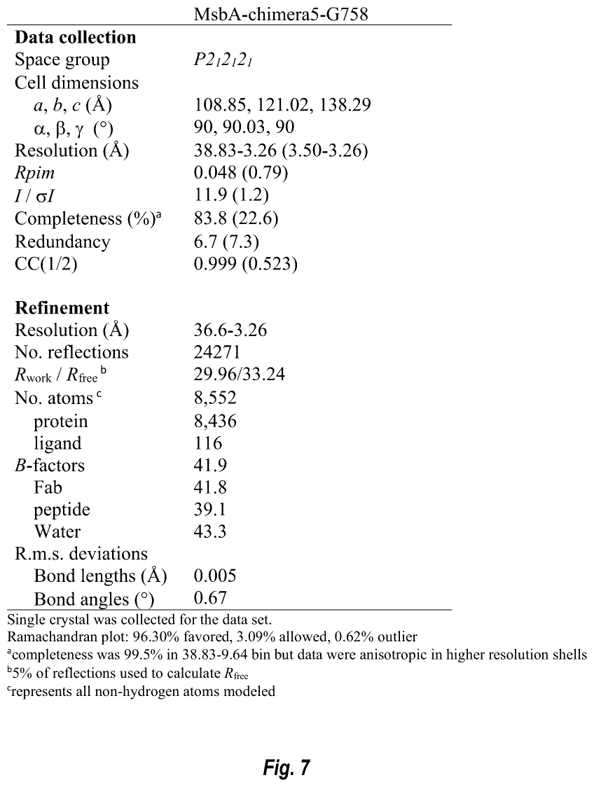

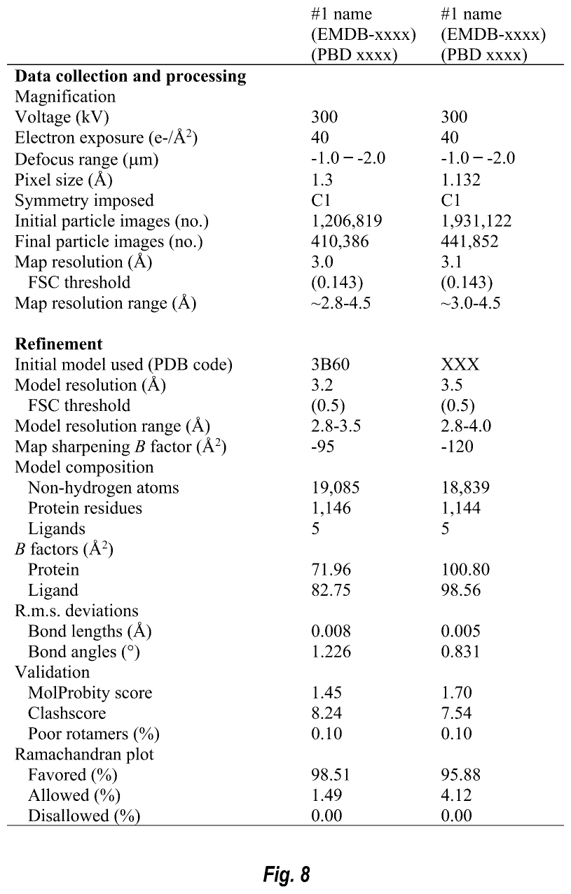

These basic steps are also illustrated, for example, in FIG. 6. As depicted in FIG. 6, ABC transporters have both an inward-facing and an outward-facing conformation. Previously identified ABC transporter binders such as those in the quinoline class bind to the inward-facing conformation, as shown in FIG. 6 in the second panel form the left. As described herein, the transporter can also be trapped in an outward-facing conformation by treatment with agents such as a combination of Mg²⁺, ADP, and vanadate, thus exposing the periplasmic, extracellular, or luminal face of the protein, and the associated periplasmic, extracellular, or luminal cleft. FIG. 6. To selectively identify molecules that bind to the periplasmic, extracellular, and/or luminal face or its associated cleft, a counter-selection may be performed using the appropriate chimeric ABC transporter described above (FIGS. 1A-1C, 2A-2C, and 6). One may select for molecules that bind to the parental ABC transporter but that do not bind to the chimera under the appropriate assay conditions, e.g., chimera null binders.

In some embodiments, the methods are performed with either the test molecule or the ABC transporter immobilized, such as on a bead or matrix platform. In some cases, the parental ABC transporter and/or chimeric ABC transporter is immobilized on a bead or matrix platform, such as using biotin/streptavidin or a similar set of reagents. Where beads are used for immobilization, they can have any shape, such as flakes or chips, spheres, pellets, etc. A matrix may be comprised of beads or smaller particles, and could be, for example, a slurry or gel, which, in turn, could be placed onto a plate or chip, such as a microwell plate or the like. In some embodiments, the matrix or beads are coated with streptavidin, avidin, or deglycosylated-avidin. In some embodiments, the beads are magnetic beads, for example, to facilitate their collection during an assay by use of magnetic instruments. For example, in some cases, the protein may be biotinylated and then exposed to a matrix or beads coated with streptavidin, thus allowing for the protein to become attached to the matrix or beads.

In certain assays herein, such as those in which the parental and/or chimeric ABC transporter is immobilized while test molecules are in solution, molecules that are determined to bind to the particular transporter may be identified as those that remain bound to the immobilized transporter under the assay conditions following incubation and washing of the immobilized transporter, and thus, that elute from the protein-bound matrix or beads upon addition of elution buffer. Molecules that do not bind to the particular transporter protein in the assay may be identified as those that are not eluted (e.g., in more than trace levels) upon addition of elution buffer, and thus, that are removed from the immobilized protein upon washing the protein-bound matrix or beads.

In some embodiments, the parental ABC transporter or chimeric ABC transporter is solubilized in a detergent or similar molecule that mimics a biological membrane so that it maintains an appropriate fold and ability to form a correct outward-facing conformation. Exemplary detergents or related molecules or systems for solubilizing ABC transporters and/or chimeric ABC transporters include lauryl maltose neopentyl glycol (LMNG), and in some embodiments, 0.02% LMNG, as well as dodecyl-ß-D-maltoside (DDM), brij-35, glycol-diosgenin, digitonin, amphiphols such as amphiphol A8-35, and lipid nanodiscs. For example, detergents may solubilize the transporter to stabilize it, while amphiphols may effectively wrap around the hydrophobic portions of the protein to stabilize them, and the protein may also be stabilized in a type of lipid bilayer created by lipid nanodiscs.

In some embodiments, a library of test molecules is screened. The library may be contacted with a chimeric ABC transporter on a matrix or beads and then washed at least once with a wash buffer to remove non-binding molecules. Bound test molecules are then eluted and analyzed. Molecules that bind preferentially to an ABC transporter in the outward-facing conformation compared to the related chimeric ABC transporter in its outward-facing conformation may be identified as those that bind to the periplasmic, extracellular, and/or luminal face of the ABC transporter, since those regions are mutated in the chimeric ABC transporter.

In some embodiments, further experiments are performed on molecules selected in the above screens, for example, to determine their binding affinity for each of the parental ABC transporter and the chimeric ABC transporter, and to determine how they impact the function of the ABC transporter. Thus, for example, in some embodiments, an ATPase assay is performed to determine the ATP to ADP activity of the ABC transporter in the presence of the identified molecule. In some embodiments, an identified molecule that bind to the periplasmic, extracellular, and/or luminal face of the ABC transporter may act as an inhibitor of the ATPase activity of the ABC transporter. ATPase assays are known in the art and various commercial kits are available, such as the Tanscreener ADP2 Assay (BellBrook Labs, Cat. #3010-1K) and the Molecular Probes ATP Determination Kit (Thermofisher, Cat. #A22066).

In some embodiments, the binding affinity of the identified molecule for the parental ABC transporter and/or the chimeric ABC transporter may be determined. In some cases, this can be done in an ELISA assay similar to that used in the initial screening, to obtain an IC₅₀ value, for example. In some cases, a competition ELISA assay may also be performed, for example, using the parental ABC transporter bound to a matrix or beads and a chimeric ABC transporter free in solution or vice versa. A molecule that binds to the parental ABC transporter in preference over the corresponding chimeric ABC transporter should bind to the parental ABC transporter to a roughly equivalent extent in the presence and in the absence of the chimeric ABC transporter. Such an assay may be performed, for example, to verify that a particular test molecule or a molecule identified in a prior screen is selective for the periplasmic, extracellular, and/or luminal face of the transporter. In some embodiments, a binding assay may be performed in cell culture, to test for binding to the parental ABC transporter in the cell membrane of a cell or otherwise in its normal cellular state. Other types of binding assays are known in the art.

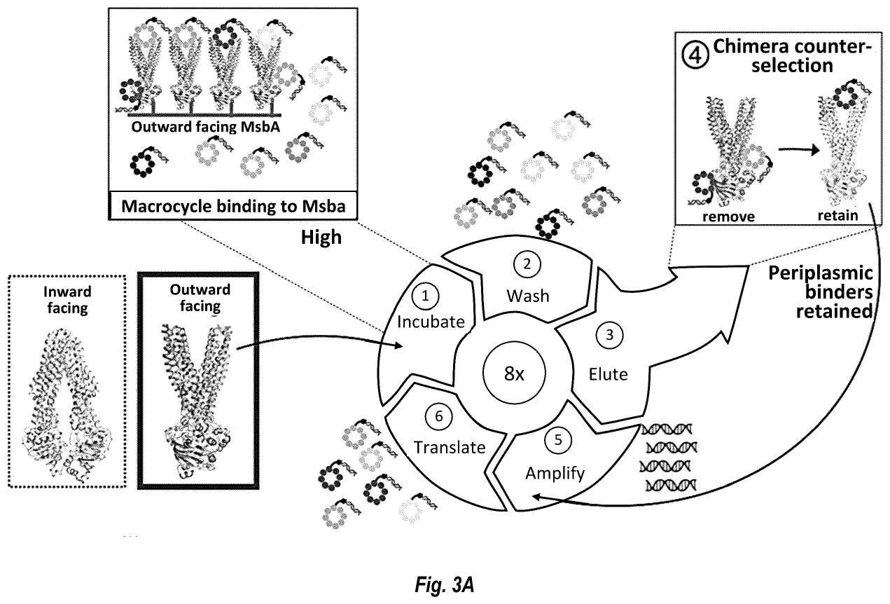

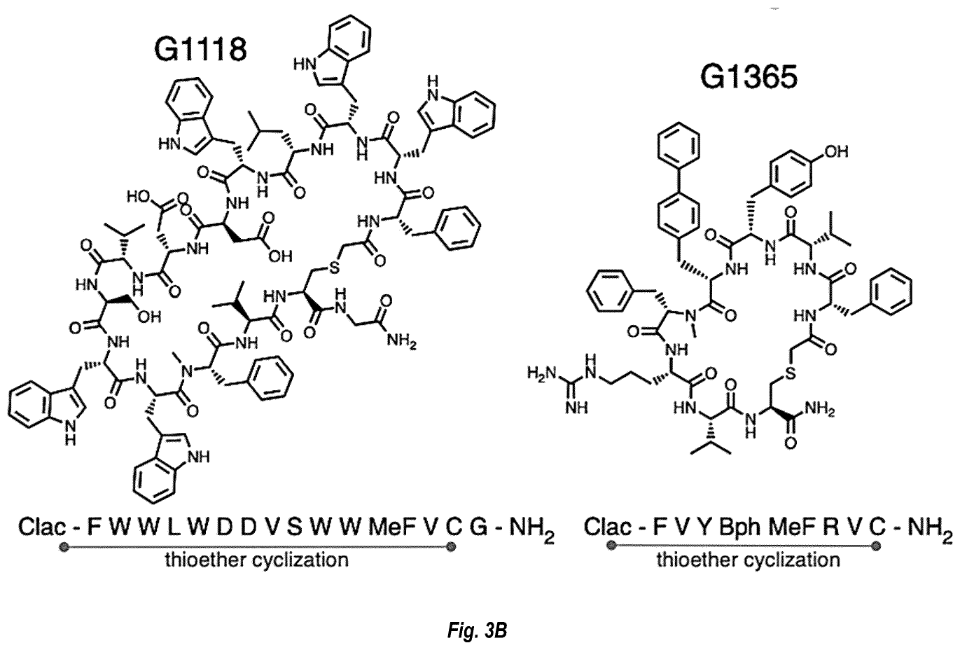

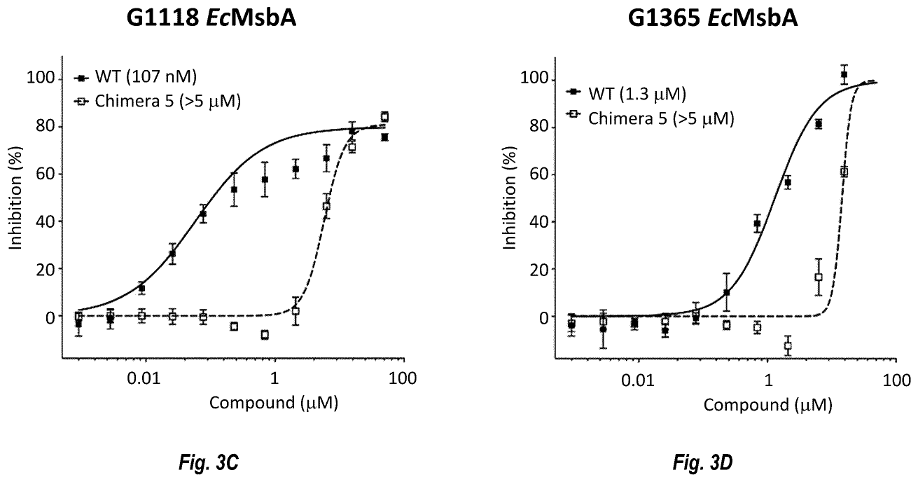

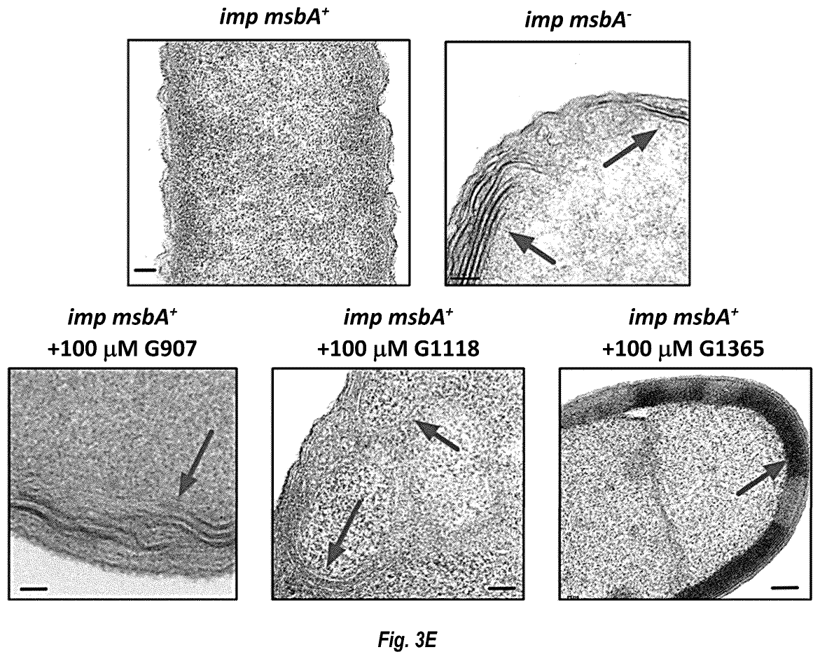

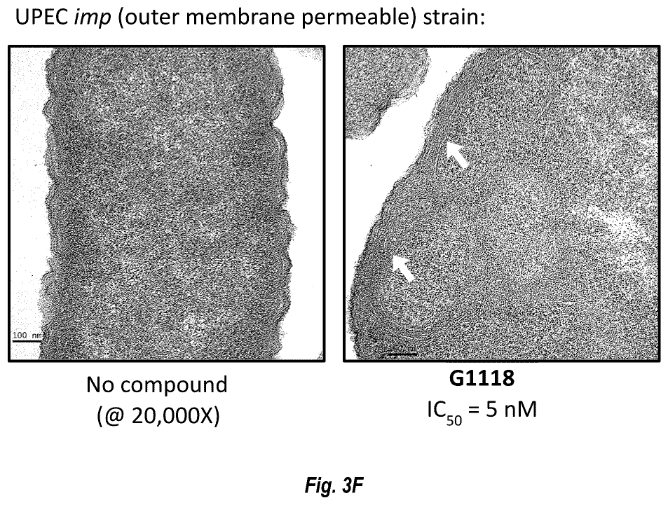

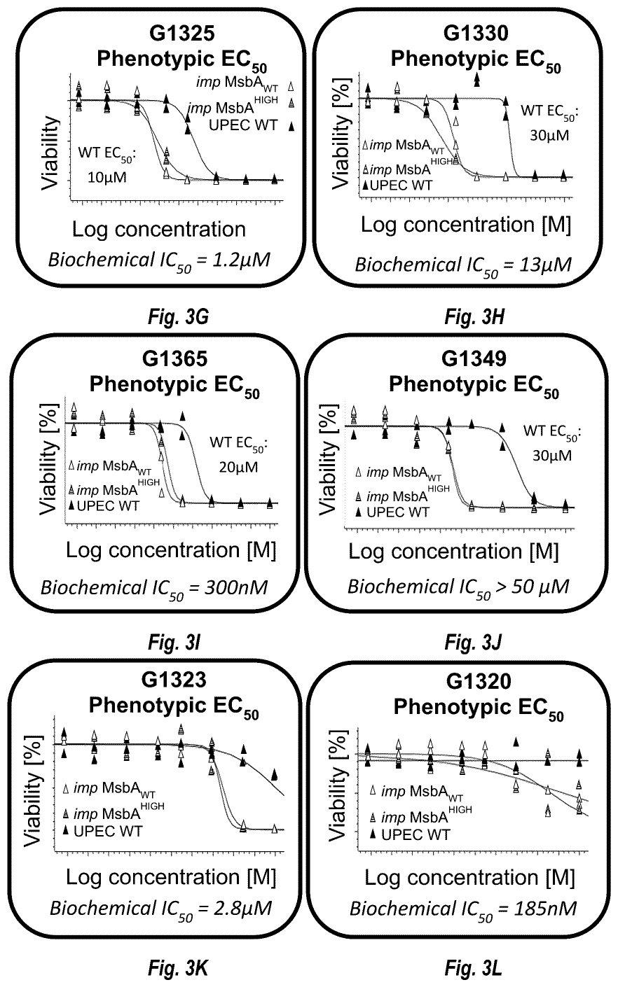

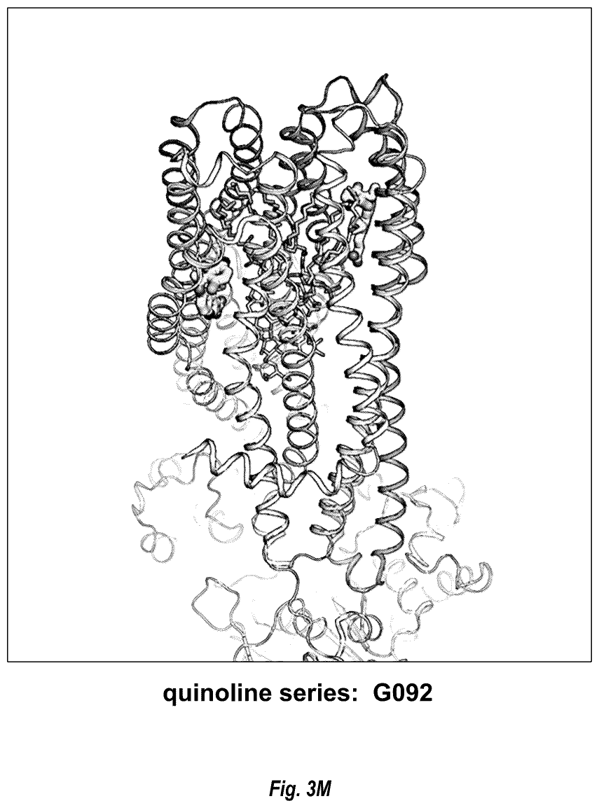

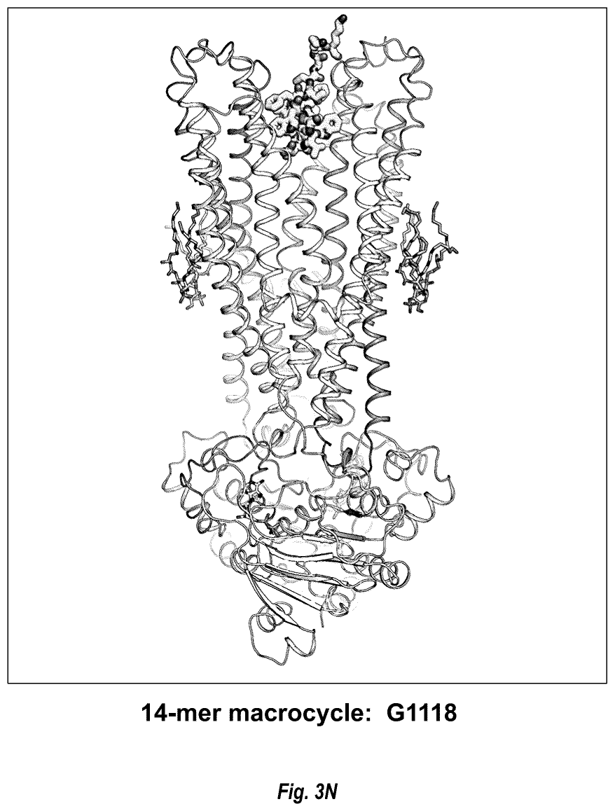

In some embodiments, one or more functional assays may be performed to test the effect of molecules identified in a screen on the function of the parental ABC transporter. For example, in some cases, a molecule that binds to the periplasmic, extracellular, and/or luminal face of an ABC transporter acts as an inhibitor of the transporter. For example, if inhibition of an ABC transporter activity is expected to result in loss of cell viability or reduction of cell growth, for example, then a cell viability or growth assay can be conducted to determine if presence of the identified molecule affects these parameters. In some cases, other assays may be conducted to determine the impact of a molecule on the function of the transporter. In some cases, the transport of the transporter's substrate in the presence and absence of the molecule can be tested. For example, in the case of MsbA, tested in the Examples below, certain binding molecules were found to slow LPS transport by the protein on the basis of electron microscopy (EM) analyses. Lack of LPS transport causes a stacking of inner and outer membranes at the surface of a bacterial cell that is clearly visible by EM. See FIG. 3E for example. Appropriate functional assays for the ABC transporter in question are known or may be readily developed based on knowledge in the art.

### IV. Test Molecules for Screening Methods

In some embodiments, the disclosure comprises screening methods that test particular types of molecules, as well as molecules identified by any of the screening methods described herein as binding to the periplasmic, extracellular, and/or luminal face or cleft of a particular ABC transporter. In some cases, the identified molecules do not bind to the chimeric ABC transporter used in the screening methods, but do bind to the parental ABC transporter on which the chimera is based. In some cases, the identified molecules bind to the parental ABC transporter with an affinity at least 10 fold tighter than to the chimeric ABC transporter used in the screen. In some cases, the identified molecules bind to the parental ABC transporter with an affinity at least 100 fold tighter than to the chimeric ABC transporter used in the screen. In some cases, the identified molecules bind to the parental ABC transporter with an affinity at least 1000 fold tighter than to the chimeric ABC transporter used in the screen.

In some embodiments, the molecule to be tested is a peptide. In some embodiments, the peptide is a 6-14-mer peptide, such as a 6-12-mer, a 6-10-mer, a 6-8-mer, an 8-12-mer, an 8-10-mer, or the like. In some embodiments, the peptide is a 14-mer. See Table 3 and FIG. 3F. In some embodiments, the peptide is an 6-10 mer. In some embodiments, the peptide is an 8-10 mer. In some embodiments, the peptide is an 6-8 mer. In some embodiment, the peptide is an 8-mer. See FIGS. 3E and 3G-L, and Table 5. In some embodiments, the peptide is a 3-40-mer, a 3-20-mer, a 4-16-mer, a 4-14-mer, or a 6-14-mer, such as a 3-mer, 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, or 40-mer.

In some embodiments, the peptide is a macrocycle. In some embodiments, the macrocycle is a 6-14 mer macrocycle, such as a 6-12-mer, a 6-10-mer, a 6-8-mer, an 8-12-mer, an 8-10-mer, or the like. In some embodiments, the macrocycle is a 14-mer macrocycle. See Table 3 and FIG. 3F. In some embodiments, the macrocycle is an 6-10 mer macrocycle. In some embodiments, the macrocycle is an 8-10 mer macrocycle. In some embodiments, the macrocycle is an 6-8 mer macrocycle. In some embodiments, the macrocycle is an 8-mer macrocycle. See FIGS. 3E and 3G-L, and Table 5. In some embodiments, the macrocycle is a 3-40-mer, a 3-20-mer, a 4-16-mer, a 4-14-mer, or a 6-14-mer, such as a 3-mer, 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, or 40-mer macrocycle. In some embodiments, the macrocycle has at least one lipophilic side-chain and at least one positively charged side-chain.

In some embodiments, the molecule to be tested in a screen herein is a small molecule. In some embodiments, the molecule to be tested is an antibody, which may include not only full length antibodies of any of IgG, IgM, IgA, IgD, and IgE, but also an antigen binding fragment of an antibody, such as an Fv, Fab′, (Fab′)2, scFv, or the like, a nanobody, single-chain antibody, bispecific or multispecific antibody.

In some embodiments, the molecule to be tested is a binding fragment of a peptide, a binding fragment of a small molecule, or a binding fragment of an antibody (e.g., an antigen binding fragment).

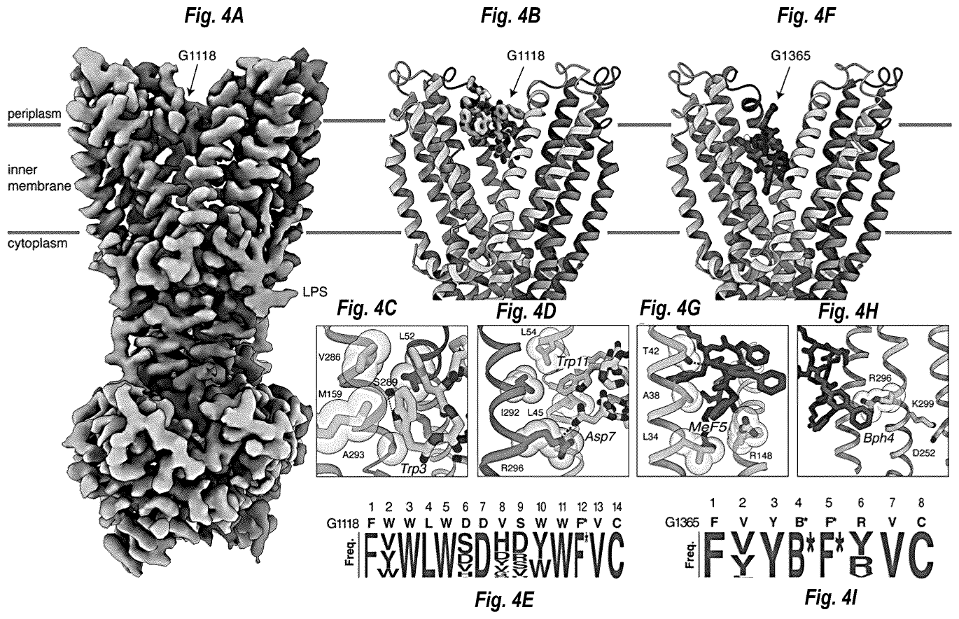

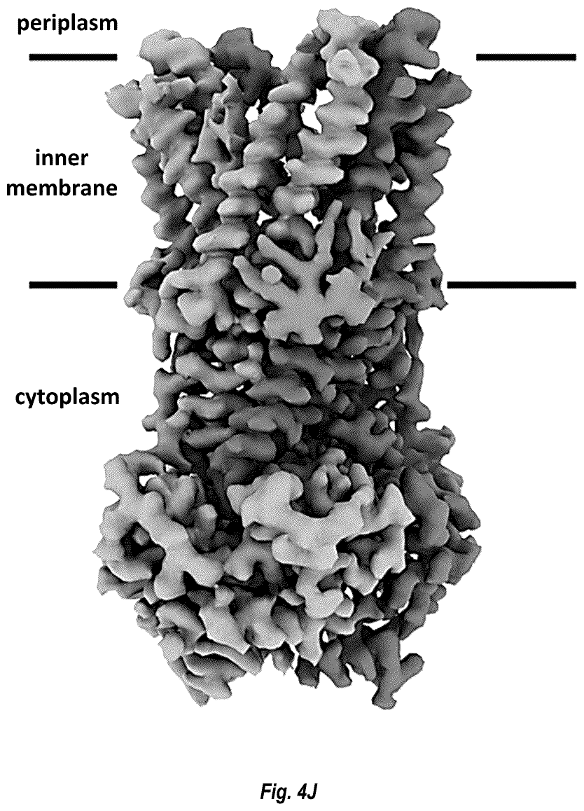

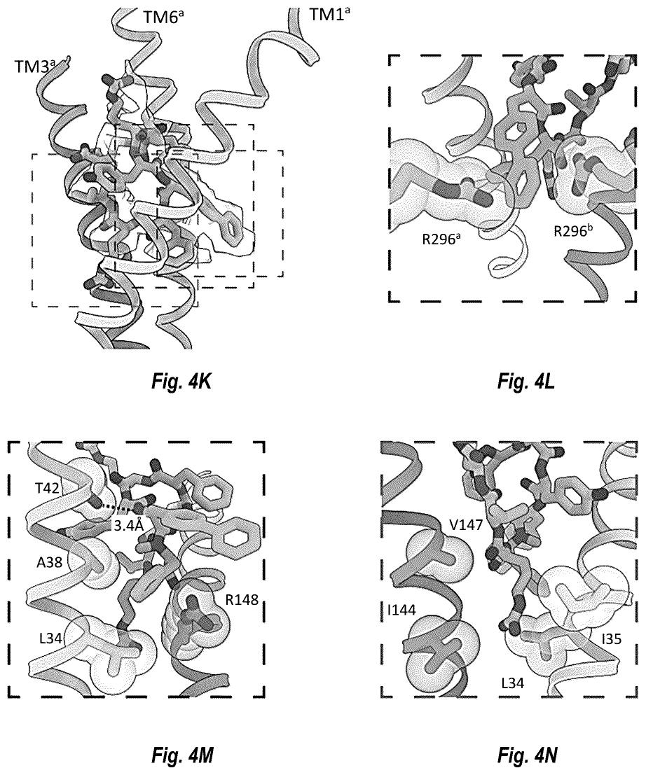

Also encompassed herein are particular macrocycles identified in screens for binders of E. coli MsbA. In some embodiments, the macrocycle is G1118, which is a 14-mer macrocycle. See FIGS. 3F, 4A-D, and 6. G1118 was identified as binding to E. coli MsbA, for example, with an IC₅₀ for binding to MsbA in an imp E. coli strain (in which the outer membrane is permeable) of 5 nM. See FIG. 3F. G1118 has the sequence ClAc-FWWLWDDVSWWMeFVCNH2 (SEQ ID NO: 1) or ClAc-FWWLWDDVSWWMeFVCGKKNH2 (SEQ ID NO: 2). In some embodiments, the macrocycle is G1365, which is an 8-mer macrocycle. See FIGS. 3E, 3G-L, and 4J-N. G1365 has the sequence ClAc-FVYBphMeFRVCNH2 (SEQ ID NO: 3). G1365 was identified as binding to imp E. coli MsbA, for example, with a biochemical IC₅₀ of 300 nM, and which has an affinity for the E. coli MsbA that is at least 10-fold tighter than that for the MsbA from Acinetobacter baumannii (A. bau.), when tested in imp strains with permeable outer membranes. See FIGS. 3G-L and Table 5 below.

Macrocyclic peptide activators tested include the 14-mer G1118 (SEQ ID NO: 1 or 2), as well as macrocyclic peptides G1119 (ClAc-FWWLWSDMeGDWWMeFVC-NH2; SEQ ID NO: 10) and G1122 (ClAc-FRYLWMeAWGLVWDNC-NH2; SEQ ID NO: 11), and the 8-mer G1365 (SEQ ID NO: 3).

In some embodiments, a molecule identified in a screen herein binds to the ABC transporter with a K_{D} of 20 μM or less. In some embodiments, the molecule binds to the ABC transporter with a K_{D} of 10 μM or less. In some embodiments, the molecule binds to the ABC transporter with a K_{D} of 20 nM or less. In some embodiments, the molecule binds to the ABC transporter with a K_{D} of 500 nM or less. In some embodiments, the molecule binds to the ABC transporter with a K_{D} of 1 nM or less. In some embodiments, the molecule binds to the ABC transporter with a K_{D} of 1 to 20 μM. In some embodiments, the molecule binds to the ABC transporter with a K_{D} of 10 to 20 μM. In some embodiments, the molecule binds to the ABC transporter with a K_{D} of 1 nM to 20 μM. In some embodiments, the molecule binds to the ABC transporter with a K_{D} of 1 nM to 500 nM. In some cases, the identified molecules do not bind to the chimeric ABC transporter used in the screening methods, but do bind to the parental ABC transporter on which the chimera is based. In some cases, the identified molecules bind to the parental ABC transporter with an affinity at least 10 fold tighter than to the chimeric ABC transporter used in the screen. In some cases, the identified molecules bind to the parental ABC transporter with an affinity at least 100 fold tighter than to the chimeric ABC transporter used in the screen. In some cases, the identified molecules bind to the parental ABC transporter with an affinity at least 1000 fold tighter than to the chimeric ABC transporter used in the screen. Binding affinity may be determined by methods known in the art.

In some embodiments, a molecule that is identified as binding to a particular ABC transporter can be used as a positive control or as a competitor in assays used for screening other test molecules. For example, in some methods, a molecule is identified as binding to the periplasmic, extracellular, or luminal face or cleft of an ABC transporter by determining that it competes with a molecule already known to bind to the periplasmic, extracellular, or luminal face or cleft of the same ABC transporter. For example, in the case of E. coli MsbA, the macrocycles G1118 and G1365, as well as G1119 and G1122 can be used as competitive agents or positive controls in assays to look for additional binders. In some such embodiments, screening may identify a molecule that competes with G1118, G1119, and/or G1122 for binding to the periplasmic face of E. coli MsbA when it is trapped in an outward-facing conformation, such as by treatment with Mg²⁺, ATP, and vanadate. In some embodiments, the molecule inhibits binding of G1118, G1119, and/or G1122 to the ABC transporter by at least 50, 60, 70, 80, 90 or 100% in a competition assay. In some such embodiments, screening may identify a molecule that competes with G1365 for binding to the periplasmic face of E. coli MsbA when it is trapped in an outward-facing conformation, such as by treatment with Mg²⁺, ATP, and vanadate. In some embodiments, the molecule inhibits binding of G1365 to the ABC transporter by at least 50, 60, 70, 80, 90 or 100% in a competition assay.

In some embodiments, a 14-mer macrocyclic peptide may comprise a sequence as follows: ClacF-X1-X2-L-X3-X4-D-X5-X6-X7-X8-MeF-V-C (SEQ ID NO: 17), wherein: X1 is W, V, or Y; X2 is W or Y; X3 is W or Y; X4 is S, D, V, or H; X5 is N, wherein Nis chosen from any natural amino acid other than C, or a non-natural amino acid chosen from Bph ((S)-3-([1,1′-biphenyl]-4-yl)-2-aminopropanoic acid), Dopa (L-3,4-dihydroxyphenylalanine), MeF (N-methyl-L-phenylalanine), and MeG (N-methyl-L-glycine); X6 is Y, K, A, S, D, R, or V; X7 is W, Y, or Bph; and X8 is W or Y; optionally wherein the peptide further comprises a G residue following the C residue at the C-terminal end, and wherein ClacF is N-chloroacetyl L-phenylalanine, Bph is (S)-3-([1,1′-biphenyl]-4-yl)-2-aminopropanoic acid, Dopa is L-3,4-dihydroxyphenylalanine, MeF is N-methyl-L-phenylalanine, and MeG is N-methyl-L-glycine. In some cases, X1 is W or Y. In some cases, X1 is W. In some cases, X2 is W. In some cases, X3 is W. In some cases, X4 is S, D, V, or H. In some cases, X4 is D. In some cases, X5 is V, D, H, G, or Y. In some cases, X5 is V or H. In some cases, X5 is V. In some cases, X6 is D or S. In some cases, X6 is S. In some cases, X7 is W. In some cases, X8 is W. In some of the above embodiments, the macrocycle cyclizes due to a thioether linkage between the N-terminal chloroacetyl group of ClacF and the sulfhydryl group of the C residue.

In some embodiments, an 8-mer macrocyclic peptide may comprise a sequence as follows: ClacF-X1-Y-Bph-MeF-X2-V-C (SEQ ID NO: 19), wherein: X1 is V, S, Y, W, Dopa, L, V, A, R, K, or D; and X2 is R, V, Dopa, or Y; wherein ClacF is N-chloroacetyl L-phenylalanine, Bph is (S)-3-([1,1′-biphenyl]-4-yl)-2-aminopropanoic acid, Dopa is L-3,4-dihydroxyphenylalanine, and MeF is N-methyl-L-phenylalanine. In some cases, X1 is S, Y, L, V, A, R, K, or D. In some cases, X1 is V or Y. In some cases, X1 is V. In some cases, X2 is R or Y. In some cases, X2 is R. In some embodiments, the macrocycle cyclizes due to a thioether linkage between the N-terminal chloroacetyl group of ClacF and the sulfhydryl group of the C residue.

In other cases, the macrocycle has the sequence of G1118, G1119, or G1122 (SEQ ID Nos: 1 or 2 (for G1118), 10 (G1119), or 11 (G1122)). In yet other cases, the macrocycle has the sequence of G1365 (SEQ ID NO: 3).

In some embodiments, the peptide or macrocycle is conjugated to another molecule, such as an antibiotic or antimicrobial, optionally wherein the conjugation is at the C-terminal amino acid residue of the sequence. In some such cases, the antibiotic or antimicrobial is a polymyxin, such as polymyxin B or polymyxin E.

### V. Molecular Complexes

In some embodiments, the disclosure comprises a molecular complex comprising an ABC transporter as described herein bound to a molecule, such as a peptide, small molecule, antibody, or binding fragment of a peptide, small molecule, or antibody. In some embodiments, the invention comprises a molecular complex comprising an ABC transporter and a macrocycle, which in some embodiments is a 6-14-mer, 6-10-mer, 6-8-mer, or 8-10-mer macrocycle. In some embodiments, the molecule binds to the ABC transporter with a K_{D} of 20 μM or less, 10 μM or less, 20 nM or less, 500 nM or less, 1 nM or less, 1 to 20 μM, 10 to 20 μM, 1 nM to 20 μM, and/or 1 nM to 500 nM. In some embodiments, the disclosure comprises a molecular complex comprising a chimeric ABC transporter as described herein bound to a molecule, such as a peptide, small molecule, antibody, or binding fragment of a peptide, small molecule, or antibody. In some embodiments, the invention comprises a molecular complex comprising a chimeric ABC transporter and a macrocycle, which in some embodiments is an 8-10-mer macrocycle.

In some embodiments, the molecule is a peptide. In some embodiments, the peptide is a 6-14 mer peptide, such as a 6-12-mer, a 6-10-mer, a 6-8-mer, an 8-12-mer, an 8-10-mer, or the like. In some embodiments, the peptide is a 14-mer. See Table 3 and FIG. 3F. In some embodiments, the peptide is an 6-10 mer. In some embodiments, the peptide is an 8-10 mer. In some embodiments, the peptide is an 6-8 mer. In some embodiment, the peptide is an 8-mer. See FIGS. 3E and 3G-L, and Table 5. In some embodiments, the peptide is a 3-40-mer, a 3-20-mer, a 4-16-mer, a 4-14-mer, or a 6-14-mer, such as a 3-mer, 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, or 40-mer.

In some embodiments, the peptide is a macrocycle. In some embodiments, the macrocycle is a 6-14 mer macrocycle, such as a 6-12-mer, a 6-10-mer, a 6-8-mer, an 8-12-mer, an 8-10-mer, or the like. In some embodiments, the macrocycle is a 14-mer macrocycle. See Table 3 and FIG. 3F. In some embodiments, the macrocycle is an 6-10 mer macrocycle. In some embodiments, the macrocycle is an 8-10 mer macrocycle. In some embodiments, the macrocycle is an 6-8 mer macrocycle. In some embodiments, the macrocycle is an 8-mer macrocycle. See FIGS. 3E and 3G-L, and Table 5. In some embodiments, the macrocycle is a 3-40-mer, a 3-20-mer, a 4-16-mer, a 4-14-mer, or a 6-14-mer, such as a 3-mer, 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, or 40-mer macrocycle. In some embodiments, the macrocycle has at least one lipophilic side-chain and at least one positively charged side-chain.

In some embodiments, the molecule in the complex is a small molecule. In some embodiments, the molecule is an antibody, which may include not only full length antibodies of any of IgG, IgM, IgA, IgD, and IgE, but also an antigen binding fragment of an antibody, such as an Fv, Fab′, (Fab′)2, scFv, or the like, a nanobody, single-chain antibody, bispecific or multispecific antibody.

In some embodiments, the molecule is a binding fragment of a peptide, a binding fragment of a small molecule, or a binding fragment of an antibody (e.g., an antigen binding fragment).

In some embodiments, the molecule in the complex is G1118, G1365, G1119, or G1122. In some embodiments, the molecule in the complex competes with macrocycle G1118, G1119, and/or G1122 for binding to the periplasmic face of an ABC transporter such as E. coli MsbA, for example, when the ABC transporter is trapped in an outward-facing conformation by treatment with Mg²⁺, ATP, and vanadate. In some embodiments, the molecule inhibits binding of G1118, G1119, and/or G1122 to the ABC transporter by at least 50, 60, 70, 80, 90 or 100% in a competition assay. In some embodiments, the molecule competes with macrocycle G1365 for binding to the periplasmic face of an ABC transporter such as E. coli MsbA, for example, when the ABC transporter is trapped in an outward-facing conformation by treatment with Mg²⁺, ATP, and vanadate. In some embodiments, the molecule inhibits binding of G1365 to the ABC transporter by at least 50, 60, 70, 80, 90 or 100% in a competition assay.

### VI. Uses

In some embodiments, the ABC transporter is a bacterial ABC transporter. For example, in some cases, the bacterial ABC transporter is a potential target for antibiotics, for instance, due to a relatively low sequence homology to mammalian ABC transporters. In some cases, the bacterial ABC transporter is an MsbA transporter from a bacterial species or pathogen. In some cases, the screening methods herein may be used to identify molecules that inhibit a bacterial ABC transporter. Such molecules may have antibiotic activity. For example, the macrocycles G1118 and G1365 described herein were each found to inhibit LPS transport activity of E. coli MsbA and also to inhibit cell growth. Thus, the present disclosure also encompasses the use of molecules identified in the screens herein against a bacterial ABC transporter in treating an infection in a subject, such as a bacterial infection.

### VII. Kits

The present disclosure also includes kits comprising reagents associated with screening methods herein. In some cases, kits comprise chimeric ABC transporters. In some cases, kits comprise reagents used in screening methods herein, either with or without particular chimeric ABC transporters. In some cases, kits comprise parental (non-chimeric) ABC transporters.

In some embodiments, kits herein may comprise parental or chimeric ABC transporters attached to a matrix. In some embodiments, kits herein may comprise ABC transporters attached to matrix particles such as beads. Such beads can have any shape, such as flakes or chips, spheres, pellets, etc. In some embodiments, such beads are streptavidin-coated beads, avidin-coated beads, or deglycosylated-avidin-coated beads. In some embodiments, such beads are magnetic beads. ABC transporters may or may not be pre-attached to a matrix. In some embodiments, reagents are included to facilitate attachment of ABC transporters to beads or to a matrix, such as through biotin-streptavidin or a similar system.

In some embodiments, kits may comprise reagents associated with screening methods herein. In some embodiments, kits may include some or all of the necessary reagents for determining whether a test molecule binds to the periplasmic, extracellular, and/or luminal face of an ABC transporter. Kits may comprise, for example, one or more detergents for solubilizing ABC transporters and/or chimeric ABC transporters. Exemplary detergents or related molecules or systems for solubilizing ABC transporters and/or chimeric ABC transporters include lauryl maltose neopentyl glycol (LMNG), and in some embodiments, 0.02% LMNG, as well as dodecyl-ß-D-maltoside (DDM), brij-35, glycol-diosgenin, digitonin, amphiphols such as amphiphol A8-35, and lipid nanodiscs. Kits may comprise, for example, ATP, Mg2+, vanadate, and/or sodium orthovanadate, or other reagents that trap ABC transporters in an outward-facing conformation. Kits may comprise, for example, one or more wash buffers. In some embodiments, wash buffer may comprise Tris, MgCl₂, LMNG, ATP, DTT, and/or sodium orthovanadate. In some embodiments, kits may comprise one or more elution buffers. In some embodiments, kits may comprise reagents for quantitative PCR. In some embodiments, kits may comprise reagents to running ATPase assays on ABC transporters in the presence or absence of a test molecule.

In some embodiments, kits may comprise test molecules or libraries of test molecules, such as peptides, small molecules, and/or antibodies. In some embodiments, peptides in the kit may be macrocycles. In some embodiments, the kit may comprise test molecules that are a binding fragment of a peptide, small molecule, or antibody. Kits may also include control molecules, such as positive controls known to bind to the periplasmic, extracellular, and/or luminal face of a particular ABC transporter, or negative controls that do not bind at that location, or that bind to a chimeric ABC transporter but not to its parental ABC transporter.

Kits may comprise, for example, detection reagents for detecting binding. Kits may also comprise control molecules and reagents to be used with control molecules.

In some embodiments, kits may also comprise directions for use.

### EXAMPLES

The following are examples of methods and compositions of the disclosure. It is understood that these Examples are not meant to limit the disclosure, but only to exemplify it, and that various other embodiments may be practiced, given the general description provided above.

### Introduction

MsbA is an essential ATP-binding cassette (ABC) transporter in Gram-negative bacteria that is responsible for flipping lipopolysaccharide (LPS) across the inner membrane (IM) for subsequent transport to the cell surface. Structural studies have defined an alternating access mechanism for LPS transport by MsbA, but how substrate selectivity is achieved was unknown. Prior structures of apo MsbA revealed an inward-facing conformation with LPS bound in a central vestibule, shielded from the bulk phospholipid bilayer. In this encapsulated location, the LPS is coordinated by a ring of conserved basic residues with all acyl chains enclosed in a hydrophobic cavity.

An unbiased high-throughput screen was performed with purified WT EcMsbA. By monitoring MsbA ATPase activity, quinoline and benzophenone classes of inhibitors were found to trap the inward-facing confirmation by targeting membrane-exposed binding sites. While these efforts validated MsbA as a potential antibacterial target, neither small molecule series could be progressed due to their high lipophilicity and poor physiochemical properties. Notably, all small molecule modulators of ABC transporters that have been structurally characterized to date also bind to inward-facing states, raising the possibility that traditional drug discovery approaches are inherently biased towards these inward-facing states. Thus, whether any state of the transport cycle of MsbA is permissible to drug discovery was a fundamental open question.

To overcome the challenges associated with targeting a hydrophobic, membrane-embedded binding site in MsbA, a new inhibitor discovery strategy was devisedA solvent-accessible region on the periplasmic face of MsbA was targeted in order to discover inhibitors with improved physiochemical properties. Notably, a key aspect of the MsbA transport cycle is the pronounced shift from inward- to outward-facing states, which exposes a large solvent accessible cleft to the periplasm (FIG. 6, Panel 1). Under biochemical conditions, MsbA can be trapped in an outward-facing conformation that stabilizes this periplasmic cleft for potential inhibitor discovery. Id and FIG. 3A. To bias selections towards the solvent-exposed periplasmic cleft, a counter-selection strategy was employed using MsbA chimeras in which the periplasmic face of the transmembrane segments was replaced en masse with sequences from distantly-related ABC transporters (FIGS. 3A and 9A-1B, and Example 7). After multiple rounds of screening with enrichment, G1118 and G1365 were identified as two macrocycle compounds that bind MsbA. Both G1118 and G1365 were selective and state-dependent in their binding of MsbA, and demonstrated potent biochemical and phenotypic activity. High resolution structures of these macrocycles in complex with MsbA define the molecular basis for their state-dependent inhibition and represent the first known antagonists that target the outward-facing cleft in an ABC transporter. Thus, our strategy presents a template for the development of selective modulators of MsbA and potentially for other challenging drug targets.

### Example 1: Materials and Methods for the Preparation and Expression of Chimeric MsbA Transporter Proteins

a. Design of Chimeric Proteins

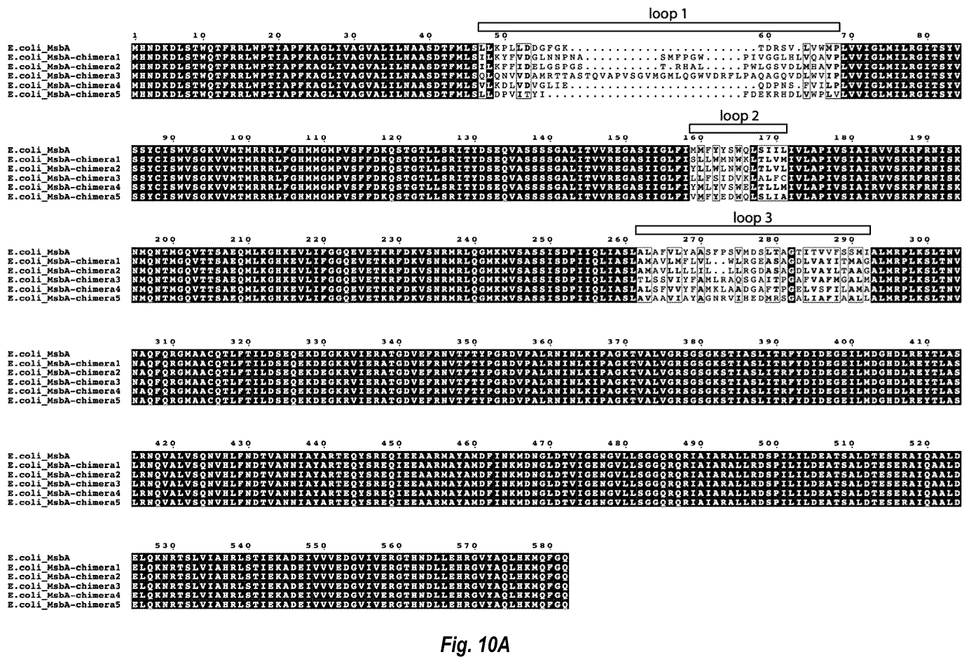

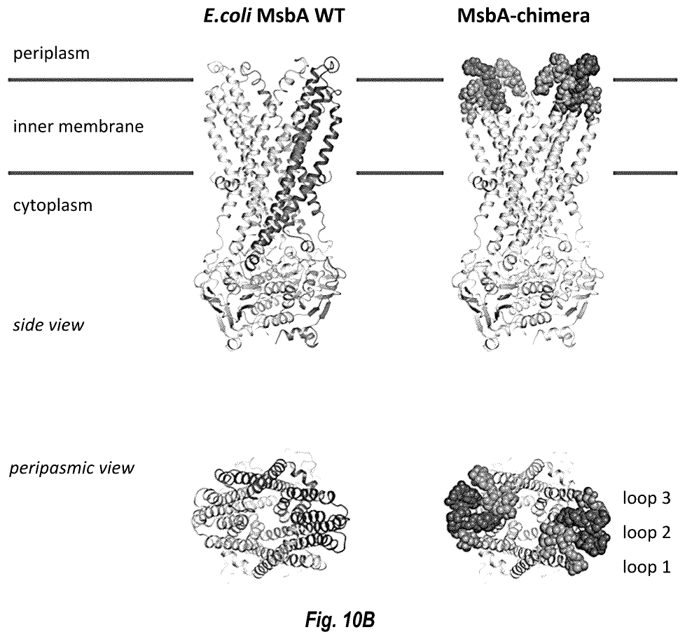

The wild-type MsbA transporter sequence from Escherichia coli (E. coli) MsbA (EcMsbA; UniProtKB: P60752) was used in a BLAST search to identify homologous proteins in other Gram-negative bacterial species. Through reiterative rounds of BLAST searching, candidate MsbA transporters with overall sequence identity to FcMsbA of ˜40%, including within the periplasmic loop regions, were selected for further consideration. Multi-sequence alignments and structural homology models (SWISSPROT) of the putative MsbA homologs were then manually analyzed to select candidates for subsequent chimeric construct engineering. Based these analyses, the putative MsbA transporters from the Gram-negative strains Pseudomonas psychrotolerans (PpMsbA), Candidatus accumulibacter (CaMsbA), Janthinobacterium agaricidamnosum (JaMsbA), Thiomicrospira cyclica (TcMsbA), and Magnetospira strain-QH-2 (MqMsbA) were selected for chimerization to EcMsbA. To generate the chimeras, the corresponding sequences of periplasmic loop 1 (L1, EcMsbA residues Leu47-Pro68), periplasmic loop 3 (L3, EcMsbA residues Met159-Leu171) and periplasmic loop 5 (L5, EcMsbA residues Ala262-Ile292) were simultaneously replaced with the equivalent regions of the putative MsbA homologues, generating the chimeric constructs (FIG. 10A).

The five chimeric constructs were synthesized (Genscript) and cloned into a pET-52b expression vector (EMD Millipore) modified for restriction-independent cloning. The chimeras were then overexpressed by autoinduction fermentation at 17° C. for 64 hours, using E. coli host Rosetta 2 (DE3; EMD Millipore). Cells were harvested and the chimeras purified as previously described for wild-type Escherichia coli (E. coli) MsbA (EcMsbA; Ho et al., Nature 557(7704): 196-201 (2018)). Chimeras generated using periplasmic sequences from the putative CaMsbA (MsbA^{Chim2}), TcMsbA (MsbA^{Chim4}), and MqMsbA (MsbA^{Chim5}) could be readily expressed and purified from E. coli, with recovery yields similar to WT MsbA. The final buffer for the purified MsbA chimeric proteins was 20 mM Tris pH 8.0, 100 mM NaCl and 0.005% lauryl maltose neopentyl glycol (LMNG; wt/v). Protein aliquots were flash frozen and stored at −80° C.

b. Protein Expression and Purification

The wild-type (WT) MsbA transporter from Escherichia coli (E. coli, EcMsbA), as well as the periplasmic chimeras, were expressed and purified as described previously (Ho et al., 2018), using n-Dodecyl-α-D-Maltoside (αDDM, for cryo-EM structural studies and some biochemical assays, as indicated; Anatrace), lauryl maltose neopentyl glycol (LMNG, for biochemical assays; Anatrace), or 3α-hydroxy-7α,12α-di-((O-β-D-maltosyl)-2-hydroxyethoxy)-cholane (FA3, for crystallography studies) as the solubilizing detergents. The WT MsbA proteins from Enterobacter cloacae (E. cloacae; EnMsbA), Klebsiella pneumoniae (K. pneumoniae; KpMsbA), and Pseudomonas aeruginosa (P. aeruginosa; PaMsbA) were similarly expressed and purified, using LMNG as the solubilizing detergent. The final buffer for the purified wild-type MsbA proteins was 20 mM Tris pH 8.0, 100 mM NaCl and 0.03% (wt/v) DDM or 0.02% LMNG (wt/v). Protein aliquots were flash frozen and stored at −80° C.

In some cases, a recognition motif for E. coli BirA biotin ligase (GLNDIFEAQKIEWHE; SEQ ID NO: 18) was included between the amino terminal FLAG affinity tag sequence, to allow for efficient site-specific biotinylation of the purified MsbA (Avidity). Evaluation of the ATPase activity and pharmacology of the chimeras revealed that these large sequence changes could be tolerated without significant loss of biochemical activity or apparent structural alteration of the known antagonist receptor site. In particular, MsbA^{Chim5} replaced the periplasmic face of EcMsbA with sequences from the putative MsbA homologue from Magnetospira spirillum strain-QH-2, yet retained sensitivity to both the quinoline and benzophenone classes of previously identified small molecule MsbA inhibitors.

For biochemical evaluation of the E. coli MsbA point mutants, WT EcMsbA and the Tyr87Ala/Trp91Ala (FcMsbA Y87A/W91A) and Lys95Ala/Arg238Ala (EcMsbA K95A/R238A) mutants were synthesized (Genscript) and cloned into a modified p15A plasmid (pLMG18) (Storek et al., 2018), downstream of the Ptac promoter.

MsbA was overexpressed using E. coli host BL21 and induction was performed with 1 mM IPTG at 37° C. for 3 hours.

Cells expressing MsbA were harvested and resuspended in 50 mM Tris, pH 8.0, 500 mM NaCl (Buffer A) supplemented with cOmplete™ Protease Inhibitors (Roche), 1 mM phenylmethylsulfonyl fluoride (PMSF) and 2 units/mL of Benzonase nuclease (Sigma-Aldrich). Following cell lysis by microfluidization, LMNG was added to 1% (wt/v) and protein solubilization was carried out with gentle agitation at 4° C. overnight. After centrifugation at 185,000×g for 1 hour, clarified supernatant was mixed gently with anti-FLAG M2-agarose resin (Sigma) pre-equilibrated with Buffer B (Buffer A supplemented with 0.005% LMNG (wt/v)) for 1-2 hours at 4° C. FLAG resin was collected by gravity flow, washed twice with ten column volumes of Buffer B, and eluted twice with three column volumes of Buffer B supplemented with 0.2 mg/mL FLAG peptide. Each wild-type or mutant MsbA protein was passed over a Superdex® 200 column (GE Healthcare) or a Superose 6 Increase column (Cytiva) in 20 mM Tris pH 8.0, 100 mM NaCl and 0.005% LMNG (wt/v). The peak fractions containing MsbA were pooled and concentrated to 5-10 mg/mL using Vivaspin® centrifugal devices (50K molecular weight cutoff). Protein aliquots were flash frozen and stored at −80° C.

c. Reconstitution Into Amphipols

Where the use of amphipol-incorporated MsbA is indicated, the purified MsbA in detergent was adjusted to 1 mg/mL and reconstituted into A8-35 amphipols (Zoonens & Popot, 2014) (Anatrace) as described previously (Ho et al., 2018). The final buffer for the purified, amphipol-incorporated MsbA proteins was 20 mM Tris pH 8.0 and 100 mM NaCl. Protein aliquots were flash frozen and stored at −80° C.

d. Sequence Conservation Analysis

MsbA sequences from the enterobacteriaceae family of Gram-negative bacteria (taxid: 543) were identified using a BLAST search against the refseq_select database using EcMsbA as a query sequence (Uniprot ID: P60752). A total of 118 sequences from this search annotated as MsbA homologs were aligned using Constraint-based Multiple Alignment Tool (COBALT) (Papadopoulos & Agarwala, 2007). The multisequence alignment was loaded into ChimeraX and the sequence conservation was determined using the sum of pairs method in AL2CO (Pei & Grishin, 2001).

### Example 2: Screening Materials and Methods for Identifying MsbA-Binding Macrocycles

a. Macrocyclic Peptide Library Design

Peptide macrocycles were tested to identify molecules binding to the periplasmic face of EcMsbA, using a counterselection with the chimeric MsbA proteins (FIG. 3A). A thioether-macrocyclic peptide library was constructed by using N-chloroacetyl L-phenylalanine (ClAc-F) as an initiator in a genetically reprogrammed in vitro translation system (Kashiwagi et al., 2013). The genetic code was designed with the addition of N-methyl-L-phenylalanine (MeF) and (S)-3-([1,1′-biphenyl]-4-yl)-2-aminopropanoic acid (Bph), in addition to all 20 natural amino acids except cysteine. After in vitro translation, a thioether bond formed spontaneously between the N-terminal ClAc group of the initiator L-phenylalanine residue and the sulfhydryl group of a downstream cysteine residue to generate the macrocyclic peptides.

b. Selection of Periplasmic MsbA-Binding Molecules

Affinity selection of macrocyclic peptides binding to MsbA was performed using site-specifically biotinylated EcMsbA solubilized in 0.02% LMNG and trapped in an outward-facing conformation by inclusion of 50 μM ATP, 10 mM Mg²⁺ and 200 μM sodium orthovanadate throughout the selections. Briefly, 10 μM mRNA library was hybridized with a peptide-linker (11 μM) at room temperature (RT) for 3 minutes. The mRNA library was translated at 37° C. for 30 minutes in the reprogrammed in vitro translation system to generate the peptide-mRNA fusion library (Goto et al., 2011; Kawakami et al., 2013). Each reaction contained 2 μM mRNA-peptide-linker conjugate, 12.5 μM initiator tRNA (tRNAfMet aminoacylated with ClAc-L-Phe), and 25 μM of each elongator tRNA aminoacylated with the specified non-canonical/canonical amino acids. In the first round of selections, translation was performed at 20 μL scale. After the translation, the reaction was quenched with 17 mM EDTA. The product was subsequently reverse-transcribed using RNase H minus reverse transcriptase (Promega) at 42° C. for 30 minutes and buffer was exchanged for vanadate buffer: 50 mM Tris pH 7.5, 10 mM MgCl₂, 0.02% LMNG (wt/v), 50 uM ATP, 1 mM DTT and 200 uM sodium orthovanadate.

For affinity selection, the peptide-mRNA/cDNA solution was incubated with 250 nM biotinylated FcMsbA and 500 nM EcMsbAChim5 as sink for 60 minutes at 4° C. The streptavidin-coated beads (Dynabeads™ M-280 Streptavidin, ThermoFisher Scientific) were further added and incubated for 10 minutes to isolate macrocycles binding to the periplasmic face of EcMsbA. The beads were washed three times with cold vanadate buffer, the cDNA was eluted from the beads by heating for 5 minutes at 95° C., and fractional recovery from the affinity selection step was assessed by quantitative PCR using Sybr Green I on a LightCycler™ thermal cycler (Roche). After six rounds of affinity maturation, two additional rounds of off-rate selections were performed by increasing the wash stringency before elution to identify high affinity binders. Sequencing of the final enriched cDNA was carried out using a MiSeq next generation sequencer (Illumina).

c. Mutational Scan of G1118 and G1365 Macrocycles

For the mutational scan of the G1118 (Table 4 below) and G1365 (Table 6 below) macrocycles, site saturation DNA libraries were constructed based on these parental sequences by pooling multiple DNA templates, each containing a single ‘NNU’ degenerate codon at different positions so that, after translation, every single amino acid change to the parental sequence was sampled. The genetic code was designed with the addition of Pro, Lys, Ala, Dopa (L-3,4-dihydroxyphenylalanine), and MeG (N-Methyl-L-glycine), in addition to the amino acids used in the parental peptides. After in vitro translation, one round of affinity selections was performed independently using the two macrocyclic libraries as described above. The input and recovered output DNA pools were subjected to deep sequencing by NGS. The enrichment factor for each single mutant was calculated as follows: the NGS frequency in the output pool divided by the one in the input pool, normalized by the parent value.

### Example 3: Materials and Methods for Characterization of MsbA Enzyme Variants and Bacterial Strains

a. Plasmids
The following plasmids (Table 1) were used in E. coli phenotypic studies.

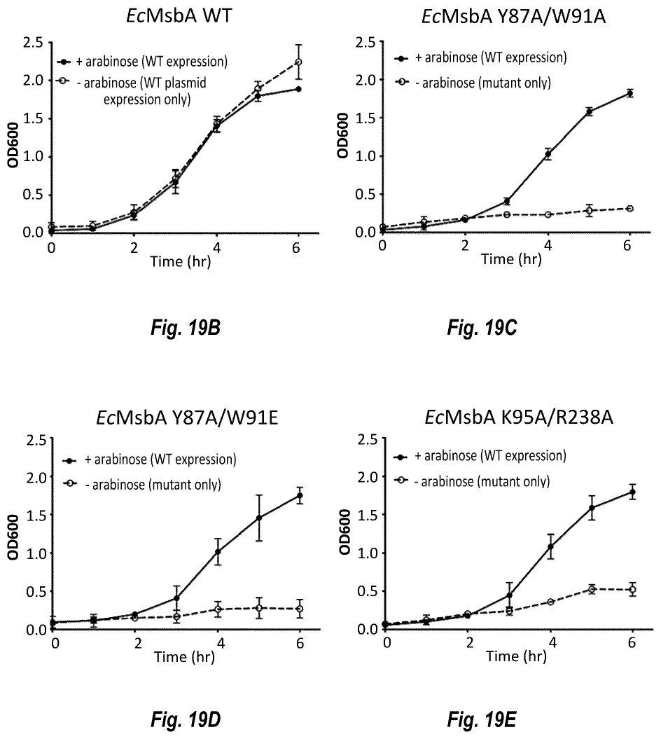

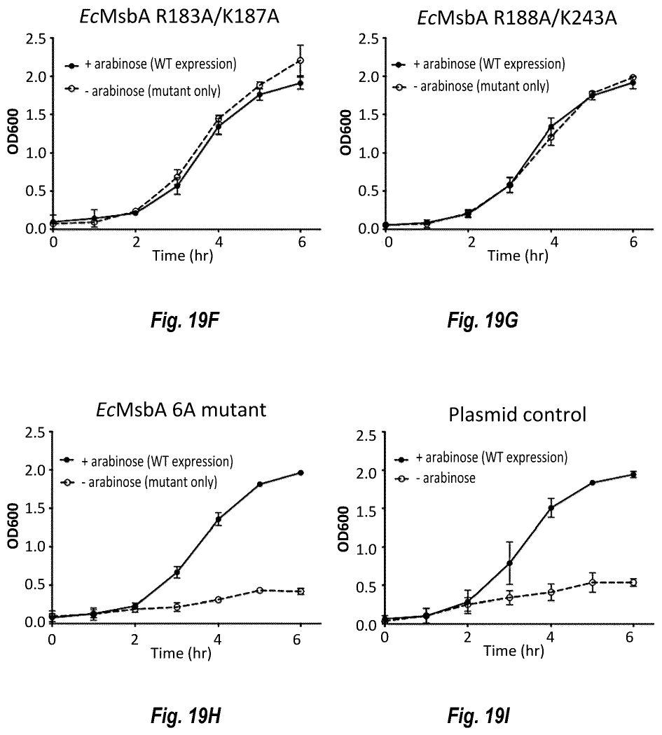

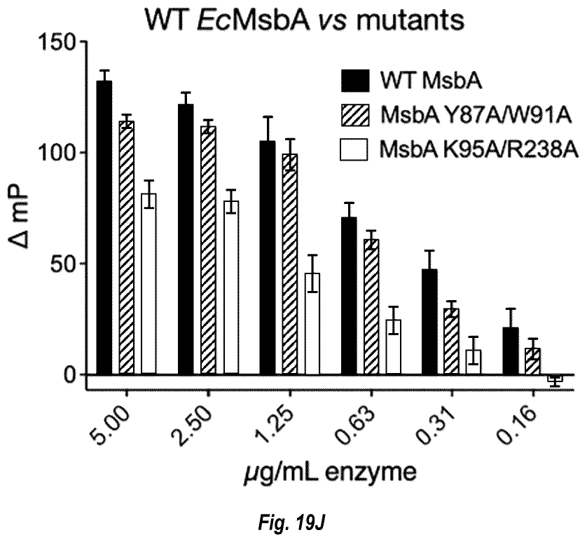

TABLE 1
Plasmids.

| Plasmid name | Description |
|---|---|
| pLMG18 | Low-copy IPTG-inducible expression vector |
| | with chloramphenicol resistance marker. |
| | WT MsbA and mutants display constitutive |
| | ‘leaky’ expression without IPTG |
| | induction, to levels comparable to |
| | endogenous WT MsbA (FIG. 19A). (Storek |
| | et al., 2018) |
| pLMG18-EcMsbA | E. coli MsbA ORF cloned into pLMG18. |
| | (Alexander et al., 2018) |
| pLMG18-FLAG-EcMsbA | E. coli MsbA ORF with N-terminal FLAG |
| | tag cloned into pLMG18. Peripheral LPS |
| | binding site mutants are also cloned |
| | into this vector. |

b. MsbA ATPase Assay

The ATPase activity of MsbA was measured using a Transcreener ADP2 FP Assay (BellBrook Labs). To determine the IC₅₀ of MsbA inhibitors, compounds were incubated with 2× MsbA enzyme solution for 10 min and then 2× ATP solution was added to initiate the ATPase reaction. The final condition for the reaction was MsbA enzyme, 50 μM ATP in 50 mM Tris pH 7.5, 10 mM MgCl2, 1% glycerol, 0.1% bovine gamma globulin, 1 mM dithiothreitol (DTT), 0.007% Brij-35 and 0.5% dimethylsulfoxide (DMSO). The following concentrations were used for each MsbA variant: 5 nM of EcMsbA, EnMsbA, KpMsbA and 80 nM PaMsbA reconstituted in amphipol, 15 nM EcMsbA and EcMsbA-chim5 in LMNG detergent, 22 nM EcMsbA and 27 nM EcMsbA-12G7 complex in aDDM detergent. The enzyme reaction was incubated for 1 hour at room temperature, then quenched by adding the Transcreener Detection Buffer. The IC₅₀ was determined by fitting the inhibition dose-response curve with a nonlinear four-parameter inhibition model (GraphPad Prism).

c. Cellular IC₅₀ Determination

The cellular IC₅₀'s of various bacterial strains (Table 2) were determined.

TABLE 2
Bacterial Strains.

| Strain name | Description |
|---|---|
| MG1655 msbA-cKO | E. coli K12 with arabinose-inducible |
| lptD(imp4213) | conditional knockout of msbA and |
| | permeable outer membrane (Alexander |
| | et al., 2018) |
| MG1655 lptD(imp4213) | E. coli K12 with permeable outer membrane |
| | (Alexander et al., 2018) |
| MG1655 lptD(imp4213) | E. coli K12 with permeable outer membrane, |
| msbA(D252N) | resistant to G1365 |
| CFT073 | E. coli clinical isolate from a urinary |
| | tract infection (ATCC 700928) |
| CFT073 lptD(imp4213) | E. coli clinical isolate with permeable |
| | outer membrane (Ho et al., 2018) |

200 nL aliquots of eight 2-fold dilutions of each compound were dispensed in duplicate into 384-well plates using an Echo Liquid Handler (Labcyte). Strains were grown in cation-adjusted Mueller Hinton Broth with 0.002% Tween overnight for 16 hours, washed with PBS, resuspended at OD₆₀₀=1 in cation-adjusted Mueller Hinton Broth with 0.002% Tween and 10% glycerol, and frozen in 100 ml aliquots. For each experiment, aliquots were thawed on ice and diluted 1:1000 into cation-adjusted Mueller Hinton Broth with 0.002% Tween. 10 μL cells/well were added to 384-well plates containing compound. Plates were incubated for 6 hours at 37° C., then 10 μL BacTiter-Glo reagent (Promega) was added to each well and luminescence was measured. IC₅₀'s were calculated using Genedata Screener software.

d. Time Course of Growth of E. Coli Complemented With MsbA Alleles

Cultures of E. coli MG1655 msbA-cKO lptD(imp4213) carrying a pLMG18 vector expressing WT or mutant alleles of E. coli MsbA were grown overnight at 37° C. in 5 mL LB with 10 μg/ml chloramphenicol and 2% arabinose to induce expression of WT E. coli MsbA from the chromosome. Overnight cultures were pelleted, washed with 5 ml phosphate-buffered saline, and resuspended in LB at OD600=1. Each culture was then diluted 1:100 into 5 mL LB, 5 mL LB+2% arabinose. All cultures were grown on a roller drum at 37° C., and OD600 was measured hourly. Growth curves were repeated three times on different days.

e. Western Blot

Cultures of E. coli MG1655 msbA-cKO IptD(imp4213) carrying a pLMG18 vector expressing WT or mutant alleles of E. coli MsbA were grown overnight at 37° C. in 5 mL LB with 10 μg/ml chloramphenicol and 2% arabinose to induce expression of WT E. coli MsbA from the chromosome. The no-plasmid control strain was grown under the same conditions, but without chloramphenicol. Overnight cultures were diluted 1:100 in 15 mL of the same media in 50-ml conical tubes and grown with shaking for 3 hours at 37° C. These cultures were pelleted, washed with 15 mL PBS, and resuspended at OD600=1 in LB. Each culture was then diluted 1:100 in 20 mL LB for strains expressing E. coli MsbA alleles or the no-plasmid control, or 20 mL LB+1 mM IPTG for strains expressing A. baumannii MsbA alleles. 7.5 mL of each of the OD=1 cells were pelleted and lysed as the 2% arabinose samples. The diluted cultures were grown for 3 hours at 37° C., then pelleted and lysed.

To generate lysates, each sample was resuspended in 500 μl MSD Lysis buffer (150 mM NaCl, 20 mM Tris, pH 7.5, 1 mM EDTA, 1 mM EGTA, 1% Triton X-100)+15 mg/mL lysozyme and incubated 30 minutes at 37° C. Lysates were pelleted at 4° C. for 10 minutes at full speed in a microcentrifuge, and the supernatant was transferred to a clean tube. Samples were resolved in MES buffer on 20-well 4-12% Bis-Tris midi gels (BioRad), with loading normalized to OD₆₀₀ using GroEL as a loading control. Western transfer to nitrocellulose was carried out using an iBlot system (Thermo Fisher Scientific). Membranes were blocked with Odyssey TBS blocking buffer (Li-Cor, #927-50000), and detected with 1:1000 anti-FLAG M2 (Sigma, #F3165)+1:10000 anti-GroEL (Enzo, #ADI-SPS-875), followed by 1:10000 each of IRDye 800CW anti-mouse and IRDye 680RD anti-rabbit (Li-Cor, #925-32210 and #926-68071).

f. MIC Assays

Broth MICs were determined in cation-adjusted Mueller-Hinton broth with 0.002% Tween-80, as previously described (Ho et al., 2018).

g. Isolation of PD-1365 Resistant Mutant

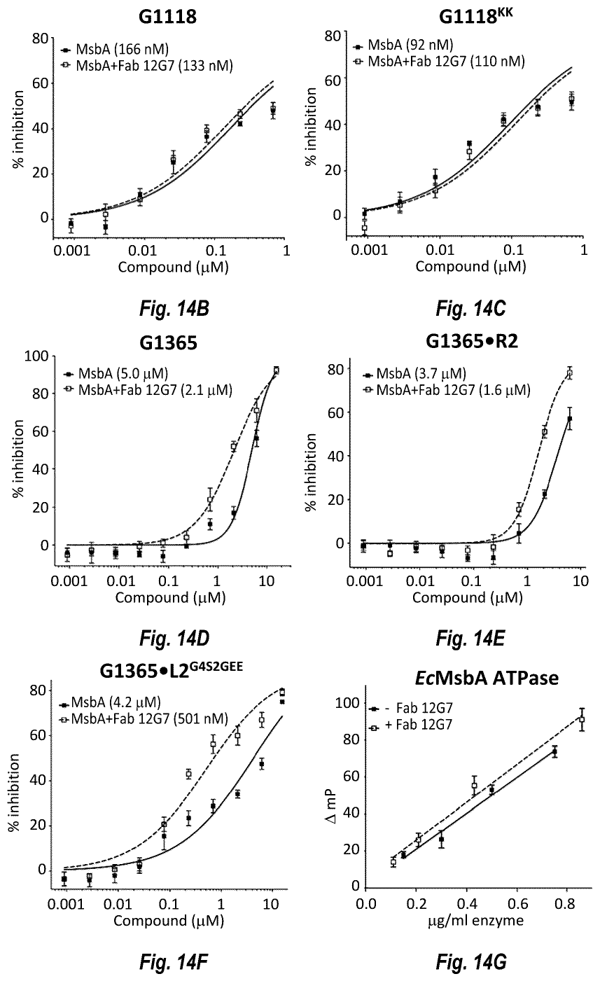

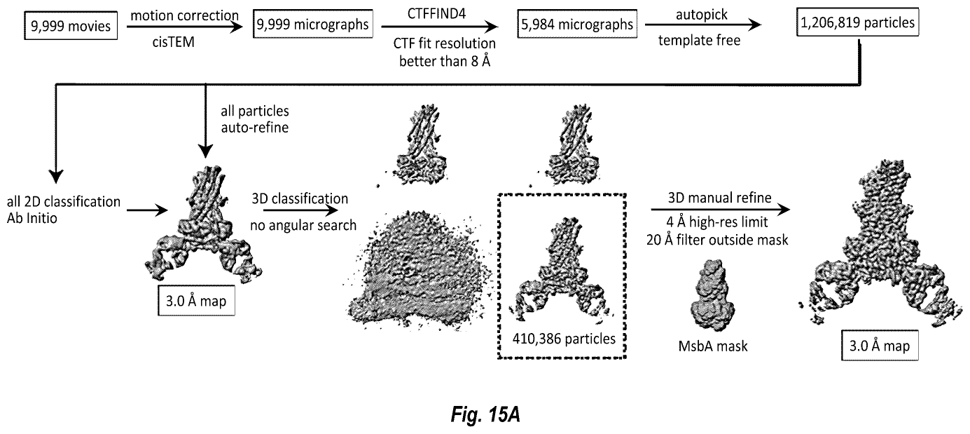

MG1655 lptD(imp4213) was inoculated at an OD₆₀₀ of 0.005 in a 96-well plate with 100 μL/well Mueller-Hinton broth+0.002% Tween-80+50 μM G1365·I2,Dopa3 (4× MIC), and grown for two days at 37° C. G1365·I2,Dopa3 is a close analog of G1365 with two amino acid substitutions (Val2Ile and Tyr3Dopa; FIG. 14A). Cultures from the two wells that displayed growth were streaked on an LB agar plate containing 50 μM G1365·I2,Dopa3; only one culture produced colonies on the selective plate. The MsbA ORF from this resistant strain was PCR amplified and sequenced, revealing a point mutation resulting in an amino acid change from aspartic acid to asparagine at residue 252. MIC assays on the parent strain and the msbA(D252N) mutant confirmed that the MIC of G1365·I2,Dopa3 increased from 12.5 μM in the parent strain to 100 μM in MG1655 lptD(imp4213) msbA(D252N).

### Example 4: Electron Microscopy Materials and Methods

Electron microscopy of E. coli CFT073 lptD(imp4213) or CFT073 lptD(imp4213) msbA-cond-ko cells rescued with pLMG18 plasmids expressing the indicated E. coli MsbA mutants was performed as described previously (Ho et al., 2018). Where the use of macrocycle or small molecule inhibitor of MsbA is indicated, an overnight culture of CFT073 lptD(imp4213) grown in LB was diluted 1:100 in LB and grown into log phase for 2.5 hours at 37° C., then pelleted, washed twice with PBS, and resuspended at OD₆₀₀=0.1 in LB. 50 μl of a 10 mM stock of the indicated inhibitor was then added to 5 mL of the resuspended CFT073 lptD(imp4213), and cultures were incubated for 3 hour at 37° C. Cells were pelleted, washed, fixed and processed for transmission electron microscopy, as described previously (Ho et al., 2018).

### Example 5: Synthesis of Macrocycle Compounds

a. Synthesis of Benzophenone (G758)

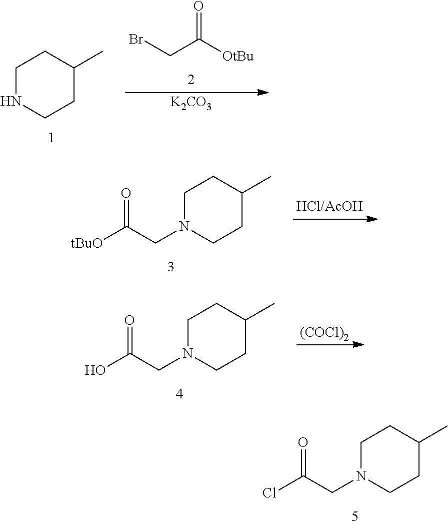

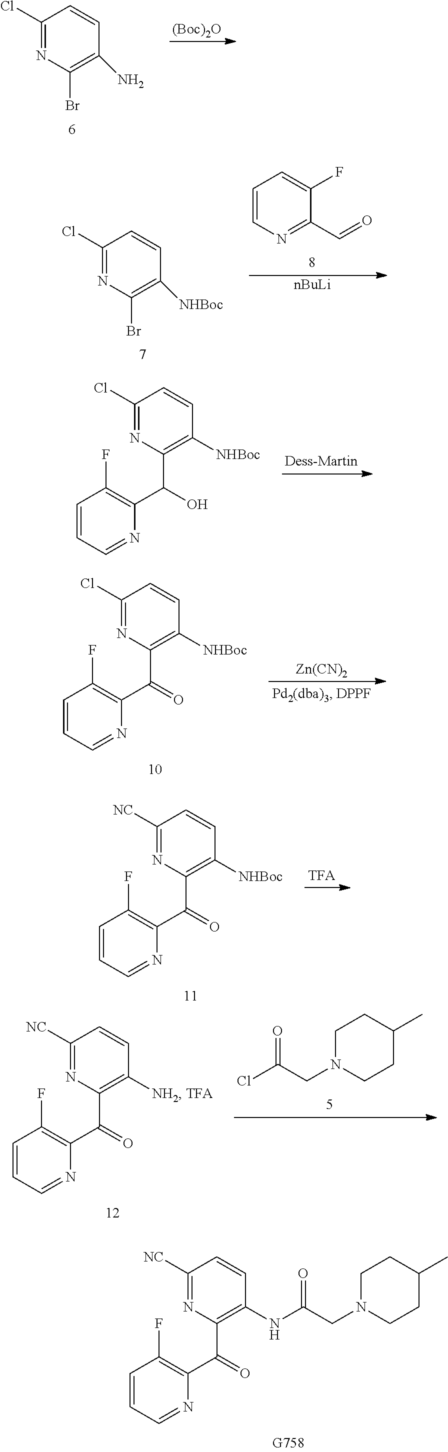

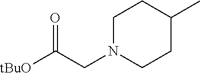

### Step 1: Tert-butyl 2-(4-methylpiperidin-1-yl)acetate

A mixture of 4-methylpiperidine (25.0 g, 252.1 mmol), potassium carbonate (104.5 g, 756.3 mmol) and tert-butyl bromoacetate (49.2 g, 252.1 mmol) in acetonitrile (400 mL) was stirred at 25° C. for 12 hours and concentrated under reduced pressure. The residue was purified by column chromatography eluting with 0% to 30% ethyl acetate in petroleum ether to afford tert-butyl 2-(4-methyl-1-piperidyl)acetate (45.0 g, 189.9 mmol, 75.3% yield) as a pale yellow solid.

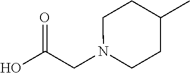

### Step 2: 2-(4-methylpiperidin-1-yl)acetic Acid

A solution of tert-butyl 2-(4-methyl-1-piperidyl)acetate (45.0 g, 211.0 mmol) and hydrochloric acid (4 N, 369.2 mL, 1476.7 mmol) in acetic acid (150 mL) was stirred at 25° C. for 14 hours and concentrated under reduced pressure to give the crude product. This crude was used directly without further purification. 1H NMR (400 MHZ, DMSO-d6): δ 4.07 p.p.m. (s, 2H), 3.42 (br. s., 2H), 3.02 (br. s., 2H), 1.77 (d, J=12.8 Hz, 2H), 1.59 (br. s., 1H), 1.51-1.36 (m, 2H), 0.92 (d, J=6.8 Hz, 3H).

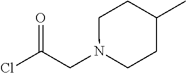

### Step 3: 2-(4-methylpiperidin-1-yl)acetyl Chloride

To a mixture of 2-(4-methyl-1-piperidyl)acetic acid (1.0 g, 6.7 mmol) in dichloromethane (20 mL) was added oxalyl chloride (1.4 mL, 15.9 mmol) and 2 drops of N,N-dimethylformamide. The resulting mixture was stirred at 20° C. for 1.5 hours and concentrated under reduced pressure to give crude 2-(4-methyl-1-piperidyl)acetyl chloride (1.0 g, 5.7 mmol, 89.5% yield). The crude was used directly for the next step without further purification.

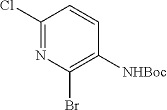 [SMILES: CC(C)(C)OC(=O)Nc1ccc(Cl)nc1Br]

### Step 4: Tert-butyl (2-bromo-6-chloropyridin-3-yl)carbamate

A solution of 2-bromo-6-chloro-pyridin-3-amine (2.5 g, 12.1 mmol), di-tert-butyldicarbonate (2.6 g, 12.1 mmol), 4-dimethylaminopyridine (294.4 mg, 2.4 mmol) and triethylamine (2438.78 mg, 24.1 mmol) in dichloromethane (20 mL) was stirred at 30° C. for 12 hours and diluted with dichloromethane (50 mL). The solution was washed with water (2×15 mL), brine (15 mL), dried over anhydrous sodium sulfate and concentrated under reduced pressure. The residue was purified by column chromatography eluting with 0% to 30% ethyl acetate in petroleum ether to afford tert-butyl N-(2-bromo-6-chloro-3-pyridyl)carbamate (1.3 g, 4.2 mmol, 35.1% yield) as a white solid.

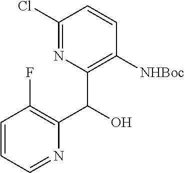 [SMILES: CC(C)(C)OC(=O)Nc1ccc(Cl)nc1C(O)c1ncccc1F]

### Step 5: Tert-butyl (6-chloro-2-((3-fluoropyridin-2-yl)(hydroxy)methyl)pyridin-3-yl)carbamate

To a solution of tert-butyl N-(2-bromo-6-chloro-3-pyridyl)carbamate (1.0 g, 3.25 mmol) in tetrahydrofuran (20 mL) was added butyl lithium (2.5 M in hexanes, 1.6 mL, 3.90 mmol) at −78° C. The mixture was stirred at −78° C. for 1 hour, then 3-fluoro-2-formylpyridine (610.1 mg, 4.88 mmol) was added. The resulting mixture was stirred at −78° C. for 4 hours and quenched by addition of saturated ammonium chloride (10 mL). The solution was diluted with ethyl acetate (50 mL) and washed with water (2×10 mL), brine (10 mL), dried over anhydrous sodium sulfate and concentrated under reduced pressure. The residue was purified by column chromatography eluting with 0% to 40% ethyl acetate in petroleum ether to afford tert-butyl N-[6-chloro-2-[(3-fluoro-2-pyridyl)-hydroxy-methyl]-3-pyridyl]carbamate (550.0 mg, 1.55 mmol, 47.8% yield) as yellow oil.

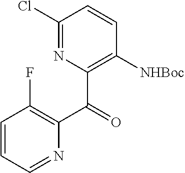 [SMILES: CC(C)(C)OC(=O)Nc1ccc(Cl)nc1C(=O)c1ncccc1F]

### Step 6: Tert-butyl (6-chloro-2-(3-fluoropicolinoyl)pyridin-3-yl)carbamate

A mixture of tert-butyl N-[6-chloro-2-[(3-fluoro-2-pyridyl)-hydroxy-methyl]-3-pyridyl]carbamate (460.0 mg, 1.30 mmol) and Dess-Martin periodinane (2.2 g, 5.20 mmol) in dichloromethane (15 mL) was stirred at 20° C. for 3 hours. The reaction mixture was diluted with dichloromethane (20 mL). washed with water (2×10 mL), brine (10 mL), dried over anhydrous sodium sulfate and concentrated under reduced pressure. The residue was purified by column chromatography eluting 0% to 30% ethyl acetate in petroleum ether to afford tert-butyl N-[6-chloro-2-(3-fluoropyridine-2-carbonyl)-3-pyridyl]carbamate (300.0 mg, 0.85 mmol, 65.6% yield) as a white solid.

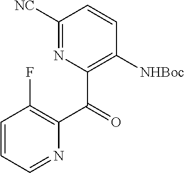 [SMILES: CC(C)(C)OC(=O)Nc1ccc(C#N)nc1C(=O)c1ncccc1F]

### Step 7: Tert-butyl (6-cyano-2-(3-fluoropicolinoyl)pyridin-3-yl)carbamate

A mixture of tert-butyl N-[6-chloro-2-(3-fluoropyridine-2-carbonyl)-3-pyridyl]carbamate (230.0 mg, 0.65 mmol), tris(dibenzylideneacetone)dipalladium (59.9 mg, 0.07 mmol), zinc cyanide (383.9 mg, 3.27 mmol) and cyclopentyl(diphenyl)phosphane iron (72.5 mg, 0.13 mmol) in N,N-dimethylacetamide (10 mL) was heated at 90° C. under microwave conditions for 2 hours and concentrated under reduced pressure. The residue was purified by column chromatography eluting 0% to 50% ethyl acetate in petroleum ether to afford tert-butyl N-[6-cyano-2-(3-fluoropyridine-2-carbonyl)-3-pyridyl]carbamate (150.0 mg, 0.44 mmol, 67.0% yield) as a white solid.

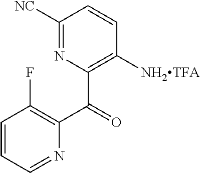

### Step 8: 5-amino-6-(3-fluoropicolinoyl)picolinonitrile Trifluoroacetate

A solution of tert-butyl N-[6-cyano-2-(3-fluoropyridine-2-carbonyl)-3-pyridyl]carbamate (120.0 mg, 0.35 mmol) and trifluoroacetic acid (4.0 mL, 51.22 mmol) in dichloromethane (16 mL) was stirred at 15° C. for 1 hour and concentrated under reduced pressure to afford crude 5-amino-6-(3-fluoropyridine-2-carbonyl)pyridine-2-carbonitrile trifluoroacetate (84.0 mg, 0.25 mmol, 71.4% yield) as yellow oil.

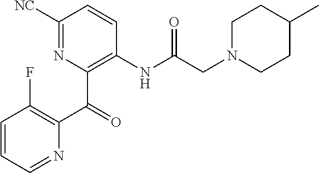 [SMILES: CC1CCN(CC(=O)Nc2ccc(C#N)nc2C(=O)c2ncccc2F)CC1]

### Step 9: N-(6-cyano-2-(3-fluoropicolinoyl)pyridin-3-yl)-2-(4-methylpiperidin-1-yl)acetamide

A mixture of 2-(4-methyl-1-piperidyl)acetyl chloride (121.8 mg, 0.69 mmol) and 5-amino-6-(3-fluoropyridine-2-carbonyl)pyridine-2-carbonitrile (84.0 mg, 0.35 mmol) in tetrahydrofuran (5 mL) was stirred at 60° C. for 2 hours and concentrated under reduced pressure. The residue was purified by reverse phase chromatography (acetonitrile 28-58/0.05% NH4OH in water) to afford N-[6-cyano-2-(3-fluoropyridine-2-carbonyl)-3-pyridyl]-2-(4-methyl-1-piperidyl)acetamide (9.40 mg, 0.02 mmol, 6.8% yield) as a white solid. 1H NMR (400 MHZ, Methanol-d4): δ 9.34 p.p.m. (d, J=8.8 Hz, 1H), 8.53 (d, J=4.8 Hz, 1H), 8.04 (d, J=4.8 Hz, 1H), 7.85-7.78 (m, 1H), 7.72-7.63 (m, 1H), 3.23 (s, 2H), 2.95-2.85 (m, 2H), 2.35-2.23 (m, 2H), 1.68-1.49 (m, 4H), 1.45-1.36 (m, 1H), 0.96 (d, J=6.4 Hz, 3H). LCMS (m/z): [M+H]+ calcd C20H20FN5O2, 381.16; found 382.1.

b. Synthesis of G1118, G1365, and Derivatives

Thioether macrocyclic peptides were synthesized using standard Fmoc solid phase peptide synthesis (SPPS). Following coupling of all amino acids, the deprotected N-terminus was chloroacetylated on-resin followed by global deprotection using a trifluoroacetic acid (TFA) deprotection cocktail. The peptides were then precipitated from the deprotection solution by adding over 10-fold excess diethyl ether. Crude peptide pellets were then dissolved and re-pelleted 3 times using diethyl ether. After the final wash, the pellet was left to dry and then the pellet was resuspended in DMSO followed by the addition of triethylamine for intramolecular cyclization via formation of a thioether bond between the thiol of the cysteine and N-terminal chloroacetyl group. Upon completion of cyclization, the reaction was quenched with AcOH and the cyclic peptide was purified using standard reverse-phase HPLC methods. The molecular masses were confirmed by single quadrupole LC/MS (LCMS-2020 systems, Shimadzu).

c. Synthesis of Other 14-mer Peptides

Further 14-mer peptides were designed according to the following formula (See, e.g., FIG. 4E for a pictoral depiction of certain variants): ClacF-X1-X2-L-X3-X4-D-X5-X6-X7-X8-MeF-V-C (SEQ ID NO: 17), wherein:
i. X1 is W, V, or Y;
ii. X2 is W or Y;
iii. X3 is W or Y;
iv. X4 is S, D, V, or H;
V. X5 is N, wherein N is chosen from any natural amino acid other than C, or a non-natural amino acid chosen from Bph ((S)-3-([1,1′-biphenyl]-4-yl)-2-aminopropanoic acid), Dopa (L-3,4-dihydroxyphenylalanine), MeF (N-methyl-L-phenylalanine), and MeG (N-methyl-L-glycine);
vi. X6 is Y, K, A, S, D, R, or V;
vii. X7 is W, Y, or Bph; and
viii. X8 is W or Y;
   optionally wherein the peptide further comprises a G residue following the C residue at the C-terminal end, and wherein ClacF is N-chloroacetyl L-phenylalanine, Bph is (S)-3-([1,1′-biphenyl]-4-yl)-2-aminopropanoic acid, Dopa is L-3,4-dihydroxyphenylalanine, MeF is N-methyl-L-phenylalanine, and MeG is N-methyl-L-glycine. Eleven different molecules according to this formula were also tested for inhibitory effects on MsbA ATPase activity, in an assay as described in Example 3 above, and found to have IC₅₀ values of 0.2 to 0.8 μM. The minimum inhibitory concentration (MIC) for inhibition of E. coli MsbA (EcMsbA) was more than 100 μM, and the EC₅₀ for inhibition of growth of cells of a UPEC imp strain was from about 2 to about 8 μM for all but one of the tested molecules (and over 100 μM for the final tested molecule). Collectively, these data indicate that macrocyclic peptides G1118 and related peptides according to the formula above are generally able to inhibit MsbA and cellular activity related to MsbA.

### Example 6: Materials and Methods Related to Protein Structure Determination

a. Protein Crystallization

Inhibitor G758 was added to MsbA-chim5 at 10 mg ml/L purified in FA3 to a final concentration of 1 mM and incubated for 1 hour on ice. Exogenous LPS was not added at any point. The G758-LPS-MsbA-chim5 complex was crystallized at 19° C. using sitting-drop vapor diffusion by mixing the complex with mother liquor (150 mM NaF, 13% PEG 3350, 100 mM HEPES, pH 7.0) at a 1:1 (v/v) ratio. After reaching full size in one week, to improve diffraction, the crystals were dehydrated by first soaking in 10% ethylene glycol, 15% PEG 3350, 100 mM HEPES, pH 7.0 for 30 minutes then in 20% ethylene glycol, 20% PEG 3350, 100 mM HEPES, pH 7.0 for another 30 minutes before flash freezing in liquid nitrogen.

b. Structure Determination, Refinement and Analysis

Diffraction data were collected at 100 K using beamline 17ID of the Advanced Light Source. X-ray diffraction data were integrated and scaled using autoPROC (Vonrhein et al., 2011) including anisotropy correction performed using STARANISO (Tickle, et al. (2020) Global Phasing Ltd; staraniso.globalphasing.org/cgi-bin/staraniso.cgi). The structure was determined by molecular replacement using PHENIX (Adams et al., 2010) using one MsbA subunit from PDB 6BPL as the search model. Manual adjustments to the initial solution were performed through rigid body movement in Coot (Emsley et al., 2010). Reiterative rounds of refinement and model building in Coot were guided by inspection of omit maps. Combined with the use of omit maps at early stages in refinement, the chimera 5 sequence was replaced with the WT E. coli MsbA sequence in the model. Strict geometry and secondary structure restraints were applied throughout refinement to maintain stringent stereochemistry, however, non-crystallographic symmetry restraints were ultimately not applied. Strong omit density was apparent at very early stages for G758, LPS (central vestibule), and unassigned detergent-like densities, but these were modeled only at very late stages of refinement. The geometry of the final G758-LPS-MsbA-chim5 model was assessed using MolProbity (Chen et al., 2010). All structural figures were prepared with the PyMol software (The PyMOL Molecular Graphics System v.1.8 (Schrödinger, LLC., 2015)).

c. Cryo-Electron Microscopy (Cryo-EM) Sample Preparation and Data Collection

Purified MsbA was mixed 1:1 w/w with Fab 12G7. The MsbA-12G7 complex was separated from free Fab by size-exclusion chromatography with a running buffer consisting of 20 mM Tris pH 8, 100 mM NaCl and 0.03% αDDM. The MsbA-12G7 complex was incubated with 20 mM MgCl₂, 15 mM ATP and 3.3 mM sodium vanadate for 1 hour on ice. The vanadate trapped sample was further purified by size-exclusion chromatography with a running buffer of 20 mM Tris pH 8, 100mM NaCl, 0.03% aDDM, 20 mM MgCl₂, 15 mM ATP and 1 mM sodium vanadate. Peak fractions were concentrated to 2 mg/mL and incubated with 37 μM G1118 or 67 μM G1365 derivatives (FIG. 14A) and 0.007% Brij-35. After incubating with ligands for 30 minutes on ice, 3.5 μL of each sample was frozen on glow-discharged Ultrafoil 2/2 200 mesh grids using a Vitrobot at 4° C. and 100% humidity. Cryo-EM data was collected on a Titan Krios (ThermoFisher Scientific, Waltham, MA) operated at 300 kV equipped with a BioQuantum energy filter with a 20 eV slit width and a K2 Summit direct electron detector camera (Gatan, Inc, Pleasanton, CA). Images were recorded at a nominal magnification of 165,000× with a physical pixel size of 0.849 Å. Each image stack contains 40 frames with a frame duration of 0.25 s and a total exposure of ˜40 e⁻/Å². Data was collected with a set defocus range of 1.0 to 2.0 μm.

d. Cryo-EM Data Processing for G1118

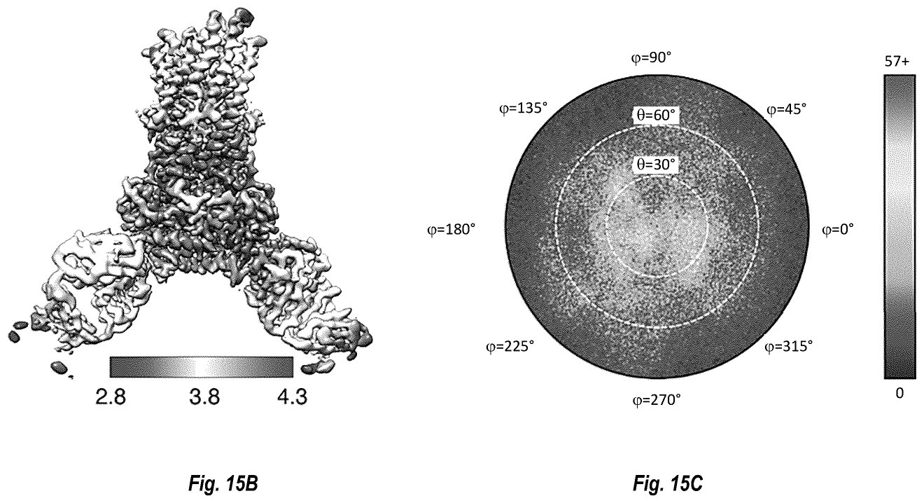

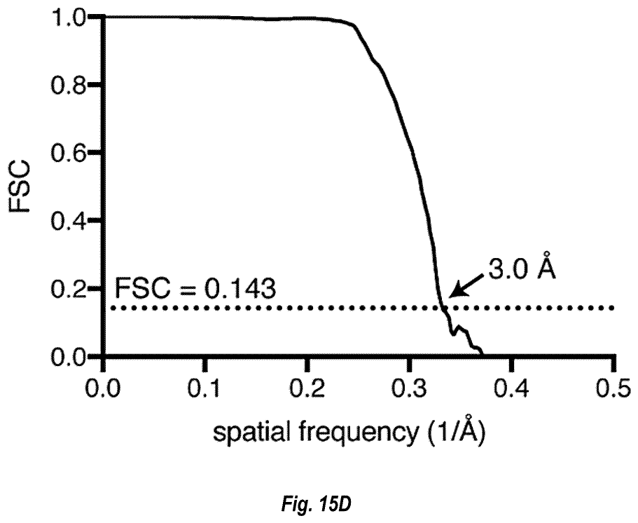

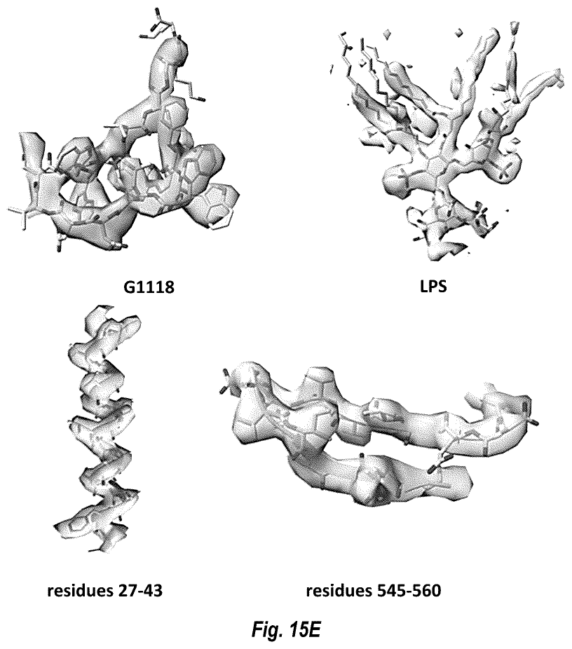

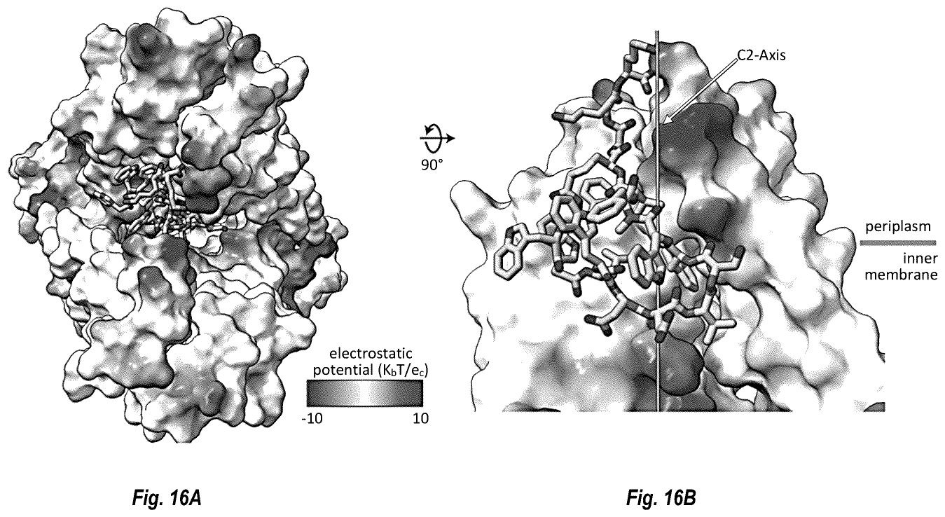

Cryo-EM data for G1118 were processed using cisTEM (Grant et al., 2018) (FIGS. 15A-E). A total of 9,999 movies were corrected for full-frame motion using cisTEM and binned to a pixel size of 1.3 Å. The contrast-transfer function parameters were fit using the 30-4.0 Å band of the spectrum with CTFFIND-4 and 5,984 micrographs with CTF resolutions better than 8 Å were selected for further processing. A total of 1,206,819 particles were picked using cisTEM. The Ab initio model was generated using cisTEM with particles that were well aligned in 2D class averages. The full dataset was refined against the Ab initio model with C1 symmetry. Angular assignments from the consensus refinement were used for 3D classification without alignment. The 410,386 particles from the best class were further manually refined by iteratively increasing the resolution limit used for refinement. Density for the detergent micelle and Fab were filtered to 20 Å through the use of mask around MsbA. Refinement converged to a resolution of 3.0 Å (Fourier shell correlation (FSC)=0.143, determined in cisTEM) with frequencies up to 4 Å used for alignment. For model building and figure preparation, the map was sharpened in cisTEM with the following parameters: flattening from a resolution of 10 Å, applying a pre-cut-off B-factor of −90 Å2 from the origin of reciprocal space and applying a figure-of-merit filter.

e. Cryo-EM Data Processing for G1365

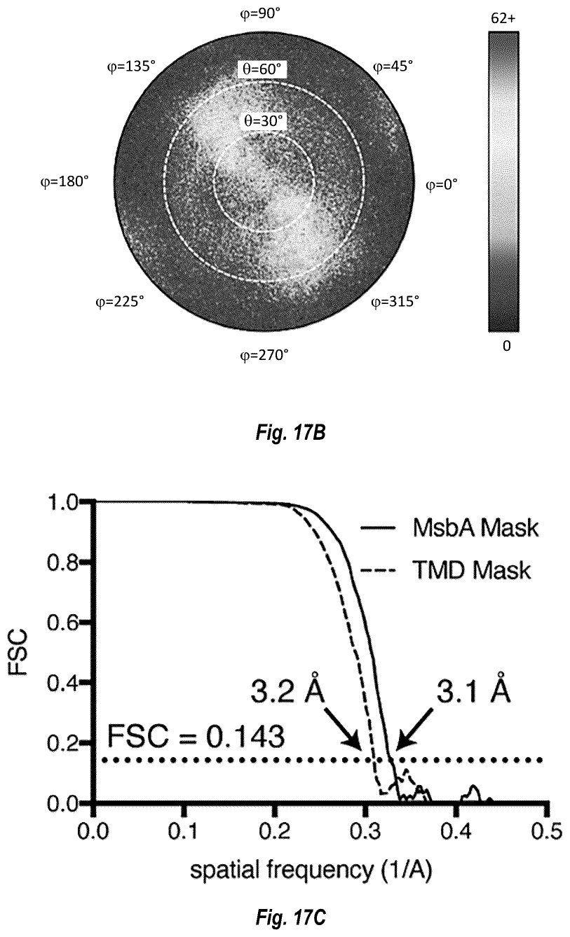

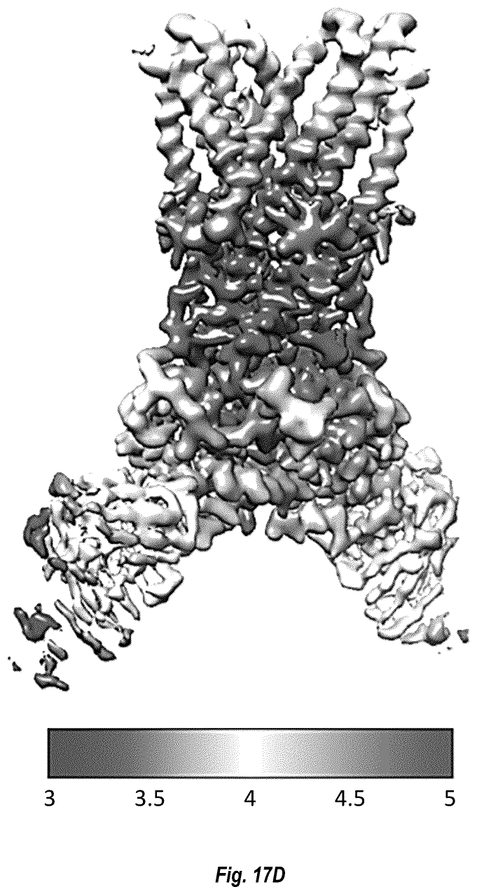

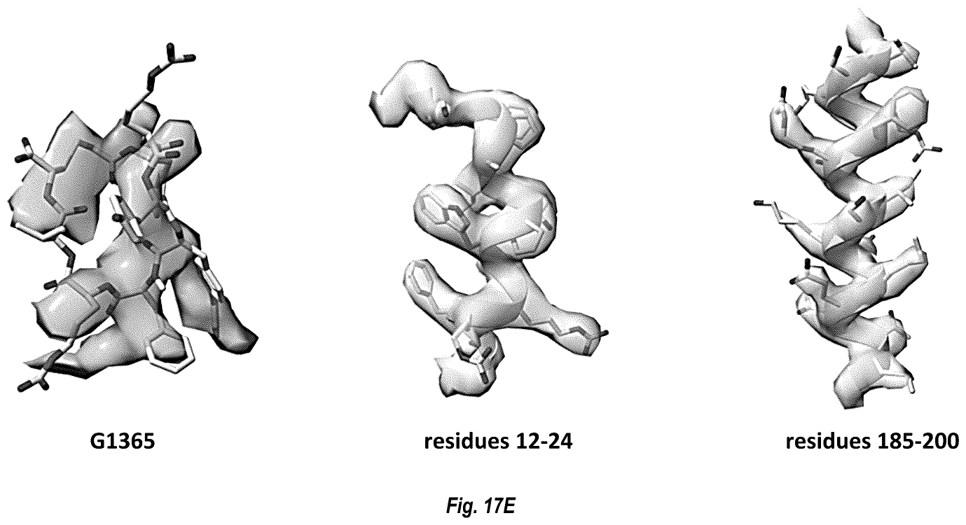

Cryo-EM data were processed using a combination of cisTEM (Grant et al., 2018) and RELION (Scheres, 2012) (FIGS. 17A-E). A total of 10,273 micrographs with G6671 and 17,575 micrographs for G3081 were collected. Each dataset was corrected for full-frame motion using cisTEM and binned to a pixel size of 1.132 Å. The contrast-transfer function parameters were fit using the 30-4.0 Å band of the spectrum with CTFFIND-4. A total of 937,971 particles for G6671 and 993, 151 particles G3081 were picked using cisTEM. Each dataset was individually subjected to a round of 2D and 3D classification in relion. Accurate placement of the macrocycle was not possible due to limited resolution when processing each dataset individually. To increase the averaging power, 218,877 particles from the G3081 dataset and 222,975 particles from G6671 dataset were merged and refinement used in a round of auto-refinement in cisTEM. Density outside of an MsbA mask was filtered to 20 Å. For model building of MsbA an additional round of local refinement was done with a high-res limit of 4 Å yielding a 3.1 Å map (Fourier shell correlation (FSC)=0.143, determined in cisTEM). Additional focused refinement was done with a mask including only the TMD domain of MsbA with a high-res limit of 4.5 Å to aid in macrocycle model building yielding a map 3.2 Å with better density of G1365. For model building and figure preparation, the maps sharpened in cisTEM with the following parameters: flattening from a resolution of 8 Å, applying a pre-cut-off B-factor of either −90 Å2 for the MsbA map or −120 Å2 for the TMD focused refinement and applying a figure-of-merit filter. Electostatic maps were calculated in PyMol with APBS.

f. Model Refinement

A homology model of E. coli MsbA was generated based on the AMPPNP-bound structure of S. typhimurium MsbA (PDB: 3B60) using SWISS-MODEL (Waterhouse et al., 2018). The resulting model was fit as a rigid body into the G1118 cry-o-EM map. The model was refined through manual adjustment in Coot (Emsley & Cowtan, 2004) and ChimeraX (Goddard et al., 2018; Pettersen et al., 2021) with ISOLDE (Croll, 2018) followed by real space refinement in phenix.real_space_refinement (Afonine et al., 2018). The macrocycle was parameterized using Corina (Molecular Networks GmbH) and manually fit into the density. The MsbA coordinates from the G1118 structure were subsequently used as the initial model for G1365 before iterative model building. The model was validated using phenix.validation_cryoEM with built-in MolProbity scoring. Figures were generated using UCSF ChimeraX (Goddard et al., 2018; Pettersen et al., 2021).

### Example 7: Discovery and Characterization of Macrocylic MsbA Inhibitors

a. Comparison of Molecules Selected With and Without Counterselection and Outward-Facing Conformation Trapping (a Preliminary Screen)

To evaluate the ability of different selection schemes to identify 14-mer macrocycle molecules binding selectively to the periplasmic face of MsbA, the results of several selection schemes were compared by ELISA assay (Table 3). Many peptides were used to show specific binding to MsbA. The peptides exhibit both vanadate-dependent binding and no reduction in percent recover in the presence of a chimera.

As shown in Table 3, at top, left, a selection performed (a) in the presence of vanadate to trap EcMsbA in its outward-facing conformation and with a counterscreen using the chimeric MsbA, performed in solution, led to the identification of a group of macrocycles at frequencies of about 26% to 2% (Table 3; % freq column, first 6 rows). A similar set of particular macrocycles was identified when screens were performed (b) with vanadate but with no counterselection, performed in solution, (c) without vanadate but including a counterselection, on beads, and (d) with vanadate, but with no counterselection, on beads. (See far left column and “% freq” column of Table 3.) A subsequent ELISA assay was performed on each of the macrocycles identified to test binding to FcMsbA and to an unrelated membrane protein OprF and to the streptavidin beads (SA) in the presence and absence of vanadate to trap the FcMsbA protein in the outward-facing conformation. The results show that molecules selected in (a) with a counter-screen and with vanadate, in solution, bound well to EcMsbA in the presence of vanadate but poorly without vanadate (“+/−ratio” column, first 6 rows), and did not bind well to either OprF or the streptavidin beads alone under either condition. Most of the molecules in the screens identified in conditions (b), (c), and (d) bound EcMsbA to a similar extent whether it was trapped in the outward-facing conformation with vanadate or not, indicating that both trapping the protein in the outward-facing conformation and using the chimeric protein counterscreen was more efficient in identifying molecules that bind to the periplasmic face of EcMsbA. A competition ELISA experiment was also performed to test binding of the identified molecules to EcMsbA on beads in the presence and absence of the chimeric FcMsbA protein as a binding competitor. A similar degree of binding in the presence and absence of EcMsbA, and thus a ratio greater than or equal to 1.0, indicates that the molecule is selective for EcMsbA and that the chimera does not act as a competitor for binding to FcMsbA. (See the righthand column of Table 3.) Molecules identified under the screening conditions of (a) had a similar degree of binding in the presence and absence of the chimera, as indicated by ratios at or above 1.0 in the assay. This is consistent with binding of those molecules to the periplasmic face of EcMsbA.

b. Site-Directed Discovery of MsbA Inhibitor G1118

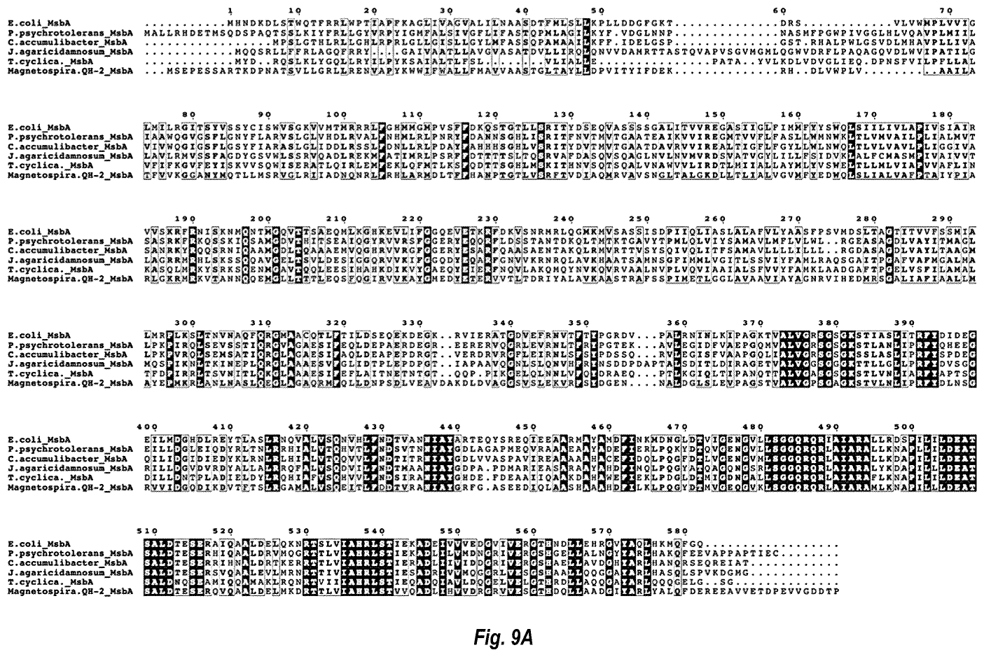

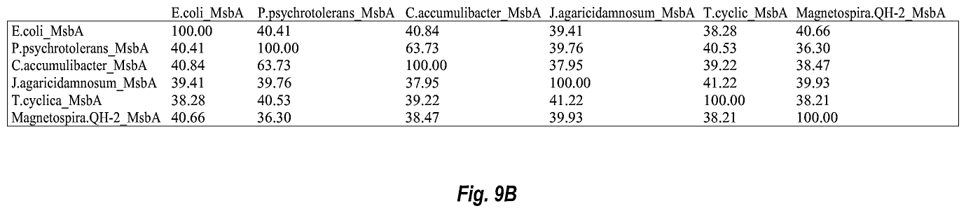

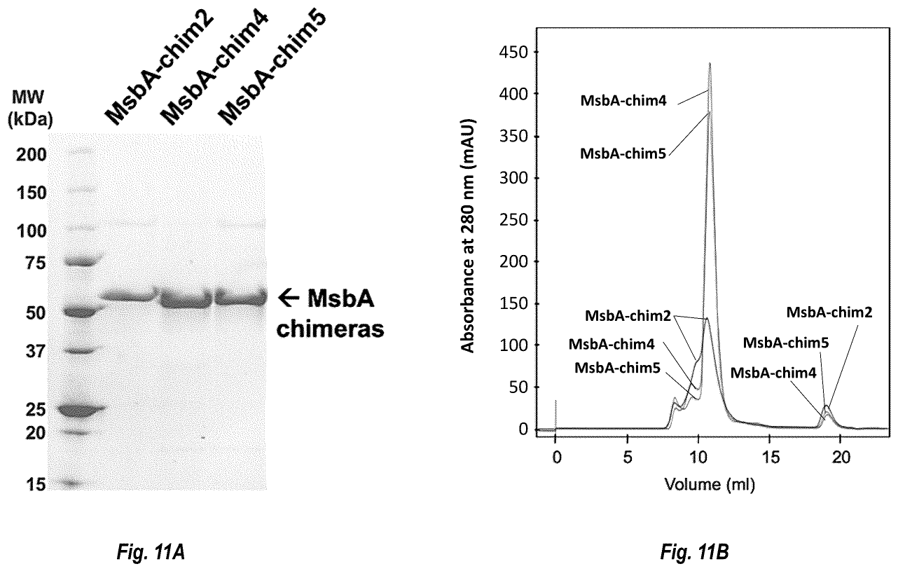

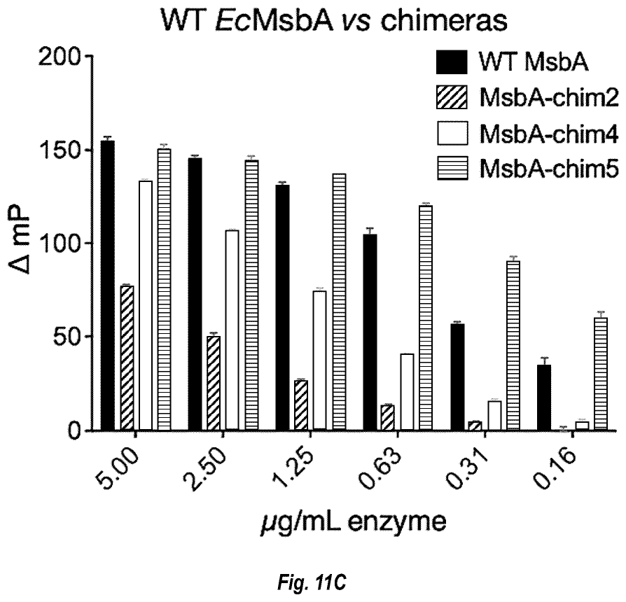

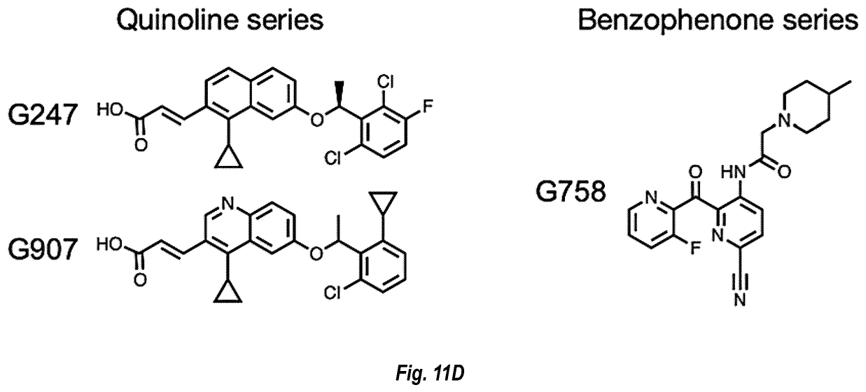

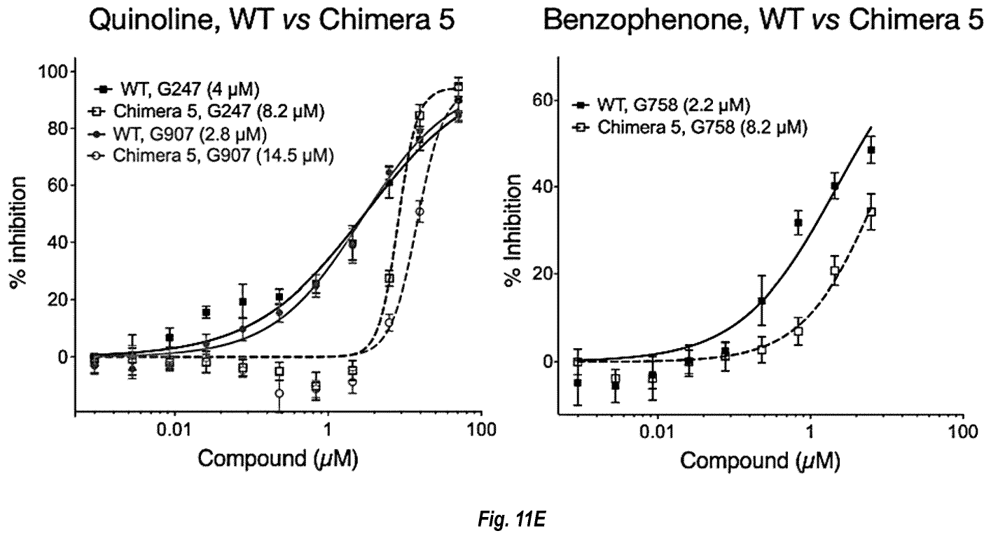

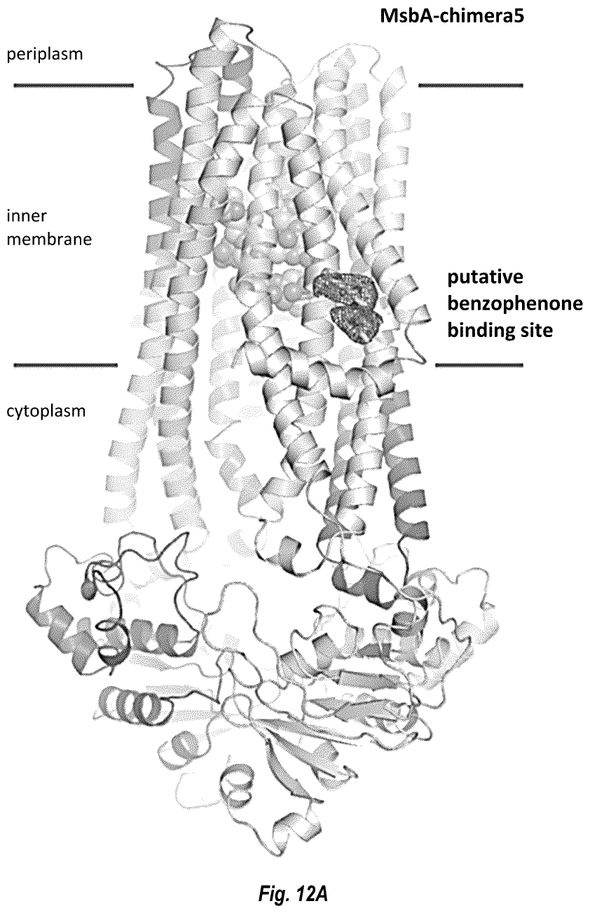

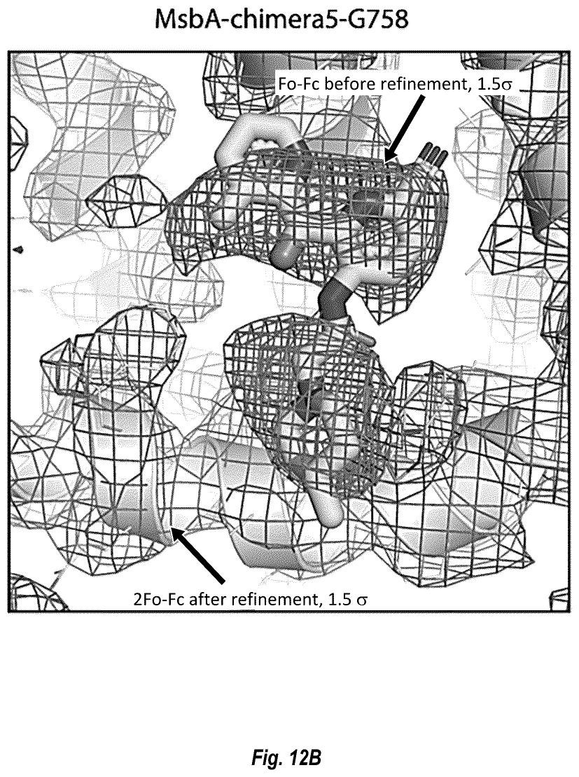

To enrich for molecules that specifically target the aqueous periplasmic cleft of Escherichia coli MsbA (EcMsbA), structure-based homology modeling was first used to guide the engineering of five unique chimeric constructs that each replace the periplasmic face of the transmembrane segments en masse with sequences of putative MsbA transporter proteins from distantly-related Gram-negative bacteria, as assessed using iterative BLAST searches (FIGS. 9A-12B). Of the five chimeras constructed, chimera 2, chimera 4 and chimera 5 (MsbA-chim5) was stably expressed and purified from E. coli (FIGS. 11A-B). Evaluation of the ATPase activity and pharmacology of the chimeras revealed that these large sequence changes could be tolerated while retaining biochemical activity (FIG. 11C). In particular, MsbA-chim5 replaced the entire periplasmic face of EcMsbA with sequences from the MsbA homologue from Magnetospira spirillum, yet retained comparable ATPase activity and displayed sensitivity to both the quinoline (G247 and G907) and benzophenone (G758) classes of previously identified small-molecule inhibitors of wild-type (WT) EcMsbA (FIGS. 11D-E and 12A-B).

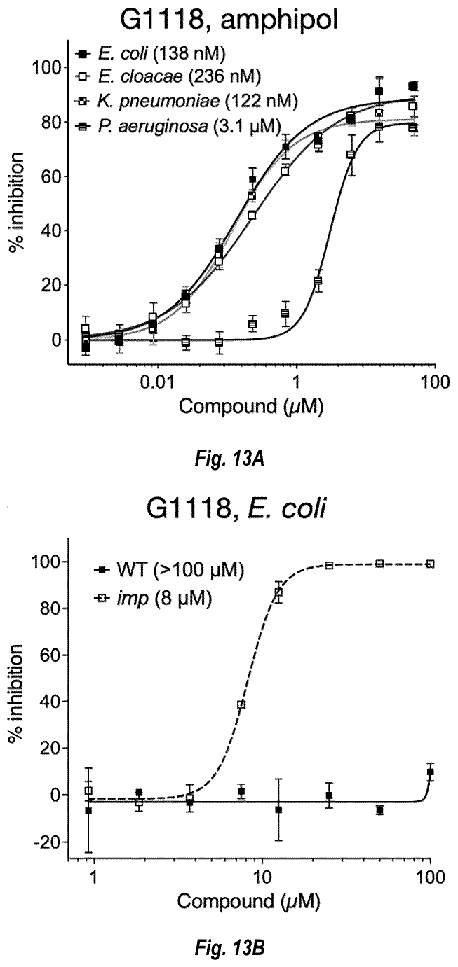

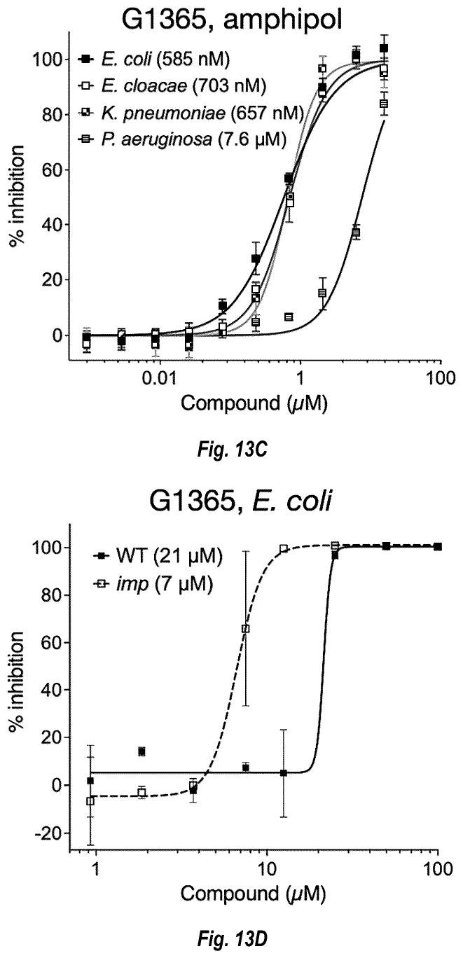

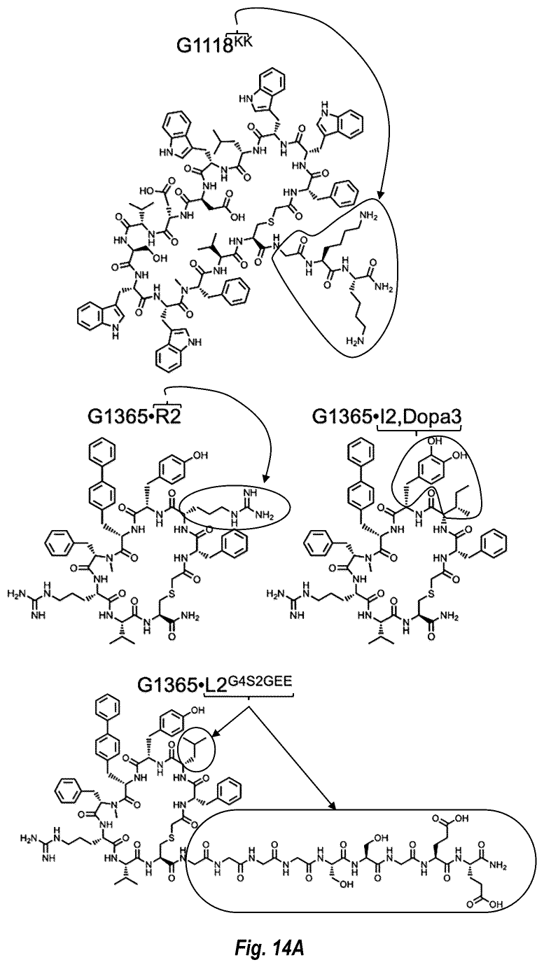

Next, macrocycle selections on purified WT EcMsbA transporter trapped in an outward-facing conformation, followed by a counter-selection using a similarly trapped MsbA chimera in which the periplasmic face of the transmembrane segments was replaced en masse with sequences of putative MsbA transporter proteins from distantly-related Gram-negative bacteria (FIGS. 3A and 9A-12B). Selections employed an in vitro translated peptide macrocycle mRNA display 12-14mer library containing 10 (Karow & Georgopoulos, 1993) molecules that incorporated natural and non-natural amino acids (Example 2). After eight rounds of selection and counter-selection, a 14-mer macrocycle, G1118, showed significant enrichment on WT EcMsbA (FIG. 3B). G1118 was found to impact the ATPase activity of EcMsbA with half-maximum inhibitory concentration (IC₅₀) of ˜110 nM, and displayed >30-fold lower potency on purified MsbA-chim5 (IC₅₀>5 μM), consistent with periplasmic cleft targeting (FIG. 3C). G1118 also inhibited the related Enterobacter cloacae and Klebsiella pneumoniae MsbA transporters with IC₅₀'s of ˜240 and ˜120 nM, respectively (FIGS. 13A-D), but had only weak activity on Pseudomonas aeruginosa MsbA (IC₅₀=3.1 μM), which has lower sequence identity with EcMsbA (˜39%). E. coli cells treated with G1118 gave rise to the distinctive inner membrane elaboration phenotype that mirrors bacteria depleted of MsbA (Doerrler et al., 2001), consistent with pharmacological inhibition of MsbA (FIG. 3E). Due to the relatively large size of G1118 (2,085 Da), cellular activity was limited to cells containing the lptD(imp4213) allele that confers outer membrane (OM) permeability defects (FIG. 13B), with a measured minimum inhibitory concentration (MIC) of ˜210 μg/mL. Further characterization of G1118 is discussed in Example 8 below.

c. Site-Directed Discovery of MsbA Inhibitor G1365

To discover macrocycles with OM permeability and growth inhibition activity on WT E. coli strains, smaller macrocycle scaffolds were identified. These small macrocycle scaffolds also target MsbA at the periplasmic cleft by performing additional selections on EcMsbA and MsbA-chim5 using 8-10 mer macrocycle libraries. Also, a codon table biased for lipophilic and amine-containing side-chains was employed since it is known that small and amphiphilic molecules that contain a primary amine are most likely to accumulate in cells (Richter et al., 2017).

An 8-mer macrocycle, G1365, was discovered. G1365 showed enrichment and ATPase inhibitory activity on WT FcMsbA (IC₅₀=1.3 μM) relative to the MsbA-chim5 transporter (IC₅₀>5 μM) (FIGS. 3B and 3D). In biochemical assays, G1365 inhibited the related Enterobacter cloacae and Klebsiella pneumoniae MsbA transporters with IC₅₀'s of ˜700 and ˜660 nM, respectively (FIG. 13C), but had more modest inhibitory activity on the distantly related Pseudomonas aeruginosa MsbA (IC₅₀=7.6 μM). G1365 treatment of E. coli imp4213 mirrored the inner membrane elaboration phenotype of bacteria depleted of MsbA, consistent with on-target MsbA activity (FIG. 3E). In contrast to the larger 14-mer macrocycle G1118, G1365 was found to inhibit the growth of WT E. coli cells with an intact outer membrane over the course of 6 hours with an IC₅₀ of ˜20 μM (FIGS. 13B and 13D), although an overnight MIC could not be determined. Whole-genome sequencing was subsequently performed on an isolated E. coli imp4213 mutant resistant to a derivative of G1365 (see Methods) that revealed a single nucleotide mutation in msbA resulting in an amino acid change within the periplasmic cleft of MsbA, Asp252Asn. Further characterization of G1365 is discussed in Example 9 below.

The data collectively identify MsbA as the cellular target of G1365 and highlight the potential to discover synthetic macrocycles that can cross an intact bacterial outer membrane.

d. Additional 14-mer Macrocyclic Peptide Activators

Additional macrocyclic peptide activators tested were G1119 (ClAc-FWWLWSDMeGDWWMeFVC-NH2; SEQ ID NO: 10) and G1122 (ClAc-FRYLWMeAWGLVWDNC-NH2; SEQ ID NO: 11).

Cryo-EM analysis of G1119 and G1122 showed that they cause stacking of extracellular membranes to an even greater extent than G1118 (data for G1119 and G1122 not shown; see FIG. 3F for G1118 data). An IC₅₀ for inhibition of ATPase activity of MsbA by G1118, G1119 and G1122 was performed (see Example 8 for method) and IC₅₀ values of 5 nM for G1118 and G1119 and of 2 nM for G1122 were determined.

Eleven different molecules designed according to the formula ClacF-X1-X2-L-X3-X4-D-X5-X6-X7-X8-MeF-V-C (SEQ ID NO: 17; see Example 5 above) were also tested for inhibitory effects on MsbA. ATPase activity, in an assay as described in Example 3 above, and found to have IC₅₀ values of 0.2 to 0.8 μM. The minimum inhibitory concentration (MIC) for inhibition of E. coli MsbA (EcMsbA) (see Example 3 for methods) was more than 100 μM, and the EC₅₀ for inhibition of growth of cells of a UPEC imp strain was from about 2 to about 8 μM for all but one of the tested molecules (and over 100 μM for the final tested molecule). Collectively, these data indicate that macrocyclic peptides G1118 and related peptides according to the formula above are generally able to inhibit MsbA and cellular activity related to MsbA.

### Example 8: G1118 Binds to MsbA

a. Biochemical Characterization of G1118

Using materials and methods described in Examples 1-5 above, G1118 was identified as macrocycle molecule that binds MsbA. A 14-residue peptide macrocycle, G1118 was identified after eight rounds of enrichment and screening. G1118 displayed potent inhibitory activity on EcMsbA (half-maximum inhibitory concentration (IC₅₀) of ˜110 nM), yet weak activity on the counter-selection chimera (IC₅₀>5 μM; FIGS. 3B-C and 13A). G1118 treatment of outer membrane permeable E. coli cells bearing the IptD(imp4213) allele (Sampson et al., 1989) mirrored the distinctive inner membrane elaboration phenotype that is characteristic of MsbA depletion² (FIG. 3E), however no activity was observed on WT E. coli with an intact outer membrane. G1118 is a 14-mer macrocycle compound that was identified from the macrocycle selections on purified WT transporter trapped in an outward-facing conformation by treatment with Mg²⁺, ATP and vanadate, followed by a counterselection using similarly trapped periplasmic chimera to remove macrocycles binding to regions other than the periplasmic cleft. G1118 has potent inhibitory activity on EcMsbA. After the counter-selection with the chimeric MsbA, G1118 continued to show significant enrichment on WT EcMsbA, and its inhibitory activity was consistent with targeting the periplasmic cleft, since G1118 displayed >30-fold lower potency on the purified chimera. E. coli treated with G1118 also mirrored the distinctive membrane elaboration phenotype of bacteria depleted of MsbA, consistent with on-target activity. However, G1118 cellular activity was limited to cells also containing the imp4213 allele that confers defects in outer membrane (OM) permeability, suggesting that G1118 has difficulty in crossing the OM to reach the MsbA protein. As G1118 represents the first potent and selective inhibitor of MsbA targeting the periplasmic cleft, the molecular basis for its antagonism was studied using structural biology methods as described in Example 6 above.

b. G118 Binds to MsbA at a Periplasmic Binding Site and Traps MsbA in an Outward-Facing State

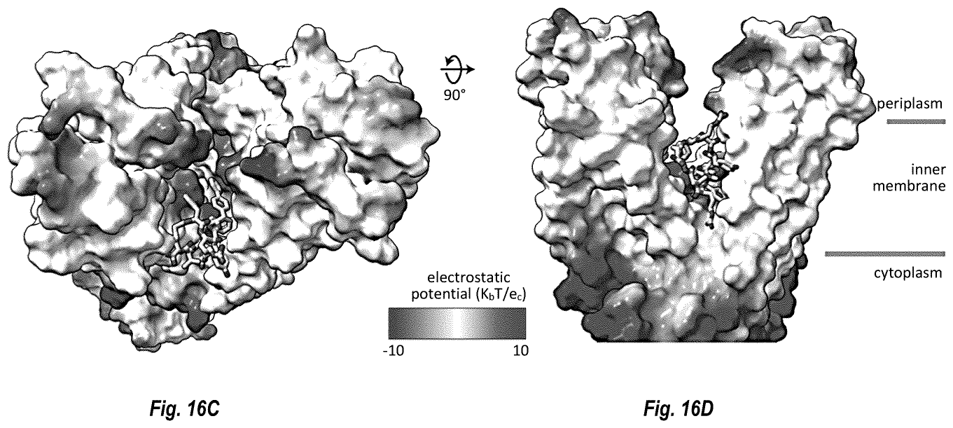

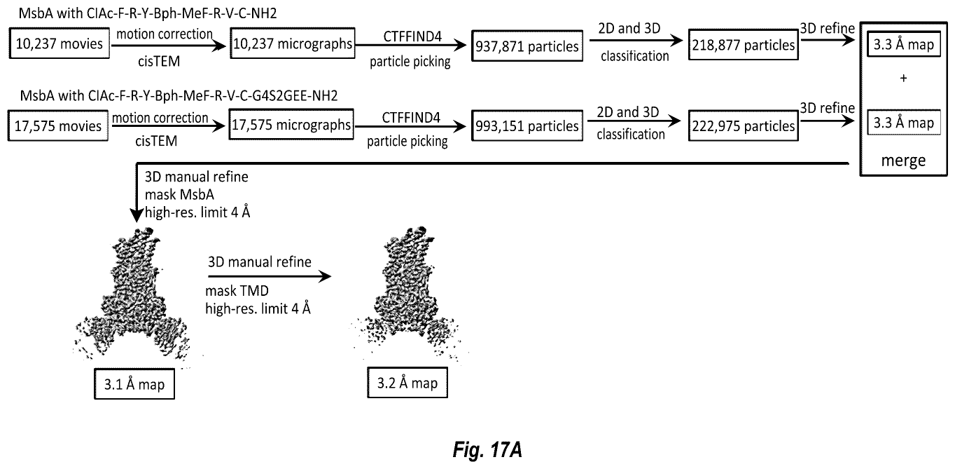

To define the molecular mechanism by which G1118 antagonizes MsbA a 3.0Å cryo-EM structure of the MsbA-G1118 complex in the presence of ADP-vanadate was determined using an antigen-binding fragment of an antibody (Fab) to aid in particle alignment (FIGS. 4A-D, 14A-C, 15A-E, and 8). Materials and methods were as described in Example 6 above. In complex with G1118, the MsbA homodimer adopts an outward-open conformation with a solvent-exposed periplasmic pocket formed between transmembrane helices TM1, TM3 and TM6 of each MsbA subunit (FIGS. 4A-D). Strong features in the cryo-EM map informed of the presence of a single G1118 macrocycle in the periplasmic cleft, validating the design of the chimeric selection strategy. G1118 is an amphipathic peptide that complements the complex physicochemical properties of the MsbA periplasmic cleft, making complementary polar, van der Waals and hydrophobic contacts and burying ˜880 A² of solvent accessible surface area (FIGS. 16A-B). G1118 binding is incompatible with the rearrangements necessary for MsbA to return to the inward-facing conformation (Ho et al., 2018; Mi et al., 2017; Ward et al., 2007), defining this macrocycle as a state-dependent inhibitor that locks the transporter in the outward-facing conformation to prevent LPS transport. As G1118 was observed to cause cell killing by inhibiting MsbA (FIG. 3E), the co-complex structure likely represents the physiologically relevant inhibitor complex.

The periplasmic receptor site for G1118 on MsbA is found near the central axis of the transporter, extending above and below the membrane-aqueous interface (FIGS. 4B, and 16A-B). The MsbA dimer is highly symmetric (˜0.4 Å Cα RMSD), and G1118 exploits this intrinsic symmetry by making pseudosymmetric interactions using the indole rings of Trp10^{G1118} and Trp11^{G1118} to pack between Leu45, Leu52, and Ile292′ on either side of the transporter between TM1 and TM6′ (FIG. 4C). Ser9^{G1118} and Trp3^{G1118} form a pair of hydrogen bonds to MsbA Lys49 and the backbone carbonyl of Ser289′, respectively (FIG. 4D). On the polar face of G1118, a salt bridge is observed directly between Asp7^{G1118} and Arg296, which is a highly conserved basic residue deep within the central cavity of MsbA previously seen to coordinate LPS in the inward-facing conformation (FIG. 4C) (Ho et al., 2018; Mi et al., 2017). Analysis of enriched related sequences and a mutational scan of G1118 confirms the key interactions and amphipathic nature of the macrocycle required to interact with MsbA (FIG. 4E and Table 4).

Table 4 above shows a mutational scan of macrocycle G1118. The enrichment factor is shown for each macrocycle containing the indicated substitutions at positions 2-13 of the macrocycle core (where enrichment factor=(% Frequency[target]/% Frequency[input]), normalized by the parent value). Amino acids identical to the parent are indicated with a black outline. Natural amino acids are indicated by standard single letters. Non-natural amino acids are as follows: (1) MeF=F*, N-Methyl-L-phenylalanine; (2) MeG=G*, N-Methyl-L-glycine; (3) Dopa, L-3,4-dihydroxyphenylalanine; and (4) Bph=B*, (S)-3-([1,1′-biphenyl]-4-yl)-2-aminopropanoic acid. Overall, our biochemical and structural studies identify a novel pharmacology and receptor site within the ABC transporter family.

In summary, the structure of the complex guided choice residues for mutational analysis. IC₅₀ in vitro curves showed that these residues impaired macrocycle binding. Reciprocally, analysis of the enriched sequences from the RNA-display selection pointed to the role of residues on the macrocycle for affinity. Overall, the structure and cellular activity validated the macrocycle selection strategy and the periplasmic cleft as a druggable location critically validating our protein engineering and selection strategy. The large size of G1118 hampered optimization of derivatives with wild-type activity likely due to challenges penetrating the LPS rich outer membrane.

### Example 9: Identification of G1365

a. Biochemical Characterization of G1365

In an attempt to select for smaller macrocycles that could overcome the OM permeability barrier, another series of selection and chimera counter-selection was performed using 8-10 mer macrocycles. Materials and methods were as described above in Examples 1-5. A codon table biased for lipophilic and positively charged side-chains was also used.

With these efforts, G1365 was identified (Example 7 and FIG. 3B). G1365 displayed biochemical activity on MsbA (FIG. 3D; IC₅₀=1.3 μM), and E. coli lptD(imp4213) cells treated with G1365 also revealed the MsbA depletion membrane phenotype (FIG. 3E). Notably, G1365 also inhibited the growth of WT E. coli cells with an intact outer membrane over the course of 6 hours (IC₅₀ of ˜20 μM; FIGS. 13B and 13D), highlighting the potential to discover synthetic macrocycles that can cross an intact bacterial outer membrane. Specifically, the effect of G1365 and other macrocycles on the viability of imp E. coli cells expressing normal and high levels of MsbA (MsbA WT and MsbA High and on wild-type E. coli cells (UPEC WT; i.e., with a normal OM) was determined at different concentrations of the macrocycles (FIGS. 3E and 3G-L, and Table 5).

TABLE 5
imp Strain MICs.

| Compound | imp, E.coli MsbA | imp A. bau. MsbA |
|---|---|---|
| G1320 | >100 | >100 |
| G1323 | >100 | >100 |
| G1330 | 3.13 | 3.13 |
| G1325 | 12.50 | 12.50 |
| G1349 | 6.25 | 25.00 |
| G1365 | 12.5 | >100 |

Bold indicates compound and E. coli strain with phenotype shown at FIG. 3E, right-most panel.

The biochemical IC₅₀ for G1365 for this experiment was 300 nM and the EC₅₀ for wild-type E. coli cells was 20 μM. See FIGS. 3G-L and Table 5 below. To further verify that G1365 is selective for the MsbA from E. coli, the minimum inhibitory concentration (MIC) of G1365 and other macrocycles for growth inhibition of imp E. coli and of a different bacterial strain, Acinetobacter baumanni (A. bau.) was determined. The MIC of G1365 for imp E. coli was 12.5 μM, while that for A. bau. was over 100 μM, about a 10-fold difference in concentration, indicating that G1365 interacts preferentially with the MsbA of E. coli compared to the MsbA of A. bau. The MIC for the other tested macrocycles did not differ between the two bacterial strains, indicating that they are not selective between the two MsbA proteins. The molecular basis for its antagonism was studied using structural biology methods as described in Example 6 above.

b. G1365 Binds Deep Within the Periplasmic Cleft of MsbA

To understand the molecular basis of inhibition, a cryo-EM structure of the MsbA-12G7-G1365 complex was determined. Materials and methods were as described in Example 6 above. A 3.1 Å cryo-EM structure of MsbA in complex with potent derivatives of G1365 (FIGS. 14A-B, 17A-E, and 8). Notably, G1365 is an amphipathic molecule and the smaller size of G1365 allows it to bind almost 10 Å deeper in the periplasmic cleft of MsbA compared to G1118 (FIGS. 4A, 4B, 4F, and 16A-D). Unlike G1118, which binds along the central axis of MsbA, the G1365 receptor site is found on the edge of the periplasmic cleft (FIGS. 4A, 4B, 4F-K, and 16A-D), suggesting that this amphipathic macrocycle might directly access the receptor site from the lipid bilayer.

G1365 traps MsbA in an outward-facing conformation that is incompatible with transition to an inward-facing state, identifying G1365 as a state-dependent inhibitor. The binding site for G1365 is adjacent to the membrane-exposed region of the transporter with the binding site formed by TM1, TM3, and TM6, and the guanidinium side chain of Arg6^{G1365} points towards the lipid bilayer (FIGS. 4F-H). Cation-π interactions between MeF5^{G1365} and Arg148, and Bhp4^{G1365} with Arg296, respectively, stabilizing binding of G1365 within the receptor site. The extended biphenyl residue Bhp4^{G1365} reaches across the symmetry axis of MsbA, likely precluding binding of a second macrocycle. Enriched related sequences and a mutational scan of G1365 confirm the key interactions of the macrocycle required to interact with MsbA (FIG. 4I and Table 5).

Table 6 above shows a mutational scan of macrocycle G1365. Just as in Table 4 above, the enrichment factor for macrocycles containing the indicated substitutions at positions 2-7 of the macrocycle core (where enrichment factor=(% Frequency[target]/% Frequency[input]), normalized by the parent value). Amino acids identical to the parent are indicated with a black outline. Natural amino acids are indicated by standard single letters. Non-natural amino acids are as follows: (1) MeF=F*, N-Methyl-L-phenylalanine; (2) MeG=G*, N-Methyl-L-glycine; (3) Dopa, L-3,4-dihydroxyphenylalanine; and/or (4) Bph=B*, (S)-3-([1,1′-biphenyl]-4-yl)-2-aminopropanoic acid. The binding pose of G1365 can also rationalize the Asp252Asn resistance mutation (Example 7 above), since this should disrupt the interaction between Arg296 and G1365 (FIG. 4H).

Unexpectedly, the guanidinium side chains of G1365^{Arg6} points towards the lipid bilayer. The bulky biphenyl residue reaches across the pseudosymmetry axis towards Arg296 from the other protomer. The structure also rationalizes the mechanism for the D252N resistance mutation by breaking a salt bridge between Lys299 on TM6 likely disrupting the interaction between Arg296 and G1365. The structure of G1365 identifies a distinct receptor site compared to G1118, even though the outward-open conformation of MsbA that is trapped by the two molecules is highly similar. Thus, like G1118, G1365 is a state-dependent inhibitor of MsbA, despite its smaller binding footprint.

### Example 10: Identification of an Unexpected LPS Binding Site on MsbA

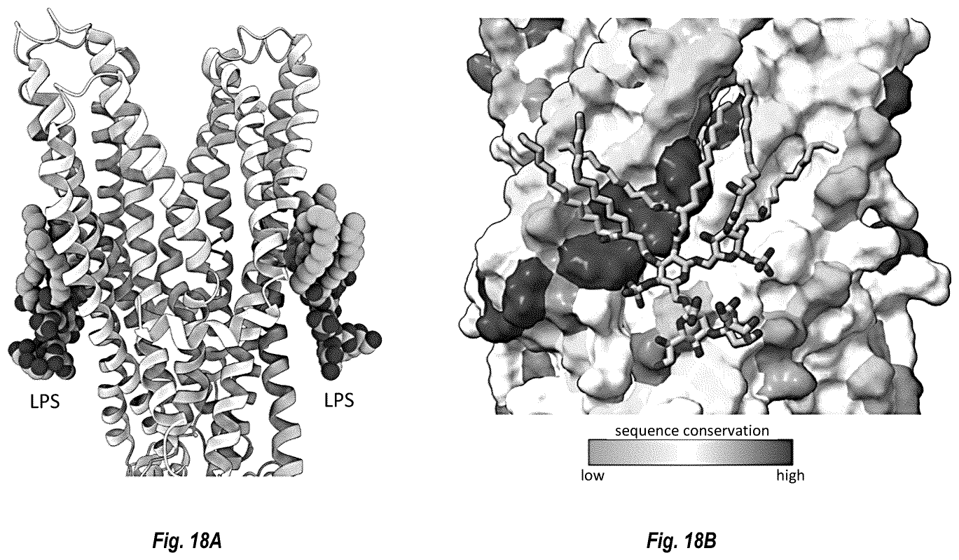

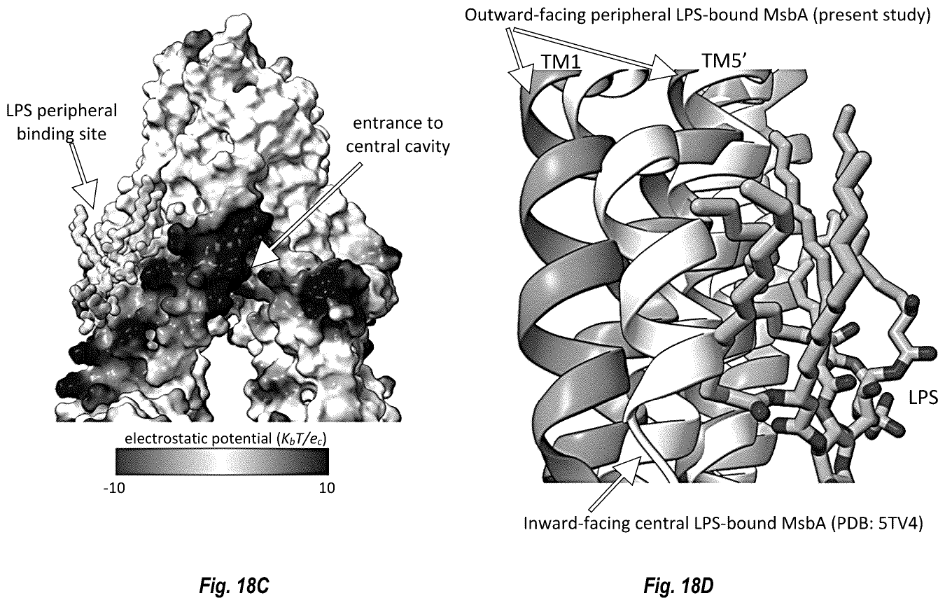

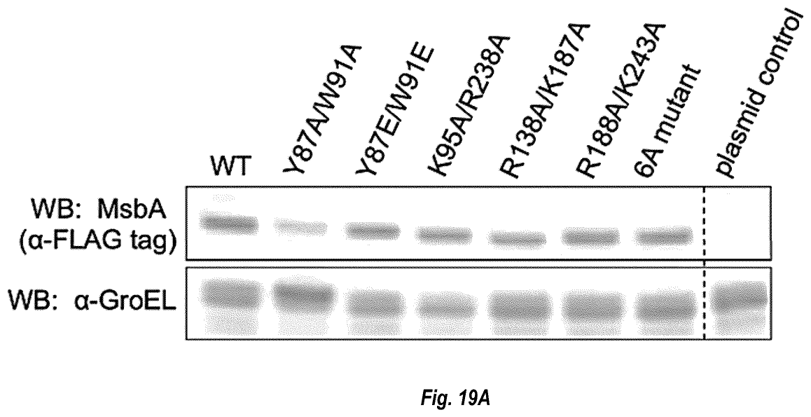

It is now known how MsbA first engages its scarce substrate LPS from the abundant phospholipids within the membrane bilayer to achieve selective transport. In the cryo-EM maps, two well-resolved LPS molecules are bound to the periphery of MsbA (FIGS. 4A and 18A-D). These striking map features unambiguously place the characteristic bi-phosphoglucosamine headgroup of lipid A at previously unrecognized, symmetry-related coordination sites at the level of the inner membrane leaflet on the MsbA dimer (FIG. 18A). Importantly, the visualized LPS molecules are far removed from the single binding site observed previously within the central vestibule of the inward-facing state of MsbA (Ho et al., 2018; Mi et al., 2017). As LPS was not added to the samples used for cryo-EM, the identification of these unique peripheral LPS-binding sites on the outward-facing state of MsbA suggest that they are physiologically relevant

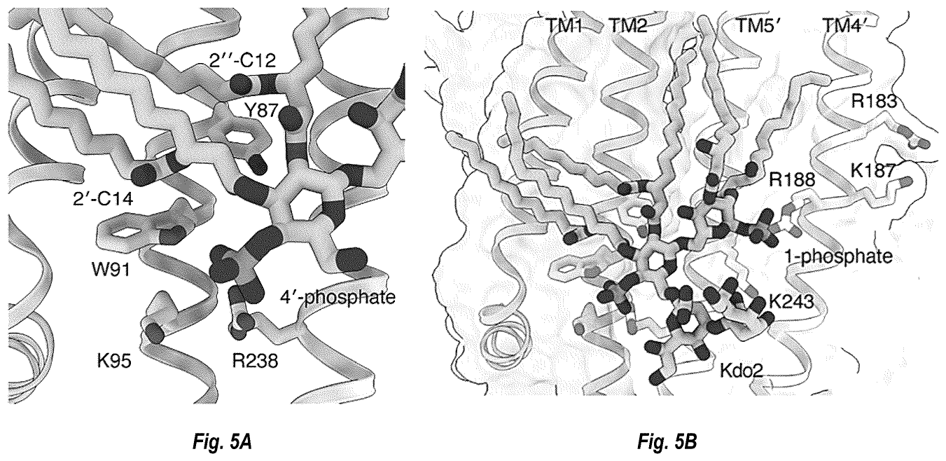

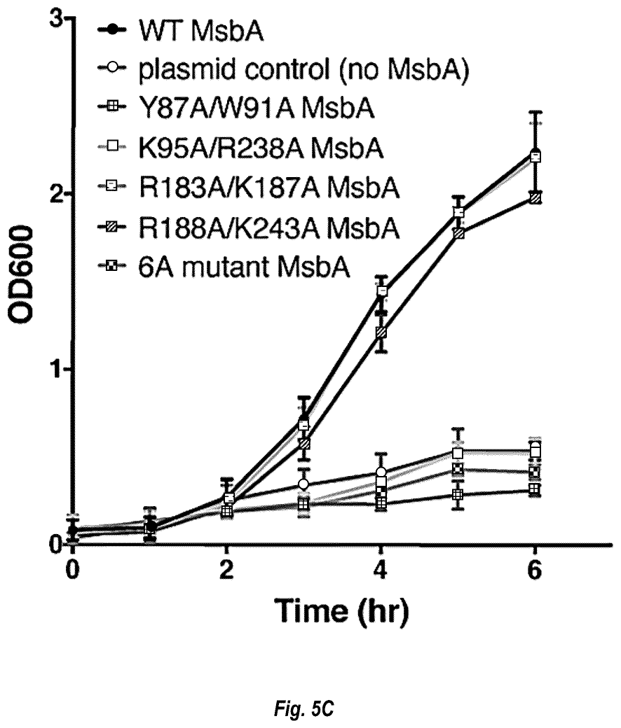

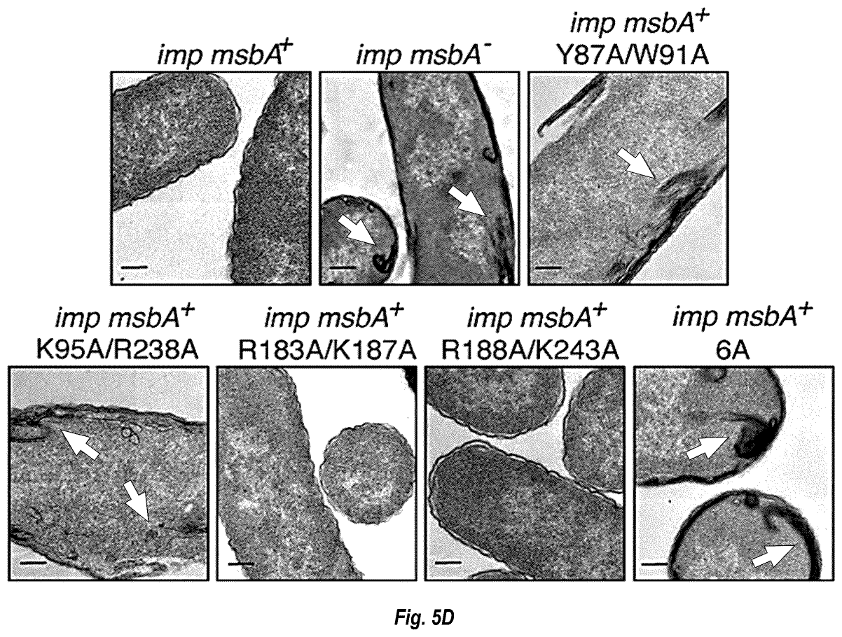

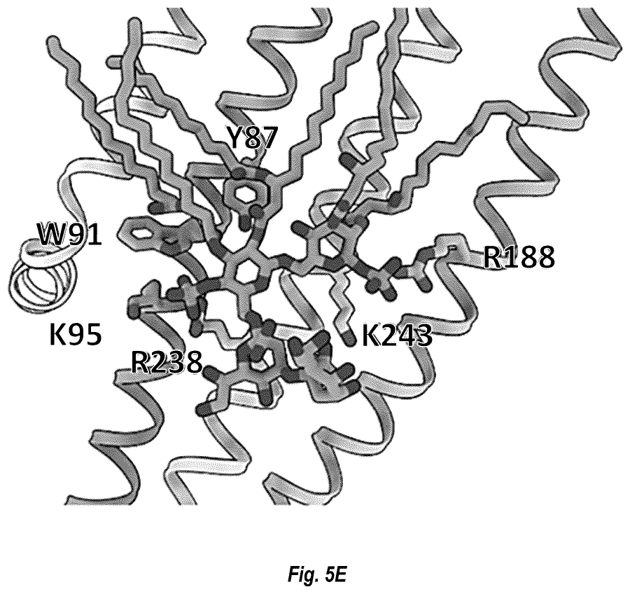

The peripheral inner membrane leaflet LPS-binding site on MsbA is formed by TM2, TM4′ and TM5′ with 865 Å² of transporter surface area is buried upon LPS binding. Three distinguishing features of LPS engaged by MsbA are spread along the lipid A core of the molecule (FIG. 5A). First, highly conserved basic side-chains from MsbA coordinate the characteristic phosphates on the glucosamine headgroups of lipid A (FIG. 18B). Arg188 and Lys243 salt-bridge to the 1-phosphate (FIG. 5A), while the Lys95 and Arg238 form salt-bridges to the 4′-phosphate (FIG. 5B). Second, the hexa-acylated tails of lipid A interact with an extended hydrophobic surface on the transporter, where the 2′-hydroxymyristate and 2″-laurate ester linkages pack against the conserved aromatic side chains of Tyr87 and Trp91, respectively. Third, the elaborated 3-deoxy-D-manno-oct-2-ulosonic acid (Kdo) core-sugars from lipid A are coordinated by an extended polar intracellular stalk region on the transporter (FIG. 18C). These strategic multi-point contacts made to lipid A at the peripheral LPS binding site define a structural basis for MsbA to selectively engage LPS over the bulk membrane phospholipids.

Since the outward-facing conformation of MsbA has historically been associated with substrate release into the periplasmic membrane leaflet (Ho et al., 2018; Mi et al., 2017; Ward et al., 2007), it was initially surprising to find LPS bound to MsbA along the inner leaflet in this state. Targeted double-mutations of residues that interact with LPS were generated. The double-mutants were assessed for the ability to rescue the growth of E. coli cells lacking WT MsbA (FIGS. 19A-J). The basic residues coordinating the 4′-phosphate of lipid A (Lys95 and Arg238) and the aromatic side-chains supporting the 2′-hydroxymyristate and 2″-laurate chain esters of lipid A (Tyr87 and Trp91) were found to be essential. This is because their mutation to alanine arrested cell growth and produced the distinctive inner membrane elaboration phenotype that mirrors bacteria depleted of WT MsbA (FIGS. 5C-E) (Doerrler et al., 2001). By contrast, side-chains coordinating the 1-phosphate of lipid A (Arg188 and Lys243) or proximal non-LPS-interacting residues were dispensable. Unexpectedly, (1) an essential peripheral LPS binding site was discovered on the inner leaflet of MsbA in the outward-facing conformation, and (2) previously unknown substrate selectivity determinants that have important physiological implications were identified.

### Example 11: Discussion

The present disclosure discusses a site-directed discovery of potent and state-dependent inhibitors of an ABC transporter. The present disclosure discusses tools and new findings related to MsbA's mechanism of action. With these tools, many challenges related to the unbiased discovery of ligands that bind to integral membrane receptor sites were overcome.

Using engineered chimeric proteins, a site-directed ligand discovery strategy was devised to selectively inhibit an ABC transporter by targeting the solvent-exposed cleft of the outward-facing state. G1118 and G1365 are two macrocycle compounds that target unprecedented receptor sites within the outward facing cleft, defining a new pharmacology within the ABC transporter superfamily, where these macrocycles may represent early leads for further antibacterial discovery. Analogous site-directed ligand discovery efforts using engineered chimeras could be employed to identify new receptor sites in other protein classes and may be particularly useful to discover novel pharmacologies.

Although prior studies have shed light on the alternating access mechanism of transport employed by MsbA (Ho et al., 2018; Mi et al., 2017; Ward et al., 2007), how MsbA achieves selectivity for LPS over abundant membrane phospholipids remained unknown. Inward-facing structures of MsbA revealed LPS bound in the central cavity, but these structures capture an intermediate state in the transport cycle and fail to provide insight into how LPS is selectively loaded from the bulk phospholipid bilayer into the central cavity. The present disclosure discusses the discovery of MsbA in an outward-facing state bound to its substrate, unexpectedly revealing that MsbA first recognizes LPS through a conserved and essential side-chain network that is positioned along the inner membrane leaflet of the transporter. On the basis of these observations, several principles that may underlie the mechanism of LPS selectivity by MsbA are discussed below.

Until now, the outward-facing conformation in ABC exporters has been strictly associated with substrate release and the end of the catalytic cycle (Hollenstein et al., 2007). As discussed in the Examples above, the data invoke a more holistic model whereby the LPS binding site presented by the outward-facing state would serve to substantially increase the local concentration of LPS within the inner leaflet around the transporter (FIG. 6). In the outward-facing conformation, MsbA strategically targets the distinct chemistry of core-lipid A at the peripheral site through multi-point interactions with the characteristic bi-phosphoglucosamine headgroup, the hexa-acylated chains, and the Kdo-core sugars to achieve selective binding for LPS over phospholipids (FIGS. 5A-B). Targeted mutagenesis and physiological studies established the essential nature of the conserved residues at this previously unrecognized LPS binding site. Peripheral binding site disruption upon transition to the inward-facing conformation is expected to release LPS and facilitate selective loading into the central cavity, presumably along a set of basic residues that stretch from the peripheral binding site to the vestibule (FIGS. 18C-D). In summary, MsbA couples LPS release into the outer leaflet with substrate binding at the inner leaflet, revealing that the outward-facing state previously regarded as the last step in the ABC exporter cycle may be intrinsically and directly linked to the first.

Visualization of the peripheral LPS-recognition complex provides the first insights into how MsbA may discriminate between mature and immature LPS species. Lys95 and Arg238 side-chains are essential and form direct salt-bridges to the 4′-phosphate of lipid A (FIG. 5B, offering a structural rationale for why the precursor 1-mono-phosphorylated lipid A species is not transported23. The essential nature of the Lys95 and Arg238-mediated interactions also explains why suppressor mutations of the 4′-kinase LpxK have not been identified, and rationalizes why MsbA and LpxK are chromosomally linked24 or exist as a single concatenated protein in some bacteria25. Furthermore, the Tyr87 and Trp91 side-chains are essential and directly underlie the ester linkages of the 2′-hydroxymyristate and 2″-laurate acyl chains (FIG. 5B), which are added by late-acting enzymes in the lipid A biogenesis pathway, LpxL and LpxM26,27. This observation provides a molecular rationale for why hexa-acylated LPS is the preferred substrate for EcMsbA28,29. The picture that emerges from the peripheral LPS-recognition site is that, in addition to selectively concentrating LPS over phospholipids, MsbA employs intrinsic quality controls to ensure mature LPS species are selected for transport across the inner membrane into the periplasm. This level of molecular sophistication will help to ensure that the integrity of the outer membrane barrier is not compromised by the passage of immature LPS species. Overall, our findings shed light on the selective recognition and transport of lipids, and introduce the paradigm that ABC transporters can utilize an outward-facing conformation to selectively enrich and interrogate potential substrates.

### REFERENCES

Adams, P. D., Afonine, P. V., Bunkóczi, G., Chen, V. B., Davis, I. W., Echols, N., Headd, J. J., Hung, L.-W., Kapral, G. J., Grosse-Kunstleve, R. W., McCoy, A. J., Moriarty, N. W., Oeffner, R., Read, R. J., Richardson, D. C., Richardson, J. S., Terwilliger, T. C., & Zwart, P. H. (2010). PHENIX: A comprehensive Python-based system for macromolecular structure solution. Acta Crystallographica. Section D, Biological Crystallography, 66(Pt 2), 213-221. https://doi.org/10.1107/S0907444909052925
Afonine, P. V., Poon, B. K., Read, R. J., Sobolev, O. V., Terwilliger, T. C., Urzhumtsev, A., & Adams, P. D. (2018). Real-space refinement in PHENIX for cryo-EM and crystallography. Acta Crystallographica. Section D, Structural Biology, 74(Pt 6), 531-544. https://doi.org/10.1107/S2059798318006551
Alexander, M. K., Miu, A., Oh, A., Reichelt, M., Ho, H., Chalouni, C., Labadie, S., Wang, L., Liang, J., Nickerson, N. N., Hu, H., Yu, L., Du, M., Yan, D., Park, S., Kim, J., Xu, M., Sellers, B. D., Purkey, H. E., . . . Nishiyama, M. (2018). Disrupting Gram-Negative Bacterial Outer Membrane Biosynthesis through Inhibition of the Lipopolysaccharide Transporter MsbA. Antimicrobial Agents and Chemotherapy, 62(11), e01142-18. https://doi.org/10.1128/AAC.01142-18
Altschul, S. F., Gish, W., Miller, W., Myers, E. W., & Lipman, D. J. (1990). Basic local alignment search tool. Journal of Molecular Biology, 215(3), 403-410. https://doi.org/10.1016/S0022-2836(05)80360-2
Brown, E. D., & Wright, G. D. (2016). Antibacterial drug discovery in the resistance era. Nature, 529(7586), 336-343. https://doi.org/10.1038/nature17042
Chen, V. B., Arendall, W. B., Headd, J. J., Keedy, D. A., Immormino, R. M., Kapral, G. J., Murray, L. W., Richardson, J. S., & Richardson, D. C. (2010). MolProbity: All-atom structure validation for macromolecular crystallography. Acta Crystallographica. Section D, Biological Crystallography, 66(Pt 1), 12-21. https://doi.org/10.1107/S0907444909042073
Clementz, T., Bednarski, J. J., & Raetz, C. R. (1996). Function of the htrB high temperature requirement gene of Escherichia coli in the acylation of lipid A: HtrB catalyzed incorporation of laurate. The Journal of Biological Chemistry, 271(20), 12095-12102. https://doi.org/10.1074/jbc.271.20.12095
Clementz, T., Zhou, Z., & Raetz, C. R. (1997). Function of the Escherichia coli msbB gene, a multicopy suppressor of htrB knockouts, in the acylation of lipid A. Acylation by MsbB follows laurate incorporation by HtrB. The Journal of Biological Chemistry, 272(16), 10353-10360. https://doi.org/10.1074/jbc.272.16.10353
Croll, T. I. (2018). ISOLDE: A physically realistic environment for model building into low-resolution electron-density maps. Acta Crystallographica. Section D, Structural Biology, 74(Pt 6), 519-530. https://doi.org/10.1107/S2059798318002425
Doerrler, W. T., & Raetz, C. R. H. (2002). ATPase activity of the MsbA lipid flippase of Escherichia coli. The Journal of Biological Chemistry, 277(39), 36697-36705. https://doi.org/10.1074/jbc.M205857200
Doerrler, W. T., Reedy, M. C., & Raetz, C. R. (2001). An Escherichia coli mutant defective in lipid export. The Journal of Biological Chemistry, 276(15), 11461-11464. https://doi.org/10.1074/jbc.C100091200
Emsley, P., & Cowtan, K. (2004). Coot: Model-building tools for molecular graphics. Acta Crystallographica. Section D, Biological Crystallography, 60(Pt 12 Pt 1), 2126-2132. https://doi.org/10.1107/S0907444904019158
Emsley, P., Lohkamp, B., Scott, W. G., & Cowtan, K. (2010). Features and development of Coot. Acta Crystallographica. Section D, Biological Crystallography, 66(Pt 4), 486-501. https://doi.org/10.1107/S0907444910007493
Garrett, T. A., Kadrmas, J. L., & Raetz, C. R. (1997). Identification of the gene encoding the Escherichia coli lipid A 4′-kinase. Facile phosphorylation of endotoxin analogs with recombinant LpxK. The Journal of Biological Chemistry, 272(35), 21855-21864. https://doi.org/10.1074/jbc.272.35.21855
Garrett, T. A., Que, N. L., & Raetz, C. R. (1998). Accumulation of a lipid A precursor lacking the 4′-phosphate following inactivation of the Escherichia coli lpxK gene. The Journal of Biological Chemistry, 273(20), 12457-12465. https://doi.org/10.1074/jbc.273.20.12457
Goddard, T. D., Huang, C. C., Meng, E. C., Pettersen, E. F., Couch, G. S., Morris, J. H., & Ferrin, T. E. (2018). UCSF ChimeraX: Meeting modern challenges in visualization and analysis. Protein Science: A Publication of the Protein Society, 27(1), 14-25. https://doi.org/10.1002/pro.3235
Goto, Y., Katoh, T., & Suga, H. (2011). Flexizymes for genetic code reprogramming. Nature Protocols, 6(6), 779-790. https://doi.org/10.1038/nprot.2011.331
Grant, T., Rohou, A., & Grigorieff, N. (2018). CisTEM, user-friendly software for single-particle image processing. ELife, 7, e35383. https://doi.org/10.7554/eLife.35383
Ho, H., Miu, A., Alexander, M. K., Garcia, N. K., Oh, A., Zilberleyb, I., Reichelt, M., Austin, C. D., Tam, C., Shriver, S., Hu, H., Labadie, S. S., Liang, J., Wang, L., Wang, J., Lu, Y., Purkey, H. E., Quinn, J., Franke, Y., . . . Koth, C. M. (2018). Structural basis for dual-mode inhibition of the ABC transporter MsbA. Nature, 557(7704), 196-201. https://doi.org/10.1038/s41586-018-0083-5
Hollenstein, K., Dawson, R. J. P., & Locher, K. P. (2007). Structure and mechanism of ABC transporter proteins. Current Opinion in Structural Biology, 17(4), 412-418. https://doi.org/10.1016/j.sbi.2007.07.003
Karow, M., & Georgopoulos, C. (1993). The essential Escherichia coli msbA gene, a multicopy suppressor of null mutations in the htrB gene, is related to the universally conserved family of ATP-dependent translocators. Molecular Microbiology, 7(1), 69-79. https://doi.org/10.1111/j.1365-2958.1993.tb01098.x
Kawakami, T., Ishizawa, T., Fujino, T., Reid, P. C., Suga, H., & Murakami, H. (2013). In vitro selection of multiple libraries created by genetic code reprogramming to discover macrocyclic peptides that antagonize VEGFR2 activity in living cells. ACS Chemical Biology, 8(6), 1205-1214. https://doi.org/10.1021/cb300697h
Laxminarayan, R., Duse, A., Wattal, C., Zaidi, A. K. M., Wertheim, H. F. L., Sumpradit, N., Vlieghe, E., Hara, G. L., Gould, I. M., Goossens, H., Greko, C., So, A. D., Bigdeli, M., Tomson, G., Woodhouse, W., Ombaka, E., Peralta, A. Q., Qamar, F. N., Mir, F., . . . Cars, O. (2013). Antibiotic resistance-the need for global solutions. The Lancet. Infectious Diseases, 13(12), 1057-1098. https://doi.org/10.1016/S1473-3099(13)70318-9
Mi, W., Li, Y., Yoon, S. H., Ernst, R. K., Walz, T., & Liao, M. (2017). Structural basis of MsbA-mediated lipopolysaccharide transport. Nature, 549(7671), 233-237. https://doi.org/10.1038/nature23649
Nikaido, H. (2003). Molecular basis of bacterial outer membrane permeability revisited. Microbiology and Molecular Biology Reviews: MMBR, 67(4), 593-656. https://doi.org/10.1128/MMBR.67.4.593-656.2003
Okuda, S., Freinkman, E., & Kahne, D. (2012). Cytoplasmic ATP hydrolysis powers transport of lipopolysaccharide across the periplasm in E. coli. Science (New York, N.Y.), 338(6111), 1214-1217. https://doi.org/10.1126/science.1228984
Onishi, H. R., Pelak, B. A., Gerckens, L. S., Silver, L. L., Kahan, F. M., Chen, M. H., Patchett, A. A., Galloway, S. M., Hyland, S. A., Anderson, M. S., & Raetz, C. R. (1996). Antibacterial agents that inhibit lipid A biosynthesis. Science (New York, N.Y.), 274(5289), 980-982. https://doi.org/10.1126/science.274.5289.980
Padayatti, P. S., Lee, S. C., Stanfield, R. L., Wen, P.-C., Tajkhorshid, E., Wilson, I. A., & Zhang, Q. (2019). Structural Insights into the Lipid A Transport Pathway in MsbA. Structure (London, England: 1993), 27(7), 1114-1123.e3. https://doi.org/10.1016/j.str.2019.04.007
Papadopoulos, J. S., & Agarwala, R. (2007). COBALT: Constraint-based alignment tool for multiple protein sequences. Bioinformatics (Oxford, England), 23(9), 1073-1079. https://doi.org/10.1093/bioinformatics/btm076
Pei, J., & Grishin, N. V. (2001). AL2CO: Calculation of positional conservation in a protein sequence alignment. Bioinformatics (Oxford, England), 17(8), 700-712. https://doi.org/10.1093/bioinformatics/17.8.700
Pettersen, E. F., Goddard, T. D., Huang, C. C., Meng, E. C., Couch, G. S., Croll, T. I., Morris, J. H., & Ferrin, T. E. (2021). UCSF ChimeraX: Structure visualization for researchers, educators, and developers. Protein Science: A Publication of the Protein Society, 30(1), 70-82. https://doi.org/10.1002/pro.3943
Polissi, A., & Georgopoulos, C. (1996). Mutational analysis and properties of the msbA gene of Escherichia coli, coding for an essential ABC family transporter. Molecular Microbiology, 20(6), 1221-1233. https://doi.org/10.1111/j.1365-2958.1996.tb02642.x
Raetz, C. R. H., & Whitfield, C. (2002). Lipopolysaccharide endotoxins. Annual Review of Biochemistry, 71, 635-700. https://doi.org/10.1146/annurev.biochem.71.110601.135414
Richter, M. F., Drown, B. S., Riley, A. P., Garcia, A., Shirai, T., Svec, R. L., & Hergenrother, P. J. (2017). Predictive compound accumulation rules yield a broad-spectrum antibiotic. Nature, 545(7654), 299-304. https://doi.org/10.1038/nature22308
Ruiz, N., Gronenberg, L. S., Kahne, D., & Silhavy, T. J. (2008). Identification of two inner-membrane proteins required for the transport of lipopolysaccharide to the outer membrane of Escherichia coli. Proceedings of the National Academy of Sciences of the United States of America, 105(14), 5537-5542. https://doi.org/10.1073/pnas.0801196105
Sampson, B. A., Misra, R., & Benson, S. A. (1989). Identification and characterization of a new gene of Escherichia coli K-12 involved in outer membrane permeability. Genetics, 122(3), 491-501. https://doi.org/10.1093/genetics/122.3.491
Scheres, S. H. W. (2012). RELION: Implementation of a Bayesian approach to cryo-EM structure determination. Journal of Structural Biology, 180(3), 519-530. https://doi.org/10.1016/j.jsb.2012.09.006
Sievers, F., Wilm, A., Dineen, D., Gibson, T. J., Karplus, K., Li, W., Lopez, R., McWilliam, H., Remmert, M., Söding, J., Thompson, J. D., & Higgins, D. G. (2011). Fast, scalable generation of high-quality protein multiple sequence alignments using Clustal Omega. Molecular Systems Biology, 7, 539. https://doi.org/10.1038/msb.2011.75
Storek, K. M., Auerbach, M. R., Shi, H., Garcia, N. K., Sun, D., Nickerson, N. N., Vij, R., Lin, Z., Chiang, N., Schneider, K., Wecksler, A. T., Skippington, E., Nakamura, G., Seshasayee, D., Koerber, J. T., Payandeh, J., Smith, P. A., & Rutherford, S. T. (2018). Monoclonal antibody targeting the B-barrel assembly machine of Escherichia coli is bactericidal. Proceedings of the National Academy of Sciences, 115(14), 3692-3697. https://doi.org/10.1073/pnas.1800043115
Studier, F. W. (2005). Protein production by auto-induction in high density shaking cultures. Protein Expression and Purification, 41(1), 207-234. https://doi.org/10.1016/j.pep.2005.01.016
Venter, J. C., Remington, K., Heidelberg, J. F., Halpern, A. L., Rusch, D., Eisen, J. A., Wu, D., Paulsen, I., Nelson, K. E., Nelson, W., Fouts, D. E., Levy, S., Knap, A. H., Lomas, M. W., Nealson, K., White, O., Peterson, J., Hoffman, J., Parsons, R., . . . Smith, H. O. (2004). Environmental genome shotgun sequencing of the Sargasso Sea. Science (New York, N.Y.), 304(5667), 66-74. https://doi.org/10.1126/science. 1093857
Vonrhein, C., Flensburg, C., Keller, P., Sharff, A., Smart, O., Paciorek, W., Womack, T., & Bricogne, G. (2011). Data processing and analysis with the autoPROC toolbox. Acta Crystallographica. Section D, Biological Crystallography, 67(Pt 4), 293-302. https://doi.org/10.1107/S0907444911007773
Wang, X., Karbarz, M. J., McGrath, S. C., Cotter, R. J., & Raetz, C. R. H. (2004). MsbA transporter-dependent lipid A 1-dephosphorylation on the periplasmic surface of the inner membrane: Topography of Francisella novicida LpxE expressed in Escherichia coli. The Journal of Biological Chemistry, 279(47), 49470-49478. https://doi.org/10.1074/jbc.M409078200
Wang, X., McGrath, S. C., Cotter, R. J., & Raetz, C. R. H. (2006). Expression cloning and periplasmic orientation of the Francisella novicida lipid A 4′-phosphatase LpxF. The Journal of Biological Chemistry, 281(14), 9321-9330. https://doi.org/10.1074/jbc.M600435200
Ward, A., Reyes, C. L., Yu, J., Roth, C. B., & Chang, G. (2007). Flexibility in the ABC transporter MsbA: Alternating access with a twist. Proceedings of the National Academy of Sciences of the United States of America, 104(48), 19005-19010. https://doi.org/10.1073/pnas.0709388104
Waterhouse, A., Bertoni, M., Bienert, S., Studer, G., Tauriello, G., Gumienny, R., Heer, F. T., de Beer, T. A. P., Rempfer, C., Bordoli, L., Lepore, R., & Schwede, T. (2018). SWISS-MODEL: Homology modelling of protein structures and complexes. Nucleic Acids Research, 46(W1), W296-W303. https://doi.org/10.1093/nar/gky427
Wu, T., McCandlish, A. C., Gronenberg, L. S., Chng, S.-S., Silhavy, T. J., & Kahne, D. (2006). Identification of a protein complex that assembles lipopolysaccharide in the outer membrane of Escherichia coli. Proceedings of the National Academy of Sciences of the United States of America, 103(31), 11754-11759. https://doi.org/10.1073/pnas.0604744103
Zhang, G., Baidin, V., Pahil, K. S., Moison, E., Tomasek, D., Ramadoss, N. S., Chatterjee, A. K., McNamara, C. W., Young, T. S., Schultz, P. G., Meredith, T. C., & Kahne, D. (2018). Cell-based screen for discovering lipopolysaccharide biogenesis inhibitors. Proceedings of the National Academy of Sciences of the United States of America, 115(26), 6834-6839. https://doi.org/10.1073/pnas.1804670115
Zhou, Z., White, K. A., Polissi, A., Georgopoulos, C., & Raetz, C. R. (1998). Function of Escherichia coli MsbA, an essential ABC family transporter, in lipid A and phospholipid biosynthesis. The Journal of Biological Chemistry, 273(20), 12466-12475. https://doi.org/10.1074/jbc.273.20.12466
Zoonens, M., & Popot, J.-L. (2014). Amphipols for each season. The Journal of Membrane Biology, 247(9-10), 759-796. https://doi.org/10.1007/s00232-014-9666-8

### SEQUENCES

The following table describes certain sequences referenced herein. Dashes are added between residues or functional groups of certain peptides listed below.

TABLE 7
Sequence Table.

| Seq Id. | Identity and/or | |
|---|---|---|
| No. | Description | Sequence |
| 1 | G1118 | CLAc-F-W-W-L-W-D-D-V-S-W-W-MeF-V-C-NH2 |
| 2 | G1118 | CLAc-F-W-W-L-W-D-D-V-S-W-W-MeF-V-C-G-K-K-NH2 |
| 3 | G1365 | CLAc-F-V-Y-Bph-MeF-R-V-C-NH2 |
| 4 | E. coli MsbA | MHNDKDLSTW QTFRRLWPTI APFKAGLIVA |
| | (Uniprot P60752; | GVALILNAAS DTEMLSLLKP LLDDGEGKTD |
| | NCBI | RSVLVWMPLV VIGLMILRGI TSYVSSYCIS |
| | NP_415434.1) | WVSGKVVMTM RRRLFGHMMG MPVSFEDKQS |
| | | TGTLLSRITY DSEQVASSSS GALITVVREG |
| | | ASIIGLFIMM FYYSWQLSII LIVLAPIVSI |
| | | AIRVVSKRER NISKNMQNTM GQVTTSAEQM |
| | | LKGHKEVLIF GGQEVETKRF DKVSNRMRLQ |
| | | GMKMVSASSI SDPIIQLIAS LALAFVLYAA |
| | | SFPSVMDSLT AGTITVVESS MIALMRPLKS |
| | | LTNVNAQFQR GMAACQTLFT ILDSEQEKDE |
| | | GKRVIERATG DVEFRNVTFT YPGRDVPALR |
| | | NINLKIPAGK TVALVGRSGS GKSTIASLIT |
| | | RFYDIDEGEI LMDGHDLREY TLASLRNQVA |
| | | LVSQNVHLEN DTVANNIAYA RTEQYSREQI |
| | | EEAARMAYAM DFINKMDNGL DTVIGENGVL |
| | | LSGGQRQRIA IARALLRDSP ILILDEATSA |
| | | LDTESERAIQ AALDELQKNR TSLVIAHRLS |
| | | TIEKADEIVV VEDGVIVERG THNDLLEHRG |
| | | VYAQLHKMQF GQ |
| 5 | Pseudomonas | MALLRHDETMSQDSPAQTSSLKIYFRLLGYVRPYIGMFALSI |
| | psychrotolerans | VGFLIFASTQPMLAGILKYFVDGLNNPNASMFPGWPIVGGLH |
| | MsbA (PpMsbA) | LVQAVPLMIILIAAWQGVGSFLGNYFLARVSLGLVHDLRVAL |
| | (Uniprot | FNHMLRLPNRYFDANNSGHLISRITFNVTMVTGAATEAIKVV |
| | A0A1G5PELO) | IREGMTVVFLFASLLWMNWKLTLVMVAILPLIALMVTSASRK |
| | | FRKQSSKIQSAMGDVTHITSEAIQGYRVVRSFGGERYEQQRF |
| | | LDSSTANTDKQLTMTKTGAVYTPMLQLVIYSAMAVLMFLVLW |
| | | LRGEASAGDLVAYITMAGLLPKPIRQLSEVSSTIQRGVAGAE |
| | | SIFEQLDEPAERDEGREERERVQGRLEVRNLTFRYPGTEKAV |
| | | LEGIDFVAEPGQMVALVGRSGSGKSTLANLIPRFYQHEEGEI |
| | | LLDGLEIQDYRLTNLRRHIALVTQHVTLENDSVINNIAYGDL |
| | | AGAPMEQVRAAAEAAYADEFIERLPQKYDTQVGENGVLLSGG |
| | | QRQRLAIARALLKNAPLLILDEATSALDTESERHIQAALDRV |
| | | MQGRTTLVIAHRLSTIEKADLILVMDNGRIVERGSHGELLAL |
| | | NGYYARLHAKQFEEVAPPAPTIEC |
| 6 | Candidatus | MPSLGTHLRLLGHLRPRLGLLGISLLGYLMFASSQPAMAAIL |
| | accumulibacter | KFFIDELGSPGSTRHALPWLGSVDLMHAVPLLIVAVIVWQGI |
| | MsbA (CaMsbA) | GSFLGSYFIARASLGLIDDLRSSLFDNLLRLPDAYFAHHHSG |
| | (Uniprot | HLVSRITYDVTMVTGAATDAVRVVIREALTIGFLFGYLLWLN |
| | A0A011NLL4) | WQLTLVLVAVLPLIGGIVASANRKYRQQSRNIQAAMGDLTQA |
| | | AAEMVQGHRVVRGFGGERYESARFQAASAENTAKQLRMVRTT |
| | | VSYSQIVQLITFSAMAVLLLLILLLRGDASAGDLVAYLTAAG |
| | | MLPKPVRQLSEMSATIQRGLAGAESIFAQLDEAPEPDRGTVE |
| | | RDRVRGFLEIRNLSFSYPDSSQRVLEGISFVAAPGQLIALVG |
| | | RSGSGKSSLASLIPRFYSPDEGQILIDGIDIEDYKLRNLRLH |
| | | IALVTQQVVLENDTITRNIAYGDLVVASPAVIREAAAAAHAC |
| | | EFIDQLPQGFDTLVGENGVMLSGGQRQRLAIARALLKDAPIL |
| | | ILDEATSALDTESERRIHNALDRIKERRTTLVIAHRLSTIER |
| | | ADLIIVIDDGRIVERGSHAELLAVDGHYARLHANQRSEQREI |
| | | AT |
| 7 | Janthinobacterium | MQQSRLLFFRLAGQFRRYGAIVAATLLAVGVASATDVLLIRQ |
| | agaricidamnosum | LQNVVDAMRTTASTQVAPVSGVMGMLQGWVDRFLPAQAGQVD |
| | MsbA (JaMsbA) | LWVIPATILGLAVLRMVSSFAGDYGSVWLSSRVQADLREKME |
| | (Uniprot | ATIMRLPSRFFDTTTTSLTQSRVAFDASQVSQAGLNVLNVMV |
| | A0A3G2E7N4) | RDSVATVGYLILLESIDVKLALFCMASMPIVAIVVTLAGRRM |
| | | RHLSKSSQQAVGELTSVLDESIGGQRVVKIFGGQDYEQARFG |
| | | NVVKRNRQLAVKHAATSAMNSGFIMMLVGITLSSVIYFAMLR |
| | | AQSGAITPGAFVAFMGALMAMQSPIKNLTKINEPLQRGLAAA |
| | | ESVFGLIDTPLEPDPGTIAPAAVQGNLSLQNVHFRYNSDDPD |
| | | APTALSDITLDIRAGETVALVGGSGGGKTTLLGLLPRFYDVS |
| | | GGRILLDGVDVRDYALLALRRQFALVSQDVILENDTMAANIA |
| | | YGDPAPDMARIEASARAAYAHDFIMQLPQGYATQAGQNGSRL |
| | | SGGQRQRLAIARALYKDAPILLLDEATSALDTESERSVQAAL |
| | | EVLMRNRTTIVIAHRLSTIESADRIVVMQGGRLVESGSHAAL |
| | | LQQGGAYARLHASQLSPVKDGMG |
| 8 | Thiomicrospira | MYDRQSLKLYGQLLRYILPYKSAIALTLFSLVLIALLEPATA |
| | cyclica MsbA | YVLKDLVDVGLIEQDPNSFVILPFLLALVFIFKGVFEYISKV |
| | (TcMsbA) | VSQWISERATLQIRLEMFEKLQFMTLKSFQDTTSGHLMSKIT |
| | (Uniprot F6DCY0) | HNVSQTSQALVNAWVVLIRDTLMIIALLAYMLYVSWELTLLM |
| | | LVIAPVVAFLINKASQLMRKYSRKSQENMGAVTQQLEESIHA |
| | | HKDIKVYGAEQYEIERFNQVLAKQMQYNVKQVRVAALNVPLV |
| | | QVIAAIALSFVVYFAMKLAADGAFTPGELVSFILAMALTEDP |
| | | IRRLTSVNITLQKGLAAAESIFEFLAITNETNTGTQQPPIKG |
| | | ELQLNNLVFQYDRAEQPTLKGIQLTIPANQTTALVGASGSGK |
| | | STLVNLIARFYAPTSGDILLDNTPLADIELDYLRQHIAFVSQ |
| | | HVVLENDSIRANIAYGHDEFDEAAIIQAAKDAHAWEFIEKLP |
| | | DGLDTMIGDNGALLSGGQRQRLAIARAFLKNAPILILDEATS |
| | | ALDNQSEAMIQQAMAKLRQNRTVIIIAHRLSTIEQADQIAVL |
| | | DQGELVELGTHRDLLAQQGAYARLQQQGELGSG |
| 9 | Magnetospira | MSEPESSARTKDPNATSVLLGRLLRENVAPYKWWIFWALLFM |
| | strain-QH-2 MsbA | AVVAASTGLTAYLLDPVITYIFDEKRHDLVWPLVAAILATFV |
| | (MqMsbA) | VKGGANYMQTLLMSRVGLRIIADNQNRLFRHLARMDLTFFHA |
| | (Uniprot | NPTGTLVSRFTVDIAQMRVAVSNGLTALGKDLLTLIALVGVM |
| | W6KCN7) | FYEDWQLSLIALVAFPTAIYPIARLGKRMRKVTANNQQEMGL |
| | | LITTLEQSFQGIRVVKAYGMEDYETERVVTLTDRIYALAVKA |
| | | ASTRAFSSPIMETLGGLAVAAVIAYAGNRVIHEDMRSGALIA |
| | | FIAALLMAYEPMKRLANLNASLQEGLAGAQRMFQLLDNPSDL |
| | | VEAVDAKDLDVAGGSVSLEKVRFSYDGENNALDGLSLEVPAG |
| | | STVALVGPSGAGKSTVLNLIPRFYDLNSGRVVIDGQDIKDVT |
| | | FTSLRGAMALVSQEITLEDDTVRANIAYGREGASEEDIQLAA |
| | | SHAAAHDFILKLPQGYDTMVGEQGVKLSGGQRQRLAIARAML |
| | | KNAPILLLDEATSALDTESERQVQAALDELMKDRTTLVIAHR |
| | | LSTVVQADLIHVVDRGRVVESGTHDQLLAADGIYARLYALQF |
| | | DEREEAVVETDPEVVGDDTP |
| 10 | G1119 | CLAc-F-W-W-L-W-S-D-MeG-D-W-W-MeF-V-C-NH2 |
| 11 | G1122 | CLAc-F-R-Y-L-W-MeA-W-G-L-V-W-D-N-C-NH2 |
| 12 | E. coli MsbA- | See FIG. 10A |
| | chimera1 protein | |
| | sequence | |
| 13 | E. coli MsbA- | See FIG. 10A |
| | chimera2 protein | |
| | sequence | |
| 14 | E. coli MsbA- | See FIG. 10A |
| | chimera3 protein | |
| | sequence | |
| 15 | E. coli MsbA- | See FIG. 10A |
| | chimera4 protein | |
| | sequence | |
| 16 | E. coli MsbA- | See FIG. 10A |
| | chimera5 protein | |
| | sequence | |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of determining whether a test molecule binds to the periplasmic, extracellular, and/or luminal face of a parental ABC transporter, comprising:
a) providing a chimeric ABC transporter, in which one or more regions of the periplasmic, extracellular, and/or luminal face of the parental ABC transporter are substituted with one or more equivalent regions of the periplasmic, extracellular, and/or luminal face of a different ABC transporter; and
b) contacting the chimeric ABC transporter with a molecule that binds to the parental ABC transporter in an outward-facing conformation, wherein the test molecule is determined to bind to the periplasmic, extracellular, and/or luminal face of the parental ABC transporter if the test molecule does not bind to the chimeric ABC transporter.

2. A method of determining whether a test molecule binds to the periplasmic, extracellular, and/or luminal face of a parental ABC transporter, comprising:
a) trapping the parental ABC transporter in an outward-facing conformation;
b) selecting a test molecule that binds to the parental ABC transporter in the outward-facing conformation;
c) providing a chimeric ABC transporter, in which one or more regions of the periplasmic, extracellular, and/or luminal face of the parental ABC transporter are substituted with one or more corresponding regions of the periplasmic, extracellular, and/or luminal face of a different ABC transporter; and
d) contacting the chimeric ABC transporter with the test molecule of (b) that binds to the parental ABC transporter in an outward-facing conformation, wherein the test molecule is determined to bind to the periplasmic, extracellular, and/or luminal face of the parental ABC transporter if the test molecule does not bind to the chimeric ABC transporter.

3. The method of claim 1, further comprising trapping the chimeric ABC transporter in an outward-facing conformation prior to contacting the chimeric ABC transporter with the test molecule.

4. The method of claim 3, wherein the parental ABC transporter and/or the chimeric ABC transporter is trapped in an outward-facing conformation by treating the parental ABC transporter and/or the chimeric ABC transporter with Mg²⁺, ATP, and vanadate.

5.-9. (canceled)

10. The method of claim 1, wherein the parental ABC transporter is MsbA.

11. (canceled)

12. (canceled)

13. The method of claim 1, wherein the chimeric ABC transporter differs from the parental ABC transporter in that at least one, at least two, at least three, or all of the periplasmic, extracellular, or luminal facing loop(s) and up to 50%, up to 25%, or up to 10% of transmembrane segments on either side of each loop are replaced with equivalent regions of the different ABC transporter.

14.-18. (canceled)

19. The method of claim 1, wherein the parental ABC transporter is E. coli MsbA (EcMsbA) and the chimeric ABC transporter is EcMsbA in which one or more of EcMsbA residues Leu47-Pro68 in periplasmic loop 1 (L1), EcMsbA residues Met159-Leu171 in periplasmic loop 3 (L3), and EcMsbA residues Ala262-Ile292 in periplasmic loop 5 (L5) are replaced with equivalent regions of Pseudomonas psychrotolerans (PpMsbA), Candidatus accumulibacter (CaMsbA), Janthinobacterium agaricidamnosum (JaMsbA), Thiomicrospira cyclica (TcMsbA), or Magnetospira strain-OH-2 (MqMsbA).

20.-26. (canceled)

27. A molecule identified by the method of claim 1, wherein the molecule binds to the parental ABC transporter with a K_{D} of 20 μM or less, 10 μM or less, 20 nM or less, 500 nM or less, 1 nM or less, 1 to 20 μM, 10 to 20 μM, 1 nM to 20 μM, or 1 nM to 500 nM, optionally wherein the molecule is a peptide, a small molecule, an antibody, a binding fragment of a peptide, small molecule, or antibody, or a macrocycle, further optionally wherein if the molecule is a macrocycle, the macrocycle is a 6-14-mer, 6-10-mer, 6-8-mer, or 8-10-mer macrocycle and/or comprises at least one lipophilic side-chain and at least one positively charged side-chain.

28.-43. (canceled)

44. A macrocycle peptide G1118 (SEQ ID NO: 1 or 2), G1119 (SEQ ID NO: 10), G1122 (SEQ ID NO: 11), or G1365 (SEQ ID NO: 3)

45.-49. (canceled)

50. A method of making the chimeric ABC transporter of claim 1, comprising replacing at least one periplasmic, extracellular, or luminal facing loop and up to 50% of the transmembrane segments on either side of the loop of the parental ABC transporter of claim 1 with equivalent regions of the different ABC transporter of claim 1.

51.-64. (canceled)

65. The chimeric ABC transporter of claim 1 in which at least one periplasmic, extracellular, or luminal facing loop and up to 50% of the transmembrane segments on either side of the loop of the parental ABC transporter of claim 1 are replaced with equivalent regions of the different ABC transporter of claim 1.

66.-78. (canceled)

79. A chimeric ABC transporter comprising a parental ABC transporter E. coli MsbA (EcMsbA) in which one or more of EcMsbA residues Leu47-Pro68 in periplasmic loop 1 (L1), EcMsbA residues Met159-Leu171 in periplasmic loop 3 (L3), and EcMsbA residues Ala262-Ile292 in periplasmic loop 5 (L5) are replaced with equivalent regions of Pseudomonas psychrotolerans (PpMsbA), Candidatus accumulibacter (CaMsbA), Janthinobacterium agaricidamnosum (JaMsbA), Thiomicrospira cyclica (TcMsbA), or Magnetospira strain-OH-2 (MqMsbA).

80.-83. (canceled)

84. A molecular complex comprising the chimeric ABC transporter of claim 65 bound to a peptide, small molecule, antibody, binding fragment of a peptide, binding fragment of a small molecule, or binding fragment of an antibody.

85.-90. (canceled)

91. A kit comprising the chimeric ABC transporter of claim 65 and reagents for carrying out a method of determining whether a test molecule binds to the periplasmic, extracellular, and/or luminal face of a parental ABC transporter, optionally wherein the chimeric ABC transporter is attached to a matrix or beads, and optionally wherein the kit further comprises one or more of the following:
a. a parental ABC transporter and/or a different ABC transporter from which the chimeric ABC transporter is engineered;
b. a matrix or beads for attachment of ABC transporters, optionally streptavidin-coated beads, avidin-coated beads, or deglycosylated-avidin-coated beads, or magnetic beads;
c. one or more detergents for solubilizing an ABC transporter on a matrix or beads;
d. at least one wash buffer;
e. at least one elution buffer;
f. at least one positive or negative control molecule.

92. (canceled)

93. A method of treating a bacterial infection in an individual comprising administering to the individual an effective amount of a molecule of claim 27.

94. (canceled)

95. A peptide comprising the following sequence:
(SEQ ID NO: 17)
ClacF-X1-X2-L-X3-X4-D-X5-X6-X7-X8-MeF-V-C,
wherein:
i. X1 is W, V, or Y;
ii. X2 is W or Y;
iii. X3 is W or Y;
iv. X4 is S, D, V, or H;
V. X5 is N, wherein N is chosen from any natural amino acid other than C, or a non-natural amino acid chosen from Bph ((S)-3-([1,1′-biphenyl]-4-yl)-2-aminopropanoic acid), Dopa (L-3,4-dihydroxyphenylalanine), MeF (N-methyl-L-phenylalanine), and MeG (N-methyl-L-glycine);
vi. X6 is Y, K, A, S, D, R, or V;
vii. X7 is W, Y, or Bph; and
viii. X8 is W or Y;
optionally wherein the peptide further comprises a G residue following the C residue at the C-terminal end, and wherein ClacF is N-chloroacetyl L-phenylalanine, Bph is (S)-3-([1,1′-biphenyl]-4-yl)-2-aminopropanoic acid, Dopa is L-3,4-dihydroxyphenylalanine, MeF is N-methyl-L-phenylalanine, and MeG is N-methyl-L-glycine.

96.-109. (canceled)

110. A peptide comprising the following amino acid sequence:
(SEQ ID NO: 19)
ClacF-X1-Y-Bph-MeF-X2-V-C,
wherein:
i. X1 is V, S, Y, W, Dopa, L, V, A, R, K, or D; and
ii. X2 is R, V, Dopa, or Y;
wherein ClacF is N-chloroacetyl L-phenylalanine, Bph is (S)-3-([1,1′-biphenyl]-4-yl)-2-aminopropanoic acid, Dopa is L-3,4-dihydroxyphenylalanine, and MeF is N-methyl-L-phenylalanine.

111.-118. (canceled)


SEQUENCE LISTING (19 sequences)

SEQ ID NO: 1 - protein, 14 aa, synthetic construct
  REGION 1..14: note = Synthetic: G1118
  MOD_RES 1: note = ClAc-F (N-chloroacetyl L-phenylalanine)
  MOD_RES 13: note = MeF (N-methyl-L-phenylalanine)
  MOD_RES 14: note = AMIDATION
FWWLWDDVSWWFVC

SEQ ID NO: 2 - protein, 17 aa, synthetic construct
  REGION 1..17: note = Synthetic: G1118
  MOD_RES 1: note = ClAc-F (N-chloroacetyl L-phenylalanine)
  MOD_RES 12: note = MeF (N-methyl-L-phenylalanine)
  MOD_RES 17: note = AMIDATION
FWWLWDDVSWWFVCGKK

SEQ ID NO: 3 - protein, 8 aa, synthetic construct
  REGION 1..8: note = Synthetic: G1365
  MOD_RES 1: note = ClAc-F (N-chloroacetyl L-phenylalanine)
  MOD_RES 4: note = X is Bph((S)-3-([1,1 -biphenyl]-4-yl)-2-aminopropanoic acid)
  MOD_RES 5: note = MeF (N-methyl-L-phenylalanine)
  MOD_RES 8: note = AMIDATION
FVYXFRVC

SEQ ID NO: 4 - protein, 582 aa, Escherichia coli
MHNDKDLSTWQTFRRLWPTIAPFKAGLIVAGVALILNAASDTFMLSLLKPLLDDGFGKTDRSVLVWMPLVVIGLMILRGITSYVSSYCISWVSGKVVMTMRRRLFGHMMGMPVSFFDKQSTGTLLSRITYDSEQVASSSSGALITVVREGASIIGLFIMMFYYSWQLSIILIVLAPIVSIAIRVVSKRFRNISKNMQNTMGQVTTSAEQMLKGHKEVLIFGGQEVETKRFDKVSNRMRLQGMKMVSASSISDPIIQLIASLALAFVLYAASFPSVMDSLTAGTITVVFSSMIALMRPLKSLTNVNAQFQRGMAACQTLFTILDSEQEKDEGKRVIERATGDVEFRNVTFTYPGRDVPALRNINLKIPAGKTVALVGRSGSGKSTIASLITRFYDIDEGEILMDGHDLREYTLASLRNQVALVSQNVHLFNDTVANNIAYARTEQYSREQIEEAARMAYAMDFINKMDNGLDTVIGENGVLLSGGQRQRIAIARALLRDSPILILDEATSALDTESERAIQAALDELQKNRTSLVIAHRLSTIEKADEIVVVEDGVIVERGTHNDLLEHRGVYAQLHKMQFGQ

SEQ ID NO: 5 - protein, 612 aa, Pseudomonas psychrotolerans
MALLRHDETMSQDSPAQTSSLKIYFRLLGYVRPYIGMFALSIVGFLIFASTQPMLAGILKYFVDGLNNPNASMFPGWPIVGGLHLVQAVPLMIILIAAWQGVGSFLGNYFLARVSLGLVHDLRVALFNHMLRLPNRYFDANNSGHLISRITFNVTMVTGAATEAIKVVIREGMTVVFLFASLLWMNWKLTLVMVAILPLIALMVTSASRKFRKQSSKIQSAMGDVTHITSEAIQGYRVVRSFGGERYEQQRFLDSSTANTDKQLTMTKTGAVYTPMLQLVIYSAMAVLMFLVLWLRGEASAGDLVAYITMAGLLPKPIRQLSEVSSTIQRGVAGAESIFEQLDEPAERDEGREERERVQGRLEVRNLTFRYPGTEKAVLEGIDFVAEPGQMVALVGRSGSGKSTLANLIPRFYQHEEGEILLDGLEIQDYRLTNLRRHIALVTQHVTLFNDSVTNNIAYGDLAGAPMEQVRAAAEAAYADEFIERLPQKYDTQVGENGVLLSGGQRQRLAIARALLKNAPLLILDEATSALDTESERHIQAALDRVMQGRTTLVIAHRLSTIEKADLILVMDNGRIVERGSHGELLALNGYYARLHAKQFEEVAPPAPTIEC

SEQ ID NO: 6 - protein, 590 aa, Candidatus accumulibacter
MPSLGTHLRLLGHLRPRLGLLGISLLGYLMFASSQPAMAAILKFFIDELGSPGSTRHALPWLGSVDLMHAVPLLIVAVIVWQGIGSFLGSYFIARASLGLIDDLRSSLFDNLLRLPDAYFAHHHSGHLVSRITYDVTMVTGAATDAVRVVIREALTIGFLFGYLLWLNWQLTLVLVAVLPLIGGIVASANRKYRQQSRNIQAAMGDLTQAAAEMVQGHRVVRGFGGERYESARFQAASAENTAKQLRMVRTTVSYSQIVQLITFSAMAVLLLLILLLRGDASAGDLVAYLTAAGMLPKPVRQLSEMSATIQRGLAGAESIFAQLDEAPEPDRGTVERDRVRGFLEIRNLSFSYPDSSQRVLEGISFVAAPGQLIALVGRSGSGKSSLASLIPRFYSPDEGQILIDGIDIEDYKLRNLRLHIALVTQQVVLFNDTITRNIAYGDLVVASPAVIREAAAAAHACEFIDQLPQGFDTLVGENGVMLSGGQRQRLAIARALLKDAPILILDEATSALDTESERRIHNALDRTKERRTTLVIAHRLSTIERADLIIVIDDGRIVERGSHAELLAVDGHYARLHANQRSEQREIAT

SEQ ID NO: 7 - protein, 611 aa, Janthinobacterium agaricidamnosum
MQQSRLLFFRLAGQFRRYGAIVAATLLAVGVASATDVLLIRQLQNVVDAMRTTASTQVAPVSGVMGMLQGWVDRFLPAQAGQVDLWVIPATILGLAVLRMVSSFAGDYGSVWLSSRVQADLREKMFATIMRLPSRFFDTTTTSLTQSRVAFDASQVSQAGLNVLNVMVRDSVATVGYLILLFSIDVKLALFCMASMPIVAIVVTLAGRRMRHLSKSSQQAVGELTSVLDESIGGQRVVKIFGGQDYEQARFGNVVKRNRQLAVKHAATSAMNSGFIMMLVGITLSSVIYFAMLRAQSGAITPGAFVAFMGALMAMQSPIKNLTKINEPLQRGLAAAESVFGLIDTPLEPDPGTIAPAAVQGNLSLQNVHFRYNSDDPDAPTALSDITLDIRAGETVALVGGSGGGKTTLLGLLPRFYDVSGGRILLDGVDVRDYALLALRRQFALVSQDVILFNDTMAANIAYGDPAPDMARIEASARAAYAHDFIMQLPQGYATQAGQNGSRLSGGQRQRLAIARALYKDAPILLLDEATSALDTESERSVQAALEVLMRNRTTIVIAHRLSTIESADRIVVMQGGRLVESGSHAALLQQGGAYARLHASQLSPVKDGMG

SEQ ID NO: 8 - protein, 579 aa, Thiomicrospira cyclica
MYDRQSLKLYGQLLRYILPYKSAIALTLFSLVLIALLEPATAYVLKDLVDVGLIEQDPNSFVILPFLLALVFIFKGVFEYISKVVSQWISERATLQIRLEMFEKLQFMTLKSFQDTTSGHLMSKITHNVSQTSQALVNAWVVLIRDTLMIIALLAYMLYVSWELTLLMLVIAPVVAFLINKASQLMRKYSRKSQENMGAVTQQLEESIHAHKDIKVYGAEQYEIERFNQVLAKQMQYNVKQVRVAALNVPLVQVIAAIALSFVVYFAMKLAADGAFTPGELVSFILAMALTFDPIRRLTSVNITLQKGLAAAESIFEFLAITNETNTGTQQPPIKGELQLNNLVFQYDRAEQPTLKGIQLTIPANQTTALVGASGSGKSTLVNLIARFYAPTSGDILLDNTPLADIELDYLRQHIAFVSQHVVLFNDSIRANIAYGHDEFDEAAIIQAAKDAHAWEFIEKLPDGLDTMIGDNGALLSGGQRQRLAIARAFLKNAPILILDEATSALDNQSEAMIQQAMAKLRQNRTVIIIAHRLSTIEQADQIAVLDQGELVELGTHRDLLAQQGAYARLQQQGELGSG

SEQ ID NO: 9 - protein, 608 aa, Magnetospira sp.
MSEPESSARTKDPNATSVLLGRLLRENVAPYKWWIFWALLFMAVVAASTGLTAYLLDPVITYIFDEKRHDLVWPLVAAILATFVVKGGANYMQTLLMSRVGLRIIADNQNRLFRHLARMDLTFFHANPTGTLVSRFTVDIAQMRVAVSNGLTALGKDLLTLIALVGVMFYEDWQLSLIALVAFPTAIYPIARLGKRMRKVTANNQQEMGLLTTTLEQSFQGIRVVKAYGMEDYETERVVTLTDRIYALAVKAASTRAFSSPIMETLGGLAVAAVIAYAGNRVIHEDMRSGALIAFIAALLMAYEPMKRLANLNASLQEGLAGAQRMFQLLDNPSDLVEAVDAKDLDVAGGSVSLEKVRFSYDGENNALDGLSLEVPAGSTVALVGPSGAGKSTVLNLIPRFYDLNSGRVVIDGQDIKDVTFTSLRGAMALVSQEITLFDDTVRANIAYGRFGASEEDIQLAASHAAAHDFILKLPQGYDTMVGEQGVKLSGGQRQRLAIARAMLKNAPILLLDEATSALDTESERQVQAALDELMKDRTTLVIAHRLSTVVQADLIHVVDRGRVVESGTHDQLLAADGIYARLYALQFDEREEAVVETDPEVVGDDTP

SEQ ID NO: 10 - protein, 14 aa, synthetic construct
  REGION 1..14: note = Synthetic: G1119
  MOD_RES 1: note = ClAc-F (N-chloroacetyl L-phenylalanine)
  MOD_RES 8: note = MeG (N-methyl-L-glycine)
  MOD_RES 12: note = MeF (N-methyl-L-phenylalanine)
  MOD_RES 14: note = AMIDATION
FWWLWSDGDWWFVC

SEQ ID NO: 11 - protein, 14 aa, synthetic construct
  REGION 1..14: note = Synthetic: G1122
  MOD_RES 1: note = ClAc-F (N-chloroacetyl L-phenylalanine)
  MOD_RES 6: note = MeA (N-methyl-L-alanine)
  MOD_RES 14: note = AMIDATION
FRYLWAWGLVWDNC

SEQ ID NO: 12 - protein, 591 aa, synthetic construct
  REGION 1..591: note = Synthetic: E. coli MsbA-chimera1 protein sequence
MHNDKDLSTWQTFRRLWPTIAPFKAGLIVAGVALILNAASDTFMLSLLKYFVDGLNNPNASMFPGWPIVGGLHLVQAVPLVVIGLMILRGITSYVSSYCISWVSGKVVMTMRRRLFGHMMGMPVSFFDKQSTGTLLSRITYDSEQVASSSSGALITVVREGASIIGLFISLLWMNWKLTLVMIVLAPIVSIAIRVVSKRFRNISKNMQNTMGQVTTSAEQMLKGHKEVLIFGGQEVETKRFDKVSNRMRLQGMKMVSASSISDPIIQLIASLAMAVLMFLVLWLRGEASAGDLVAYITMAGALMRPLKSLTNVNAQFQRGMAACQTLFTILDSEQEKDEGKRVIERATGDVEFRNVTFTYPGRDVPALRNINLKIPAGKTVALVGRSGSGKSTIASLITRFYDIDEGEILMDGHDLREYTLASLRNQVALVSQNVHLFNDTVANNIAYARTEQYSREQIEEAARMAYAMDFINKMDNGLDTVIGENGVLLSGGQRQRIAIARALLRDSPILILDEATSALDTESERAIQAALDELQKNRTSLVIAHRLSTIEKADEIVVVEDGVIVERGTHNDLLEHRGVYAQLHKMQFGQ

SEQ ID NO: 13 - protein, 590 aa, synthetic construct
  REGION 1..590: note = Synthetic: E. coli MsbA-chimera2 protein sequence
MHNDKDLSTWQTFRRLWPTIAPFKAGLIVAGVALILNAASDTFMLSLLKFFIDELGSPGSTRHALPWLGSVDLMHAVPLVVIGLMILRGITSYVSSYCISWVSGKVVMTMRRRLFGHMMGMPVSFFDKQSTGTLLSRITYDSEQVASSSSGALITVVREGASIIGLFIYLLWLNWQLTLVLIVLAPIVSIAIRVVSKRFRNISKNMQNTMGQVTTSAEQMLKGHKEVLIFGGQEVETKRFDKVSNRMRLQGMKMVSASSISDPIIQLIASLAMAVLLLLILLLRGDASAGDLVAYLTAAGALMRPLKSLTNVNAQFQRGMAACQTLFTILDSEQEKDEGKRVIERATGDVEFRNVTFTYPGRDVPALRNINLKIPAGKTVALVGRSGSGKSTIASLITRFYDIDEGEILMDGHDLREYTLASLRNQVALVSQNVHLFNDTVANNIAYARTEQYSREQIEEAARMAYAMDFINKMDNGLDTVIGENGVLLSGGQRQRIAIARALLRDSPILILDEATSALDTESERAIQAALDELQKNRTSLVIAHRLSTIEKADEIVVVEDGVIVERGTHNDLLEHRGVYAQLHKMQFGQ

SEQ ID NO: 14 - protein, 608 aa, synthetic construct
  REGION 1..608: note = Synthetic: E. coli MsbA-chimera3 protein sequence
MHNDKDLSTWQTFRRLWPTIAPFKAGLIVAGVALILNAASDTFMLSLLQNVVDAMRTTASTQVAPVSGVMGMLQGWVDRFLPAQAGQVDLWVIPLVVIGLMILRGITSYVSSYCISWVSGKVVMTMRRRLFGHMMGMPVSFFDKQSTGTLLSRITYDSEQVASSSSGALITVVREGASIIGLFILLFSIDVKLALFCIVLAPIVSIAIRVVSKRFRNISKNMQNTMGQVTTSAEQMLKGHKEVLIFGGQEVETKRFDKVSNRMRLQGMKMVSASSISDPIIQLIASLTLSSVIYFAMLRAQSGAITPGAFVAFMGALMALMRPLKSLTNVNAQFQRGMAACQTLFTILDSEQEKDEGKRVIERATGDVEFRNVTFTYPGRDVPALRNINLKIPAGKTVALVGRSGSGKSTIASLITRFYDIDEGEILMDGHDLREYTLASLRNQVALVSQNVHLFNDTVANNIAYARTEQYSREQIEEAARMAYAMDFINKMDNGLDTVIGENGVLLSGGQRQRIAIARALLRDSPILILDEATSALDTESERAIQAALDELQKNRTSLVIAHRLSTIEKADEIVVVEDGVIVERGTHNDLLEHRGVYAQLHKMQFGQ

SEQ ID NO: 15 - protein, 582 aa, synthetic construct
  REGION 1..582: note = Synthetic: E. coli MsbA-chimera4 protein sequence
MHNDKDLSTWQTFRRLWPTIAPFKAGLIVAGVALILNAASDTFMLSLLKDLVDVGLIEQDPNSFVILPLVVIGLMILRGITSYVSSYCISWVSGKVVMTMRRRLFGHMMGMPVSFFDKQSTGTLLSRITYDSEQVASSSSGALITVVREGASIIGLFIYMLYVSWELTLLMIVLAPIVSIAIRVVSKRFRNISKNMQNTMGQVTTSAEQMLKGHKEVLIFGGQEVETKRFDKVSNRMRLQGMKMVSASSISDPIIQLIASLALSFVVYFAMKLAADGAFTPGELVSFILAMAALMRPLKSLTNVNAQFQRGMAACQTLFTILDSEQEKDEGKRVIERATGDVEFRNVTFTYPGRDVPALRNINLKIPAGKTVALVGRSGSGKSTIASLITRFYDIDEGEILMDGHDLREYTLASLRNQVALVSQNVHLFNDTVANNIAYARTEQYSREQIEEAARMAYAMDFINKMDNGLDTVIGENGVLLSGGQRQRIAIARALLRDSPILILDEATSALDTESERAIQAALDELQKNRTSLVIAHRLSTIEKADEIVVVEDGVIVERGTHNDLLEHRGVYAQLHKMQFGQ

SEQ ID NO: 16 - protein, 582 aa, synthetic construct
  REGION 1..582: note = Synthetic: E. coli MsbA-chimera5 protein sequence
MHNDKDLSTWQTFRRLWPTIAPFKAGLIVAGVALILNAASDTFMLSLLDPVITYIFDEKRHDLVWPLVLVVIGLMILRGITSYVSSYCISWVSGKVVMTMRRRLFGHMMGMPVSFFDKQSTGTLLSRITYDSEQVASSSSGALITVVREGASIIGLFIVMFYEDWQLSLIAIVLAPIVSIAIRVVSKRFRNISKNMQNTMGQVTTSAEQMLKGHKEVLIFGGQEVETKRFDKVSNRMRLQGMKMVSASSISDPIIQLIASLAVAAVIAYAGNRVIHEDMRSGALIAFIAALLALMRPLKSLTNVNAQFQRGMAACQTLFTILDSEQEKDEGKRVIERATGDVEFRNVTFTYPGRDVPALRNINLKIPAGKTVALVGRSGSGKSTIASLITRFYDIDEGEILMDGHDLREYTLASLRNQVALVSQNVHLFNDTVANNIAYARTEQYSREQIEEAARMAYAMDFINKMDNGLDTVIGENGVLLSGGQRQRIAIARALLRDSPILILDEATSALDTESERAIQAALDELQKNRTSLVIAHRLSTIEKADEIVVVEDGVIVERGTHNDLLEHRGVYAQLHKMQFGQ

SEQ ID NO: 17 - protein, 14 aa, synthetic construct
  REGION 1..14: note = Synthetic: 14-mer macrocyclic peptide
  REGION 1..14: note = misc_feature - See specification as filed for detailed description of substitutions and preferred embodiments
  MOD_RES 1: note = ClAcF (N-chloroacetyl L-phenylalanine)
  VARIANT 2: note = X is W, V, or Y
  VARIANT 3: note = X is W or Y
  VARIANT 5: note = X is W or Y
  VARIANT 6: note = X is S, D, V, or H
  VARIANT 8: note = X any natural amino acid other than C, or a non-natural amino acid chosen from Bph, Dopa, MeF, and MeG
  VARIANT 9: note = X is Y, K, A, S, D, R, or V
  VARIANT 10: note = X is W, Y, or Bph
  VARIANT 11: note = X is W or Y
  MOD_RES 12: note = MeF (N-methyl-L-phenylalanine)
FXXLXXDXXXXFVC

SEQ ID NO: 18 - protein, 15 aa, synthetic construct
  REGION 1..15: note = Synthetic: recognition motif for E. coli BirA biotin ligase
GLNDIFEAQKIEWHE

SEQ ID NO: 19 - protein, 8 aa, synthetic construct
  REGION 1..8: note = Synthetic
  REGION 1..8: note = misc_feature - See specification as filed for detailed description of substitutions and preferred embodiments
  MOD_RES 1: note = ClacF (N-chloroacetyl L-phenylalanine)
  VARIANT 2: note = X is V, S, Y, W, Dopa, L, V, A, R, K, or D
  MOD_RES 4: note = X is Bph((S)-3-([1,1 -biphenyl]-4-yl)-2-aminopropanoic acid)
  MOD_RES 5: note = MeF (N-methyl-L-phenylalanine)
  VARIANT 6: note = X is R, V, Dopa, or Y
FXYXFXVC